(19) United States
(12) Patent Application Publication
(10) Pub. No.: US 2017/0183650 A1
(43) Pub. Date: Jun. 29, 2017

(54) PHARMACEUTICAL COMPOSITIONS COMPRISING RNA AND USE FOR TREATING CANCER

(71) Applicant: Andes Biotechnologies S.A., Santiago (CL)

(72) Inventors: Luis O. BURZIO ERIZ, Santiago (CL); Veronica A. BURZIO MENENDEZ, Santiago (CL)

(21) Appl. No.: 15/125,947

(22) PCT Filed: Mar. 13, 2015
(86) PCT No.: PCT/IB15/01233

§ 371 Date: Sep. 13, 2016

Related U.S. Application Data
(60) Provisional application No. 61/953,672, filed on Mar. 14, 2014.

(51) Int. Cl. C12N 15/113

(52) U.S. Cl. CPC C12N 15/113; C12N 2310/113; C12N 2310/315; C12N 2330/00

(57) ABSTRACT

Methods are provided for making an RNA molecule derived from non-coding chimeric mitochondrial RNAs (ncmtRNAs), in particular antisense non-coding chimeric mitochondrial RNAs (ASncmtRNAs), compositions containing the isolated RNA molecule, methods of causing apoptosis in a cancer cell by contacting the cell with the RNA molecule, and methods of treating cancers by administering the RNA molecule to a subject in need thereof.

### CROSS-REFERENCE TO RELATED PATENT APPLICATIONS

This application claims the priority benefit of U.S. Provisional Application Ser. No. 61/953,672, filed Mar. 14, 2014, which is incorporated herein by reference in its entirety.

### FIELD OF THE INVENTION

This invention provides an isolated RNA molecule, methods of making thereof, and use thereof for treating various cancers. Such an RNA molecule and equivalents are prepared by the processing of non-coding chimeric mitochondrial RNA to provide intermediate RNA molecules, and fully processed mitochondrial microRNA.

### BACKGROUND OF THE INVENTION

There are a number of fundamental principles involved in human cancer progression. These include sustained proliferation, immortality, refractoriness to growth suppressors, resistance to cell death, angiogenic capability and induction of invasion and metastasis, promotion by genome instability, mutation and inflammation. Such hallmarks of cancer are the result of various cause and effects, such that various pathways and mechanisms result in a cancerous cell. Many cancers are readily treatable, while others are resistant to many types of treatment. Finding a treatment that is adaptable to many cancers, yet has few side effects with respect to normal cells is the consummate goal of cancer therapy.

Differential expression in human cells of a unique family of non-coding chimeric mitochondrial long ncRNAs (ncmtRNAs) has been observed. These ncmtRNAs contain a stem-loop structure (Villegas et al., Nucleic Acids Res., 35:7336-7347, 2007; Burzio et al., Proc. Natl. Acad. Sci. U.S.A., 106:9430-9434, 2009). These transcripts also exit the mitochondria to the cytosol and nucleus (Landerer et al., Cell Oncol., 34:297-305, 2011). One such transcript, the sense ncmtRNA (SncmtRNA) is expressed in normal proliferating cells and in tumor cells, but not in resting cells (Villegas et al., supra; Burzio et al., supra; Villota et al., J. Biol. Chem., 287:21303-21315, 2012). Normal human proliferating cells also express antisense non-coding chimeric mitochondrial RNAs (ASncmtRNA, which are down-regulated in multiple tumor cell lines and tumor cells present in seventeen types of cancer biopsies from different patients (Burzio et al., supra). This down-regulation of the ASncmtRNAs represents another hallmark of cancer (Hannahan et al., Cell, 144:646-674, 2011). Antisense oligonucleotides directed to the ASncmtRNAs have been shown to decrease proliferation of HeLa cells and several other tumor cell lines, without affecting normal cells.

Such a vulnerability of cancer cells that can be used against all types of cancers may bring us one step closer to finding a universal cancer treatment. The impact of this effect of down-regulation of the ASncmtRNAs provides the possibility of a treatment option that will work on virtually any cancer. As such, there is a need to provide additional nucleic acid molecules capable of exploiting this vulnerability of human cancers.

### SUMMARY OF THE INVENTION

Provided herein, inter alia, are methods for making an RNA molecule derived from non-coding chimeric mitochondrial RNAs (ncmtRNAs), in particular antisense non-coding chimeric mitochondrial RNAs (ASncmtRNAs), to compositions containing the isolated RNA molecule, to methods of causing apoptosis in a cancer cell by contacting the cell with the RNA molecule, to methods of treating cancers or pre-cancers by administering the RNA molecule to a subject in need thereof, and to methods of eliminating cancer stem cells by administering the RNA molecule to a subject in need thereof.

Accordingly, in one aspect, provided herein is a method for preparing an isolated RNA molecule comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) optionally digesting the non-coding chimeric mitochondrial RNA molecule cleaved by RNase H with an exonuclease to provide a non-coding chimeric mitochondrial RNA molecule sequentially cleaved by RNase H and exonuclease; (c) optionally digesting the non-coding chimeric mitochondrial RNA molecule sequentially cleaved by RNase H and exonuclease with Dicer to provide a non-coding chimeric mitochondrial RNA molecule sequentially cleaved by RNase H, exonuclease and Dicer; and (d) isolating the non-coding chimeric mitochondrial RNA molecule cleaved by RNase H, and/or optionally isolating the non-coding chimeric mitochondrial RNA molecule sequentially cleaved by RNase H and exonuclease, and/or optionally isolating the non-coding chimeric mitochondrial RNA molecule sequentially cleaved by RNase H, exonuclease and Dicer, to provide the isolated RNA molecule.

In another aspect, provided herein is an isolated RNA molecule, wherein said isolated RNA molecule is prepared by a method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) optionally digesting the non-coding chimeric mitochondrial RNA molecule cleaved by RNase H with an exonuclease to provide a non-coding chimeric mitochondrial RNA molecule sequentially cleaved by RNase H and exonuclease; (c) optionally digesting the non-coding chimeric mitochondrial RNA molecule sequentially cleaved by RNase H and exonuclease with Dicer to provide a non-coding chimeric mitochondrial RNA molecule sequentially cleaved by RNase H, exonuclease and Dicer; and isolating the non-coding chimeric mitochondrial RNA molecule cleaved by RNase H, and/or optionally isolating the non-coding chimeric mitochondrial RNA molecule sequentially cleaved by RNase H and exonuclease, and/or optionally isolating the non-coding chimeric mitochondrial RNA molecule sequentially cleaved by RNase H, exonuclease and Dicer, to provide the isolated RNA molecule.

In another aspect, provided herein is an isolated RNA molecule, wherein said isolated RNA molecule comprises a sequence analogous to an RNA molecule selected from the group consisting of a non-coding chimeric mitochondrial RNA molecule cleaved by RNase H; a non-coding chimeric mitochondrial RNA molecule sequentially cleaved by RNase H and exonuclease; and a non-coding chimeric mitochondrial RNA molecule sequentially cleaved by RNase H, exonuclease and Dicer; wherein the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer are prepared by a method comprising (a) annealing one or more oligonucleotides with a non-coding chimeric mitochondrial RNA molecule and digesting with an RNase H to provide the non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide the non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the non-coding chimeric mitochondrial RNA molecule sequentially cleaved by RNase H, exonuclease and Dicer.

In another aspect, provided herein is a synthetic microRNA molecule or synthetic DNA analog thereof, wherein the synthetic microRNA molecule comprises a sequence analogous to an RNA molecule resulting from a non-coding chimeric mitochondrial RNA molecule sequentially cleaved by RNase H, exonuclease and Dicer; wherein the non-coding chimeric mitochondrial RNA molecule sequentially cleaved by RNase H, exonuclease and Dicer is prepared by a method comprising (a) annealing one or more oligonucleotides with a non-coding chimeric mitochondrial RNA molecule and digesting with an RNase H to provide the non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide the non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the non-coding chimeric mitochondrial RNA molecule sequentially cleaved by RNase H, exonuclease and Dicer.

In another aspect, provided herein is a pharmaceutical composition comprising one or more isolated RNA molecules, wherein said isolated RNA molecule is prepared by a method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) optionally digesting the non-coding chimeric mitochondrial RNA molecule cleaved by RNase H with an exonuclease to provide a non-coding chimeric mitochondrial RNA molecule sequentially cleaved by RNase H and exonuclease; (c) optionally digesting the non-coding chimeric mitochondrial RNA molecule sequentially cleaved by RNase H and 5′ exonuclease with Dicer to provide a non-coding chimeric mitochondrial RNA molecule sequentially cleaved by RNase H, exonuclease and Dicer; and isolating the non-coding chimeric mitochondrial RNA molecule cleaved by RNase H, and/or optionally isolating the non-coding chimeric mitochondrial RNA molecule sequentially cleaved by RNase H and exonuclease, and/or optionally isolating the non-coding chimeric mitochondrial RNA molecule sequentially cleaved by RNase H, exonuclease and Dicer, to provide the isolated RNA molecule.

In another aspect, provided herein is a pharmaceutical composition comprising one or more isolated RNA molecules, wherein said one or more RNA molecules comprises a sequence analogous to an RNA molecule selected from the group consisting of a non-coding chimeric mitochondrial RNA molecule cleaved by RNase H; a non-coding chimeric mitochondrial RNA molecule sequentially cleaved by RNase H and exonuclease; and a non-coding chimeric mitochondrial RNA molecule sequentially cleaved by RNase H, exonuclease and Dicer; wherein the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer are prepared by a method comprising (a) annealing one or more oligonucleotides with a non-coding chimeric mitochondrial RNA molecule and digesting with an RNase H to provide the non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide the non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the non-coding chimeric mitochondrial RNA molecule sequentially cleaved by RNase H, exonuclease and Dicer.

In another aspect, provided herein is a pharmaceutical composition comprising one or more synthetic microRNA molecules or synthetic DNA analog thereof, wherein said one or more RNA molecules comprises a sequence analogous to an RNA molecule resulting from a non-coding chimeric mitochondrial RNA molecule sequentially cleaved by RNase H, exonuclease and Dicer; wherein the non-coding chimeric mitochondrial RNA molecule sequentially cleaved by RNase H, exonuclease and Dicer is prepared by a method comprising (a) annealing one or more oligonucleotides with a non-coding chimeric mitochondrial RNA molecule and digesting with an RNase H to provide the non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide the non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the non-coding chimeric mitochondrial RNA molecule sequentially cleaved by RNase H, exonuclease and Dicer.

In another aspect, provided herein is a method of causing apoptosis in a tumor cell comprising contacting the tumor cell with one or more isolated RNA molecules or pharmaceutical composition comprising the one or more isolated RNA molecules as described herein.

In another aspect, provided herein is a method of treating a cancer or pre-cancer, or to eliminate cancer stem cells in a subject comprising administering to the subject in need thereof a therapeutically effective amount of the isolated RNA molecule or pharmaceutical composition comprising the isolated RNA molecule as described herein.

In another aspect, provided herein are kits for use in the treatment of cancer, the kit comprising the isolated RNA molecule or pharmaceutical composition comprising the isolated RNA molecule as described herein. In some embodiments, the kit comprises instructions for use of the isolated RNA molecule or pharmaceutical composition comprising the isolated RNA molecule in the treatment of cancer.

In another aspect, provided herein is an article of manufacture, such as a container comprising a unit dosage form of an isolated RNA molecule as described herein, or a pharmaceutical composition comprising the isolated RNA molecule, and a label containing instructions for use of the isolated RNA molecule in the treatment of cancer.

In another aspect, provided herein is an isolated RNA molecule as described herein, or a pharmaceutical composition comprising the isolated RNA molecule, for use in the treatment of a cancer.

In another aspect, provided herein is an isolated RNA molecule as described herein, or a pharmaceutical composition comprising the isolated RNA molecule, for use in the manufacture of a medicament for the treatment of a cancer.

### BRIEF DESCRIPTION OF THE DRAWINGS

FIG. 1 provides the DNA sequence of SEQ ID NO: 1 (FIG. 1A) and the RNA sequence (SEQ ID NO:161, FIG. 1B) corresponding to SEQ ID NO:1.

FIG. 2 provides the DNA sequence of SEQ ID NO:2 (FIG. 2A) and the RNA sequence (SEQ ID NO:162, FIG. 2B) corresponding to SEQ ID NO:2.

FIG. 3 provides the DNA sequence of SEQ ID NO:3 (FIG. 3A) and the RNA sequence (SEQ ID NO:163, FIG. 3B) corresponding to SEQ ID NO:3.

FIG. 4 provides the DNA sequence of SEQ ID NO:4 (FIG. 4A) and the RNA sequence (SEQ ID NO:164, FIG. 4B) corresponding to SEQ ID NO:4.

FIG. 5 provides the DNA sequence of SEQ ID NO:5 (FIG. 5A) and the RNA sequence (SEQ ID NO:165, FIG. 5B) corresponding to SEQ ID NO:5.

FIG. 6 provides the DNA sequence of SEQ ID NO:6 (FIG. 6A) and the RNA sequence (SEQ ID NO:166, FIG. 6B) corresponding to SEQ ID NO:6.

FIG. 7 provides a schematic representation of the processing of a non-coding chimeric mitochondrial RNA molecule using an oligonucleotide complementary to the loop region (FIG. 7A) or the 3′ single stranded stem region (FIG. 7B).

FIG. 8 provides a schematic representation of exemplary oligonucleotides complementary to the loop region (1537S) or the 3′ single stranded stem region (126S, 552S, or 1107S).

FIG. 9 shows the knockdown of AS-1 or AS-2 RNA on polyacrylamide gel after transfection with ASO-1537S in three cell lines, cancer cells HeLa (FIG. 9A) and SK-MEL-2 (FIG. 9C) and normal HFK cells (FIG. 9B).

FIG. 10 shows the cell proliferation of HeLa cells without treatment, or transfected with ASO-C (control) or with ASO-1537S complementary to the loop region of the non-coding chimeric mitochondrial RNA.

FIG. 11 shows the relative amount of 5-ethynyl-2′-deoxyuridine (EdU) incorporated into HeLa cells that are untreated (NT), transfected with ASO-C (control) or with ASO-1537S complementary to the loop region of the non-coding chimeric mitochondrial RNA.

FIG. 12 shows the % Trypan blue positive HeLa cells that are untreated (NT), transfected with 50 nM, 75 nM or 100 nM ASO-C (control) or with 50 nM, 75 nM or 100 nM ASO-1537S complementary to the loop region of the non-coding chimeric mitochondrial RNA.

FIG. 13 shows the % Trypan blue positive HeLa cells that are untreated (NT), transfected with ASO-C (control) or with ASO-126S, ASO-552S, or ASO-1107S complementary to the 3′ stem region of the non-coding chimeric mitochondrial RNA.

FIG. 14 shows the % Trypan blue (Tb) negative cells in three normal cell lines, HUVEC (FIG. 14A), HREC (FIG. 14B) and HnEM (FIG. 14C), that are untreated (NT), transfected with ASO-C (control) or with ASO-1537S complementary to the loop region of the non-coding chimeric mitochondrial RNA.

FIG. 15 shows the cell analysis by Flow Cytometry of HeLa cells that are untreated (NT), transfected with ASO-C (control) or with ASO-1537S complementary to the loop region of the non-coding chimeric mitochondrial RNA, or treated with carbonyl cyanide m-chlorophenyl hydrazine as a positive control. Cells were loaded post treatment with fluorescent probe tetramethylrhodamine methyl ester to assess dissipation of the mitochondrial membrane potential ΔΨm.

FIG. 16 shows the % dissipation of ΔΨm (white portion of bar) for HeLa cells that are untreated (NT), transfected with ASO-C (control) or with ASO-1537S complementary to the loop region of the non-coding chimeric mitochondrial RNA, or treated with carbonyl cyanide m-chlorophenyl hydrazine as a positive control.

FIG. 17 shows the Flow Cytometry of DU145 (FIG. 17A), MDA-MB-231(FIG. 17B) or H 292 (FIG. 17C) cells that are untreated (NT), transfected with ASO-C (control) or with ASO-1537S complementary to the loop region of the non-coding chimeric mitochondrial RNA, or treated with carbonyl cyanide m-chlorophenyl hydrazine as a positive control. Cells were loaded post treatment with fluorescent probe tetramethylrhodamine methyl ester to assess dissipation of ΔΨm.

FIG. 18 shows the western blot analysis for cytochrome c and β-actin in HeLa cells that are untreated (NT), transfected with ASO-C (control) or with ASO-1537S complementary to the loop region of the non-coding chimeric mitochondrial RNA, or treated with staurosporine (STP) as a positive control.

FIG. 19 shows the % caspase positive cells in HeLa cells that are untreated (NT), transfected with ASO-C (control) or with ASO-1537S complementary to the loop region of the non-coding chimeric mitochondrial RNA, or treated with staurosporine (STP) as a positive control.

FIG. 20 shows the % propidium iodide positive cells in HeLa cells that are untreated (NT), transfected with ASO-C (control) or with ASO-1537S complementary to the loop region of the non-coding chimeric mitochondrial RNA, or treated with staurosporine (STP) as a positive control, with or without pretreatment with caspase inhibitor z-VAD-fmk.

FIG. 21 shows the % Annexin V positive cells in HeLa cells that are untreated (NT), transfected with ASO-C (control) or with ASO-1537S complementary to the loop region of the non-coding chimeric mitochondrial RNA, or treated with staurosporine (STP) as a positive control.

FIG. 22 shows HeLa (22A) or HFK (22B) cells transfected with ASO-C (control) or with ASO-1537S complementary to the loop region of the non-coding chimeric mitochondrial RNA, or treated with Dnase I as a positive control, stained using a TUNEL assay or with 4′,6-diamidino-2-phenylindole (DAPI).

FIG. 23 shows the % TUNEL positive cells in cancer cell lines of MCF7, MDA-MB-231, HepG2, SiHa, DU145, PC3, OVCAR-3, SK-MEL-2, Caco-2, A498, and U87 transfected with ASO-C (control) or with ASO-1537S complementary to the loop region of the non-coding chimeric mitochondrial RNA.

FIG. 24 shows the Flow Cytometry results to assess sub G1 fraction of cells for HeLa cells that are untreated (NT), transfected with ASO-C (control) or with ASO-1537S complementary to the loop region of the non-coding chimeric mitochondrial RNA, or treated with staurosporine (STP) as a positive control.

FIG. 25 shows the Flow Cytometry results of a triplicate analysis to assess sub G1 fraction of cells for HeLa cells that are untreated (NT), transfected with ASO-C (control) or with ASO-1537S complementary to the loop region of the non-coding chimeric mitochondrial RNA, or treated with staurosorine (STP) as a positive control.

FIG. 26 shows the sub G1 fraction of cells for HFK cells that are untreated (NT), transfected with ASO-C (control) or with ASO-1537S complementary to the loop region of the non-coding chimeric mitochondrial RNA, or treated with staurosporine (STP) as a positive control.

FIG. 27 shows the number of colonies over 50 μm on average of three wells for untreated (NT) and ASO-C treated cells, and the total number of such colonies for all three wells for ASO-1537S treated cells in four cancer cell lines, SK-MEL-2 (FIG. 27A), OVCAR-3 (FIG. 27B), HeLa (FIG. 27C) and SiHa (FIG. 27D).

FIG. 28 shows the western blot analysis for survivin and β-actin in SK-MEL-2 cells that are untreated (NT), transfected with ASO-C (control) or with ASO-1537S complementary to the loop region of the non-coding chimeric mitochondrial RNA for 24 hours (FIG. 28A), or at 3, 8, and 22 hours (FIG. 28B).

FIG. 29 shows the survivin expression normalized to β-actin in SK-MEL-2 cells that are untreated (NT), transfected with ASO-C (control) or with ASO-1537S complementary to the loop region of the non-coding chimeric mitochondrial RNA for 24 hours.

FIG. 30 shows the western blot analysis for survivin and β-actin in SK-MEL-2 cells that are transfected with ASO-C (control) or with ASO-1537S complementary to the loop region of the non-coding chimeric mitochondrial RNA for 24 hours with or without pretreatment with proteosome inhibitor MG132 (FIG. 30A) or caspase inhibitor z-VAD-fmk (FIG. 30B).

FIG. 31 shows the relative expression of survivin mRNA in SK-MEL-2 cells that are untreated (NT), transfected with ASO-C (control) or with ASO-1537S complementary to the loop region of the non-coding chimeric mitochondrial RNA for 24 hours.

FIG. 32 shows the western blot analysis for survivin and β-actin in three cancer cell lines, PC3 (FIG. 32A), OVCAR-3 (FIG. 32B) or H292 (FIG. 32C) that are untreated (NT) or transfected with ASO-C (control) or with ASO-1537S complementary to the loop region of the non-coding chimeric mitochondrial RNA for 24 hours.

FIG. 33 shows the western blot analysis for Bcl-2 and β-actin in two cancer cell lines, SK-MEL-2 (FIG. 33A) or PC3 (FIG. 33B) that are untreated (NT) or transfected with ASO-C (control) or with ASO-1537S complementary to the loop region of the non-coding chimeric mitochondrial RNA for 24 hours.

FIG. 34 shows the western blot analysis (FIG. 34A) for cyclin D1 and β-actin in SK-MEL-2 cells that are untreated (NT) or transfected with ASO-C (control) or with ASO-1537S complementary to the loop region of the non-coding chimeric mitochondrial RNA for 24 hours. FIG. 34B shows the cyclin D1 expression normalized to β-actin.

FIG. 35 shows the western blot analysis (FIG. 35A) for cyclin B1 and β-actin in SK-MEL-2 cells that are untreated (NT) or transfected with ASO-C (control) or with ASO-1537S complementary to the loop region of the non-coding chimeric mitochondrial RNA or ASO-226S complementary to the 3′ single stranded stem region of the non-coding chimeric mitochondrial RNA for 24 hours. FIG. 35B shows the cyclin B1 expression normalized to β-actin.

FIG. 36 shows the western blot analysis for N-cadherin, Caveolin, and β-actin in SK-MEL-2 cells that are untreated (NT) or transfected with ASO-C (control) or with ASO-1537S complementary to the loop region of the non-coding chimeric mitochondrial RNA for 24 hours.

FIG. 37 shows the western blot analysis for Bcl-2 (FIG. 37A), or chaperone FKBP38 (FIG. 37B) and β-actin in SK-MEL-2 cells that are untreated (NT) or transfected with ASO-C (control) or with ASO-1537S complementary to the loop region of the non-coding chimeric mitochondrial RNA or ASO-226S complementary to the 3′ single stranded stem region of the non-coding chimeric mitochondrial RNA for 24 hours.

FIG. 38 shows the western blot analysis for Bcl-2 (FIG. 38A), or chaperone FKBP38 (FIG. 38B) and β-actin in PC3 cells that are untreated (NT) or transfected with ASO-C (control) or with ASO-1537S complementary to the loop region of the non-coding chimeric mitochondrial RNA or ASO-226S complementary to the 3′ single stranded stem region of the non-coding chimeric mitochondrial RNA for 24 hours.

FIG. 39 shows the relative proliferation (FIG. 39A) and % Trypan blue (Tb) positive cells (FIG. 39B) in SK-MEL-2 cells that are untreated (NT) or transfected with ASO-C (control) or with ASO-1537S or with peptidic nucleic acid (PNA) analogs of ASO-C and ASO-1537S.

FIG. 40 shows the AS-1 and AS-2 levels by RT-PCR in SK-MEL-2 cells that are untreated (NT) or transfected with ASO-C (control) or with ASO-1537S or with peptidic nucleic acid (PNA) analogs of ASO-C and ASO-1537S.

FIG. 41 shows the western blot analysis for survivin and β-actin in SK-MEL-2 cells that are untreated (NT) or transfected with ASO-C (control) or with ASO-1537S or with peptidic nucleic acid (PNA) analogs of ASO-C and ASO-1537S.

FIG. 42 shows the gel analysis of RT-PCR amplification of AS-1 and AS-2 (FIG. 42A) in SK-MEL-2 cells with immunoprecipitation of the cytosolic fraction with anti-Dicer enzyme antibody. A positive control for immunoprecipitation is also included (42B).

FIG. 43 shows in silico generated Mito-miRs aligned with the 3′ UTR region of survivin mRNA (FIG. 43A), cyclin D1 mRNA (FIG. 43B), and cyclin B1 mRNA (FIG. 43C).

FIG. 44 shows a construct (44A) containing the 3′ UTR of survivin mRNA between the Firefly Luciferase and Renilla Luciferase coding regions (ORFs) to test in vivo the generation of microRNAs. FIG. 44B shows that inhibition of luminescence produced by ASO-1537S or Has-miR-218 (mRNA specific for 3″UTR of survivin) when transfected into SK-MEL-2 cells previously transfected with the construct containing the 3′ UTR of survivin.

### DETAILED DESCRIPTION OF THE INVENTION

Provided herein, inter alia, are methods for making an isolated RNA molecule derived from non-coding chimeric mitochondrial RNAs (ncmtRNAs), in particular antisense non-coding chimeric mitochondrial RNAs (ASncmtRNAs), compositions containing the isolated RNA molecule, methods of causing apoptosis in a cancer cell by contacting the cell with the isolated RNA molecule, and methods of treating cancers by administering the isolated RNA molecule to a subject in need thereof.

### I. General Techniques

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manua,l Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons, Inc., New York, 2000), Handbook of Experimental Immunology, 4th edition (D. M. Weir & C. C. Blackwell, eds., Blackwell Science Inc., 1987); and Gene Transfer Vectors for Mammalian Cells (J. M. Miller & M. P. Calos, eds., 1987). Other useful references include Harrison's Principles of Internal Medicine (McGraw Hill; J. Isseleacher et al., eds.), Dubois' Lupus Erythematosus (5th ed.; D. J. Wallace and B. H. Hahn, eds.

### II. Definitions

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

A “subject” can be a vertebrate, a mammal, or a human. Mammals include, but are not limited to, farm animals, sport animals, pets, primates, mice and rats. Subjects also include companion animals including, but not limited to, dogs and cats. In one aspect, a subject is a human.

An “effective amount” or “therapeutically effective amount” refers to an amount of therapeutic compound, such as an oligonucleotide or other anticancer therapy, administered to a subject, either as a single dose or as part of a series of doses, which is effective to produce a desired therapeutic effect.

A “pre-cancer” as used herein refers to having a transformed cell which can evolve or differentiate into a malignant cell. For example, cells transformed by DNA or RNA oncoviruses are pre-cancer cells, and an individual having such transformed cells would be pre-cancerous, or having a pre-cancer. Treating of a pre-cancer by methods described herein involves eliminating or killing such pre-cancer cells before they become cancerous.

A “cancer stem cell” as used herein refers to a self-renewing initiation subpopulation of tumor cells or a small population of cancer cells that are capable of giving rise to new tumors. Cancer stem cells have been identified in a number of cancers including, but not limited to, breast, brain, blood, liver, kidney, cervical, ovarian, colon, and lung cancers among others. See Ponti et al., Cancer Res, 65(13):5506-11, 2005; Feng et al., Oncology Reports, 22:1129-1134, 2009; Zhang et al., Cancer Res, 68(11):4311:4320, 2008; Singh et al., Cancer Res, 63:5821-5828, 2003; Clarke et al., Cancer Res, 66:9339, 2006; Sendurai et al., Cell, 133:704, 2008; Ohata et al., Cancer Res, 72:5101, 2012; and Mukhopapadhyay et al., Plos One, 8(11):e78725, 2013. Methods of treatment as described herein can be used to eliminate such a population of cancer stem cells.

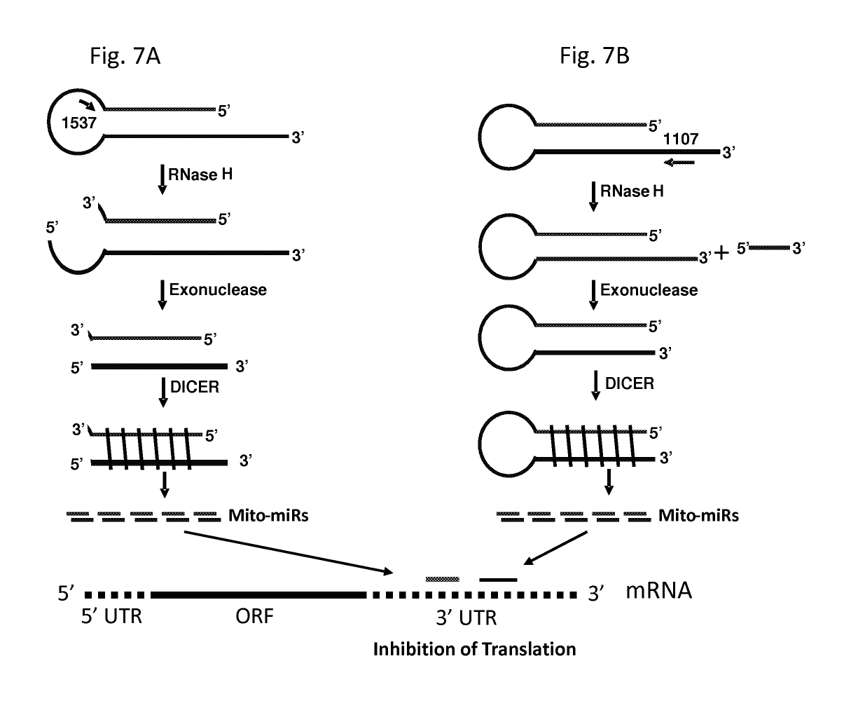

A “microRNA”, “miRNA” or “Mito-miRNA” or “Mito-miR” as used herein is a small RNA molecule, for example 15 to 30 nucleotides per single strand, which can be single stranded or part of a duplex, resulting from the sequential cleavage of ASncmtRNA by RNaseH, exonuclease and Dicer, i.e. the final products of the process steps described in FIGS. 7A and 7B. MicroRNA, or Mito-miRNA can be prepared by the process described in FIGS. 7A and 7B, or can be prepared synthetically. Mito-miRNA as part of duplex typically includes a complementary duplex portion, and non-complementary portions, for example the Dicer processed Mito-miRNA will have an overhanging 3′ end, such that there is a 1-5, also 2-3 nucleotide single stranded region (i.e. overhanging region) on each strand of the duplex.

A “synthetic RNA molecule” or “synthetic RNA” as used herein is an RNA molecule made by chemical synthesis, e.g. by synthetic methods known to one skilled in the art. The RNA molecules provided herein, e.g. the products of the process steps described in FIGS. 7A and 7B, can also be synthetically prepared and isolated for use in the methods as described herein. A synthetic RNA molecule includes an isolated synthetic RNA molecule, and can also be a duplex, i.e. a complementary strand can be similarly synthesized, wherein the two strands need not be an exact complement. For example, synthetic Mito-miRNA can be prepared as part of duplex having a complementary duplex portion, and non-complementary portions, for example the synthetic Mito-miRNA can be made analogous to the Dicer processed Mito-miRNA, i.e. can have an overhanging 3′ end, such that there is a 1-5, also 2-3 nucleotide single stranded region (i.e. overhanging region) on each strand of the duplex.

A “synthetic DNA molecule” or “synthetic DNA” as used herein is a DNA molecule made by chemical synthesis, e.g. by synthetic methods known to one skilled in the art. The DNA molecules provided herein, e.g. DNA molecules analogous to the RNA molecules of the products of the process steps described in FIGS. 7A and 7B, in particular analogous to the Mito-miRNA products of the final step, can also be synthetically prepared and isolated for use in the methods as described herein.

The phrase “sequence analogous to” or “sequence corresponding to”, for example, as it relates to synthetic RNA molecules described herein, indicates that the RNA has a sequence that is identical to or substantially the same as an RNA molecule or analogous DNA molecule as described herein, or as prepared by the methods described herein e.g. the methods as outlined in FIGS. 7A and 7B. In some instances, the synthetic RNA has the identical sequence to the RNA molecules as prepared by the methods described herein. In some instances, the synthetic RNA can have substantially the same sequence, wherein the synthetic RNA can have additional bases on either the 3′ or the 5′ end, or both the 3′ or 5′ end, or can have one or more nucleotides within the sequence that differs from the RNA molecule as prepared by the methods described herein. In some instances, the synthetic RNA is 90%, 91%, 92%, 93%, 94%, 95%, 96%, 97%, 98%, 99%, or 100% identical to the sequence of an isolated RNA as prepared by the methods as described herein. The phrase “sequence identical to” indicates that the RNA has an identical sequence, i.e. the same sequence of bases, although such nucleic acids may have a modified backbone connecting one or more bases in the sequence. As detailed herein, RNA molecules are prepared by certain methods, such as the process described in FIG. 7A or 7B, resulting in RNA molecules of known sequence. As such, RNA molecules can be readily prepared with the identical sequence, or with an analogous sequence (for example, as a DNA sequence) as described herein.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### III. Isolated RNA and Methods of Producing Isolated RNA

Provided herein are isolated RNA molecules, and a method for preparing an isolated RNA molecule, i.e. the method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) optionally digesting the non-coding chimeric mitochondrial RNA molecule cleaved by RNase H with an exonuclease to provide a non-coding chimeric mitochondrial RNA molecule sequentially cleaved by RNase H and exonuclease; (c) optionally digesting the non-coding chimeric mitochondrial RNA molecule sequentially cleaved by RNase H and exonuclease with Dicer to provide a non-coding chimeric mitochondrial RNA molecule sequentially cleaved by RNase H, exonuclease and Dicer; and (d) isolating the non-coding chimeric mitochondrial RNA molecule cleaved by RNase H, and/or optionally isolating the non-coding chimeric mitochondrial RNA molecule sequentially cleaved by RNase H and exonuclease, and/or optionally isolating the non-coding chimeric mitochondrial RNA molecule sequentially cleaved by RNase H, exonuclease and Dicer, to provide the isolated RNA molecule.

In some embodiments of the method for preparing an isolated RNA molecule, the non-coding chimeric mitochondrial RNA molecule is an antisense non-coding chimeric mitochondrial RNA molecule. In some embodiments the antisense non-coding chimeric mitochondrial RNA molecule comprises a RNA sequence corresponding to the sequence selected from the group consisting of SEQ ID NO:4, SEQ ID NO:5 and SEQ ID NO:6.

In another aspect, provided herein is an isolated RNA molecule, wherein said isolated RNA molecule is prepared by the method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) optionally digesting the non-coding chimeric mitochondrial RNA molecule cleaved by RNase H with an exonuclease to provide a non-coding chimeric mitochondrial RNA molecule sequentially cleaved by RNase H and exonuclease; (c) optionally digesting the non-coding chimeric mitochondrial RNA molecule sequentially cleaved by RNase H and exonuclease with Dicer to provide a non-coding chimeric mitochondrial RNA molecule sequentially cleaved by RNase H, exonuclease and Dicer; and isolating the non-coding chimeric mitochondrial RNA molecule cleaved by RNase H, and/or optionally isolating the non-coding chimeric mitochondrial RNA molecule sequentially cleaved by RNase H and exonuclease, and/or optionally isolating the non-coding chimeric mitochondrial RNA molecule sequentially cleaved by RNase H, exonuclease and Dicer, to provide the isolated RNA molecule.

In another aspect, provided herein is an isolated RNA molecule, wherein said isolated RNA molecule comprises a sequence of an RNA molecule selected from the group consisting of a non-coding chimeric mitochondrial RNA molecule cleaved by RNase H; a non-coding chimeric mitochondrial RNA molecule sequentially cleaved by RNase H and exonuclease; and a non-coding chimeric mitochondrial RNA molecule sequentially cleaved by RNase H, exonuclease and Dicer; wherein the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer are prepared by a method comprising (a) annealing one or more oligonucleotides with a non-coding chimeric mitochondrial RNA molecule and digesting with an RNase H to provide the non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide the non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the non-coding chimeric mitochondrial RNA molecule sequentially cleaved by RNase H, exonuclease and Dicer. In some embodiments, said isolated RNA molecule consists essentially of a sequence analogous to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer. In some embodiments, said isolated RNA molecule consists of a sequence analogous to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer. In some embodiments, said isolated RNA molecule comprises a sequence identical to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer. In some embodiments, said isolated RNA molecule consists essentially of a sequence identical to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer. In some embodiments, said isolated RNA molecule consists of a sequence identical to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer.

In another aspect, provided herein is one or more isolated Mito-miRNA molecules comprising a sequence/corresponding to a RNA sequence, selected from the group consisting of 5′-ACAGGCAUGCUCAUAAGGUUAA (SEQ ID NO:7), 5′-UAAACAGGCGGGGUAAGGUUUG (SEQ ID NO:8), 5′AUUGUAGAUAUUGGGCUGUUAA (SEQ ID NO:9), 5′-UUGGGCUGUUAAUUGUCAGUUC (SEQ ID NO:10), 5′-ACAUCACCUCUAGCAUCACCAG (SEQ ID NO:11), 5′-UCCCAAACAUAUAACUGAACU (SEQ ID NO:12), 5′-GUACCCUAACCAUGCGAAAG (SEQ ID NO:13), 5′-CCUCACACCCAAUUGGACCAA (SEQ ID NO:14), 5′-GAGCAGUACAUGCUAAGACUU (SEQ ID NO:15), 5′-CGCCUGCCCAGUGACACAUGU (SEQ ID NO:16), 5′-GGUCUUCUCGUCUUGCUGUGU (SEQ ID NO:17), 5′-UGGCUCUCUCCUUGCAAAGUUAU (SEQ ID NO:18), 5′-ACCUUUGCACGGUUAGGGUAC (SEQ ID NO:19), 5′-AACCUUAUGAGCAUGCCUGU (SEQ ID NO:20), 5′-AACCUUACCCCGCCUGUUUA (SEQ ID NO:21), 5′-AACAGCCCAAUAUCUACAAU (SEQ ID NO:22), 5′-ACUGACAAUUAACAGCCCAA (SEQ ID NO:23), 5′-GGUGAUGCUAGAGGUGAUGU (SEQ ID NO:24), 5′-UUCAGUUAUAUGUUUGGGA (SEQ ID NO:25), 5′-UUCGCAUGGUUAGGGUAC (SEQ ID NO:26), 5′-GGUCCAAUUGGGUGUGAGG (SEQ ID NO:27), 5′-GUCUUAGCAUGUACUGCUC (SEQ ID NO:28), 5′-AUGUGUCACUGGGCAGGCG (SEQ ID NO:29), 5′-ACAGCAAGACGAGAAGACC (SEQ ID NO:30), 5′-AACUUUGCAAGGAGAGAGCCA (SEQ ID NO:31), 5′-ACCCUAACCGUGCAAAGGU (SEQ ID NO:32), 5′-AACCUUAUGAGCAUGCCUGUNN (SEQ ID NO:83), 5′-AACCUUACCCCGCCUGUUUANN (SEQ ID NO:84), 5′-AACAGCCCAAUAUCUACAAUNN (SEQ ID NO:85), 5′-ACUGACAAUUAACAGCCCAANN (SEQ ID NO:86), 5′-GGUGAUGCUAGAGGUGAUGUNN (SEQ ID NO:87), 5′-UUCAGUUAUAUGUUUGGGANN (SEQ ID NO:88), 5′-UUCGCAUGGUUAGGGUACNN (SEQ ID NO:89), 5′-GGUCCAAUUGGGUGUGAGGNN (SEQ ID NO:90), 5′-GUCUUAGCAUGUACUGCUCNN (SEQ ID NO:91), 5′-AUGUGUCACUGGGCAGGCGNN (SEQ ID NO:92), 5′-ACAGCAAGACGAGAAGACCNN (SEQ ID NO:93), 5′-AACUUUGCAAGGAGAGAGCCANN (SEQ ID NO:94), 5′-ACCCUAACCGUGCAAAGGUNN (SEQ ID NO:95), 5′-ACCUUAUGAGCAUGCCUGU (SEQ ID NO:96), 5′-ACCUUACCCCGCCUGUUUA (SEQ ID NO:97), 5′-ACAGCCCAAUAUCUACAAU (SEQ ID NO:98), 5′-CUGACAAUUAACAGCCCAA (SEQ ID NO:99), 5′-GUGAUGCUAGAGGUGAUGU (SEQ ID NO:100), 5′-UCAGUUAUAUGUUUGGGA (SEQ ID NO:101), 5′-UCGCAUGGUUAGGGUAC (SEQ ID NO:102), 5′-GUCCAAUUGGGUGUGAGG (SEQ ID NO:103), 5′-UCUUAGCAUGUACUGCUC (SEQ ID NO: 104), 5′-UGUGUCACUGGGCAGGCG (SEQ ID NO:105), 5′-CAGCAAGACGAGAAGACC (SEQ ID NO:106), 5′-ACUUUGCAAGGAGAGAGCCA (SEQ ID NO:107), 5′-CCCUAACCGUGCAAAGGU (SEQ ID NO: 108), 5′-ACCUUAUGAGCAUGCCUGUNNN (SEQ ID NO:109), 5′-ACCUUACCCCGCCUGUUUANNN (SEQ ID NO:110), 5′-ACAGCCCAAUAUCUACAAUNNN (SEQ ID NO:111), 5′-CUGACAAUUAACAGCCCAANNN (SEQ ID NO:112), 5′-GUGAUGCUAGAGGUGAUGUNNN (SEQ ID NO: 113), 5′-UCAGUUAUAUGUUUGGGANNN (SEQ ID NO:114), 5′-UCGCAUGGUUAGGGUACNNN (SEQ ID NO: 115), 5′-GUCCAAUUGGGUGUGAGGNNN (SEQ ID NO:116), 5′-UCUUAGCAUGUACUGCUCNNN (SEQ ID NO: 117), 5′-UGUGUCACUGGGCAGGCGNNN (SEQ ID NO: 118), 5′-CAGCAAGACGAGAAGACCNNN (SEQ ID NO:119), 5′-ACUUUGCAAGGAGAGAGCCANNN (SEQ ID NO: 120), and 5′-CCCUAACCGUGCAAAGGUNNN (SEQ ID NO: 121), wherein N represents any nucleotide base, where in some embodiments, each N is U. In some embodiments, the isolated Mito-miRNA molecule comprises an isolated double stranded Mito-miRNA molecule wherein one of the strands comprises a sequence/corresponds to a RNA sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121. In some embodiments, the isolated Mito-miRNA molecule is an isolated double stranded Mito-miRNA molecule, wherein one strand of the double stranded RNA molecule consists of a sequence/corresponds to a RNA sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. In some embodiments, the isolated Mito-miRNA molecule is an isolated double stranded Mito-miRNA molecule selected from the group consisting of one strand having the sequence of SEQ ID NO:7 and the other strand having the sequence of SEQ ID NO:83 or SEQ ID NO: 109, one strand having the sequence of SEQ ID NO:8 and the other strand having the sequence of SEQ ID NO:84 or SEQ ID NO: 110, one strand having the sequence of SEQ ID NO:9 and the other strand having the sequence of SEQ ID NO:85 or SEQ ID NO:111, one strand having the sequence of SEQ ID NO: 10 and the other strand having the sequence of SEQ ID NO:86 or SEQ ID NO: 112, one strand having the sequence of SEQ ID NO: 11 and the other strand having the sequence of SEQ ID NO:87 or SEQ ID NO: 113, one strand having the sequence of SEQ ID NO: 12 and the other strand having the sequence of SEQ ID NO:88 or SEQ ID NO: 114, one strand having the sequence of SEQ ID NO: 13 and the other strand having the sequence of SEQ ID NO:89 or SEQ ID NO: 115, one strand having the sequence of SEQ ID NO: 14 and the other strand having the sequence of SEQ ID NO:90 or SEQ ID NO: 116, one strand having the sequence of SEQ ID NO: 15 and the other strand having the sequence of SEQ ID NO:91 or SEQ ID NO: 117, one strand having the sequence of SEQ ID NO: 16 and the other strand having the sequence of SEQ ID NO:92 or SEQ ID NO: 118, one strand having the sequence of SEQ ID NO: 17 and the other strand having the sequence of SEQ ID NO:93 or SEQ ID NO: 119, one strand having the sequence of SEQ ID NO:18 and the other strand having the sequence of SEQ ID NO:94 or SEQ ID NO: 120, and one strand having the sequence of SEQ ID NO: 19 and the other strand having the sequence of SEQ ID NO:95 or SEQ ID NO:121.

In another aspect, provided herein are one or more synthetic Mito-miRNA molecules comprising a sequence selected from the group consisting of 5′-ACAGGCAUGCUCAUAAGGUUAA (SEQ ID NO:7), 5′-UAAACAGGCGGGGUAAGGUUUG (SEQ ID NO:8), 5′AUUGUAGAUAUUGGGCUGUUAA (SEQ ID NO:9), 5′-UUGGGCUGUUAAUUGUCAGUUC (SEQ ID NO:10), 5′-ACAUCACCUCUAGCAUCACCAG (SEQ ID NO:11), 5′-UCCCAAACAUAUAACUGAACU (SEQ ID NO:12), 5′-GUACCCUAACCAUGCGAAAG (SEQ ID NO:13), 5′-CCUCACACCCAAUUGGACCAA (SEQ ID NO:14), 5′-GAGCAGUACAUGCUAAGACUU (SEQ ID NO:15), 5′-CGCCUGCCCAGUGACACAUGU (SEQ ID NO:16), 5′-GGUCUUCUCGUCUUGCUGUGU (SEQ ID NO:17), 5′-UGGCUCUCUCCUUGCAAAGUUAU (SEQ ID NO:18), 5′-ACCUUUGCACGGUUAGGGUAC (SEQ ID NO:19), 5′-AACCUUAUGAGCAUGCCUGU (SEQ ID NO:20), 5′-AACCUUACCCCGCCUGUUUA (SEQ ID NO:21), 5′-AACAGCCCAAUAUCUACAAU (SEQ ID NO:22), 5′-ACUGACAAUUAACAGCCCAA (SEQ ID NO:23), 5′-GGUGAUGCUAGAGGUGAUGU (SEQ ID NO:24), 5′-UUCAGUUAUAUGUUUGGGA (SEQ ID NO:25), 5′-UUCGCAUGGUUAGGGUAC (SEQ ID NO:26), 5′-GGUCCAAUUGGGUGUGAGG (SEQ ID NO:27), 5′-GUCUUAGCAUGUACUGCUC (SEQ ID NO:28), 5′-AUGUGUCACUGGGCAGGCG (SEQ ID NO:29), 5′-ACAGCAAGACGAGAAGACC (SEQ ID NO:30), 5′-AACUUUGCAAGGAGAGAGCCA (SEQ ID NO:31), 5′-ACCCUAACCGUGCAAAGGU (SEQ ID NO:32), 5′-AACCUUAUGAGCAUGCCUGUNN (SEQ ID NO:83), 5′-AACCUUACCCCGCCUGUUUANN (SEQ ID NO:84), 5′-AACAGCCCAAUAUCUACAAUNN (SEQ ID NO:85), 5′-ACUGACAAUUAACAGCCCAANN (SEQ ID NO:86), 5′-GGUGAUGCUAGAGGUGAUGUNN (SEQ ID NO:87), 5′-UUCAGUUAUAUGUUUGGGANN (SEQ ID NO:88), 5′-UUCGCAUGGUUAGGGUACNN (SEQ ID NO:89), 5′-GGUCCAAUUGGGUGUGAGGNN (SEQ ID NO:90), 5′-GUCUUAGCAUGUACUGCUCNN (SEQ ID NO:91), 5′-AUGUGUCACUGGGCAGGCGNN (SEQ ID NO:92), 5′-ACAGCAAGACGAGAAGACCNN (SEQ ID NO:93), 5′-AACUUUGCAAGGAGAGAGCCANN (SEQ ID NO:94), 5′-ACCCUAACCGUGCAAAGGUNN (SEQ ID NO:95), 5′-ACCUUAUGAGCAUGCCUGU (SEQ ID NO:96), 5′-ACCUUACCCCGCCUGUUUA (SEQ ID NO:97), 5′-ACAGCCCAAUAUCUACAAU (SEQ ID NO:98), 5′-CUGACAAUUAACAGCCCAA (SEQ ID NO:99), 5′-GUGAUGCUAGAGGUGAUGU (SEQ ID NO:100), 5′-UCAGUUAUAUGUUUGGGA (SEQ ID NO:101), 5′-UCGCAUGGUUAGGGUAC (SEQ ID NO:102), 5′-GUCCAAUUGGGUGUGAGG (SEQ ID NO:103), 5′-UCUUAGCAUGUACUGCUC (SEQ ID NO: 104), 5′-UGUGUCACUGGGCAGGCG (SEQ ID NO:105), 5′-CAGCAAGACGAGAAGACC (SEQ ID NO:106), 5′-ACUUUGCAAGGAGAGAGCCA (SEQ ID NO:107), 5′-CCCUAACCGUGCAAAGGU (SEQ ID NO: 108), 5′-ACCUUAUGAGCAUGCCUGUNNN (SEQ ID NO:109), 5′-ACCUUACCCCGCCUGUUUANNN (SEQ ID NO:110), 5′-ACAGCCCAAUAUCUACAAUNNN (SEQ ID NO: 111), 5′-CUGACAAUUAACAGCCCAANNN (SEQ ID NO:112), 5′-GUGAUGCUAGAGGUGAUGUNNN (SEQ ID NO:113), 5′-UCAGUUAUAUGUUUGGGANNN (SEQ ID NO:114), 5′-UCGCAUGGUUAGGGUACNNN (SEQ ID NO:115), 5′-GUCCAAUUGGGUGUGAGGNNN (SEQ ID NO:116), 5′-UCUUAGCAUGUACUGCUCNNN (SEQ ID NO:117), 5′-UGUGUCACUGGGCAGGCGNNN (SEQ ID NO:118), 5′-CAGCAAGACGAGAAGACCNNN (SEQ ID NO:119), 5′-ACUUUGCAAGGAGAGAGCCANNN (SEQ ID NO: 120), and 5′-CCCUAACCGUGCAAAGGUNNN (SEQ ID NO: 121), wherein N represents any nucleotide base, where in some embodiments, each N is U. In some embodiments, the synthetic Mito-miRNA molecule comprises a synthetic double stranded Mito-miRNA molecule wherein one of the strands comprises a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO: 121. In some embodiments, the synthetic Mito-miRNA molecule is a synthetic double stranded Mito-miRNA molecule, wherein one strand of the double stranded Mito-miRNA molecule consists of a sequence/corresponds to a RNA selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. In some embodiments, the synthetic Mito-miRNA molecule is a synthetic double stranded Mito-miRNA molecule selected from the group consisting of one strand having the sequence of SEQ ID NO:7 and the other strand having the sequence of SEQ ID NO:83 or SEQ ID NO: 109, one strand having the sequence of SEQ ID NO:8 and the other strand having the sequence of SEQ ID NO:84 or SEQ ID NO: 110, one strand having the sequence of SEQ ID NO:9 and the other strand having the sequence of SEQ ID NO:85 or SEQ ID NO: 111, one strand having the sequence of SEQ ID NO: 10 and the other strand having the sequence of SEQ ID NO:86 or SEQ ID NO: 112, one strand having the sequence of SEQ ID NO: 11 and the other strand having the sequence of SEQ ID NO:87 or SEQ ID NO: 113, one strand having the sequence of SEQ ID NO: 12 and the other strand having the sequence of SEQ ID NO:88 or SEQ ID NO: 114, one strand having the sequence of SEQ ID NO: 13 and the other strand having the sequence of SEQ ID NO:89 or SEQ ID NO: 115, one strand having the sequence of SEQ ID NO: 14 and the other strand having the sequence of SEQ ID NO:90 or SEQ ID NO: 116, one strand having the sequence of SEQ ID NO: 15 and the other strand having the sequence of SEQ ID NO:91 or SEQ ID NO: 117, one strand having the sequence of SEQ ID NO: 16 and the other strand having the sequence of SEQ ID NO:92 or SEQ ID NO: 118, one strand having the sequence of SEQ ID NO: 17 and the other strand having the sequence of SEQ ID NO:93 or SEQ ID NO: 119, one strand having the sequence of SEQ ID NO: 18 and the other strand having the sequence of SEQ ID NO:94 or SEQ ID NO: 120, and one strand having the sequence of SEQ ID NO:19 and the other strand having the sequence of SEQ ID NO:95 or SEQ ID NO:121.

In another aspect, provided herein are one or more synthetic DNA molecules analogous to Mito-miRNA molecules. In one embodiment, one or more synthetic DNA molecules are provided, comprising a sequence selected from the group consisting of 5′-ACAGGCATGCTCATAAGGTTAA (SEQ ID NO:57), 5′-TAAACAGGCGGGGTAAGGTTTG (SEQ ID NO:58), 5′ATTGTAGATATTGGGCTGTTAA (SEQ ID NO:59), 5′-TTGGGCTGTTAATTGTCAGTTC (SEQ ID NO:60), 5′-ACATCACCTCTAGCATCACCAG (SEQ ID NO:61), 5′-TCCCAAACATATAACTGAACT (SEQ ID NO:62), 5′-GTACCCTAACCATGCGAAAG (SEQ ID NO:63), 5′-CCTCACACCCAATTGGACCAA (SEQ ID NO:64), 5′-GAGCAGTACATGCTAAGACTT (SEQ ID NO:65), 5′-CGCCTGCCCAGTGACACATGT (SEQ ID NO:66), 5′-GGTCTTCTCGTCTTGCTGTGT (SEQ ID NO:67), 5′-TGGCTCTCTCCTTGCAAAGTTAT (SEQ ID NO:68), 5′-ACCTTTGCACGGTTAGGGTAC (SEQ ID NO:69), 5′-AACCTTATGAGCATGCCTGT (SEQ ID NO:70), 5′-AACCTTACCCCGCCTGTTTA (SEQ ID NO:71), 5′-AACAGCCCAATATCTACAAT (SEQ ID NO:72), 5′-ACTGACAATTAACAGCCCAA (SEQ ID NO:73), 5′-GGTGATGCTAGAGGTGATGT (SEQ ID NO:74), 5′-TTCAGTTATATGTTTGGGA (SEQ ID NO:75), 5′-TTCGCATGGTTAGGGTAC (SEQ ID NO:76), 5′-GGTCCAATTGGGTGTGAGG (SEQ ID NO:77), 5′-GTCTTAGCATGTACTGCTC (SEQ ID NO:78), 5′-ATGTGTCACTGGGCAGGCG (SEQ ID NO:79), 5′-ACAGCAAGACGAGAAGACC (SEQ ID NO:80), 5′-AACTTTGCAAGGAGAGAGCCA (SEQ ID NO:81), 5′-ACCCTAACCGTGCAAAGGT (SEQ ID NO:82), 5′-AACCTTATGAGCATGCCTGTNN (SEQ ID NO: 122), 5′-AACCTTACCCCGCCTGTTTANN (SEQ ID NO: 123), 5′-AACAGCCCAATATCTACAATNN (SEQ ID NO: 124), 5′-ACTGACAATTAACAGCCCAANN (SEQ ID NO: 125), 5′-GGTGATGCTAGAGGTGATGTNN (SEQ ID NO: 126), 5′-TTCAGTTATATGTTTGGGANN (SEQ ID NO: 127), 5′-TTCGCATGGTTAGGGTACNN (SEQ ID NO: 128), 5′-GGTCCAATTGGGTGTGAGGNN (SEQ ID NO: 129), 5′-GTCTTAGCATGTACTGCTCNN (SEQ ID NO:130), 5′-ATGTGTCACTGGGCAGGCGNN (SEQ ID NO:131), 5′-ACAGCAAGACGAGAAGACCNN (SEQ ID NO:132), 5′-AACTTTGCAAGGAGAGAGCCANN (SEQ ID NO: 133), 5′-ACCCTAACCGTGCAAAGGTNN (SEQ ID NO:134), 5′-ACCTTATGAGCATGCCTGT (SEQ ID NO: 135), 5′-ACCTTACCCCGCCTGTTTA (SEQ ID NO:136), 5′-ACAGCCCAATATCTACAAT (SEQ ID NO:137), 5′-CTGACAATTAACAGCCCAA (SEQ ID NO: 138), 5′-GTGATGCTAGAGGTGATGT (SEQ ID NO: 139), 5′-TCAGTTATATGTTTGGGA (SEQ ID NO: 140), 5′-TCGCATGGTTAGGGTAC (SEQ ID NO: 141), 5′-GTCCAATTGGGTGTGAGG (SEQ ID NO: 142), 5′-TCTTAGCATGTACTGCTC (SEQ ID NO: 143), 5′-TGTGTCACTGGGCAGGCG (SEQ ID NO: 144), 5′-CAGCAAGACGAGAAGACC (SEQ ID NO: 145), 5′-ACTTTGCAAGGAGAGAGCCA (SEQ ID NO: 146), 5′-CCCTAACCGTGCAAAGGT (SEQ ID NO: 147), 5′-ACCTTATGAGCATGCCTGTNNN (SEQ ID NO: 148), 5′-ACCTTACCCCGCCTGTTTANNN (SEQ ID NO: 149), 5′-ACAGCCCAATATCTACAATNNN (SEQ ID NO:150), 5′-CTGACAATTAACAGCCCAANNN (SEQ ID NO:151), 5′-GTGATGCTAGAGGTGATGTNNN (SEQ ID NO:152), 5′-TCAGTTATATGTTTGGGANNN (SEQ ID NO: 153), 5′-TCGCATGGTTAGGGTACNNN (SEQ ID NO:154), 5′-GTCCAATTGGGTGTGAGGNNN (SEQ ID NO: 155), 5′-TCTTAGCATGTACTGCTCNNN (SEQ ID NO:156), 5′-TGTGTCACTGGGCAGGCGNNN (SEQ ID NO:157), 5′-CAGCAAGACGAGAAGACCNNN (SEQ ID NO: 158), 5′-ACTTTGCAAGGAGAGAGCCANNN (SEQ ID NO:159), and 5′-CCCTAACCGTGCAAAGGTNNN (SEQ ID NO:160), wherein N represents any nucleotide base, where in some embodiments, each N is T. In some embodiments, the synthetic DNA molecule comprises a synthetic double stranded DNA molecule wherein one of the strands comprises a sequence selected from the group consisting of SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO: 140, SEQ ID NO:141, SEQ ID NO: 142, SEQ ID NO: 143, SEQ ID NO:144, SEQ ID NO:145, SEQ ID NO:146, SEQ ID NO:147, SEQ ID NO:148, SEQ ID NO: 149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO: 159, and SEQ ID NO: 160. In some embodiments, the synthetic DNA molecule is a synthetic double stranded DNA molecule, wherein one strand of the double stranded DNA molecule consists of a sequence selected from the group consisting of SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, and SEQ ID NO:69. In some embodiments, the synthetic DNA molecule is a synthetic double stranded DNA molecule selected from the group consisting of one strand having the sequence of SEQ ID NO:57 and the other strand having the sequence of SEQ ID NO: 122 or SEQ ID NO: 148, one strand having the sequence of SEQ ID NO:58 and the other strand having the sequence of SEQ ID NO: 123 or SEQ ID NO: 149, one strand having the sequence of SEQ ID NO:59 and the other strand having the sequence of SEQ ID NO: 124 or SEQ ID NO: 150, one strand having the sequence of SEQ ID NO:60 and the other strand having the sequence of SEQ ID NO: 125 or SEQ ID NO:151, one strand having the sequence of SEQ ID NO:61 and the other strand having the sequence of SEQ ID NO: 126 or SEQ ID NO: 152, one strand having the sequence of SEQ ID NO:62 and the other strand having the sequence of SEQ ID NO: 127 or SEQ ID NO: 153, one strand having the sequence of SEQ ID NO:63 and the other strand having the sequence of SEQ ID NO: 128 or SEQ ID NO: 154, one strand having the sequence of SEQ ID NO:64 and the other strand having the sequence of SEQ ID NO: 129 or SEQ ID NO: 155, one strand having the sequence of SEQ ID NO:65 and the other strand having the sequence of SEQ ID NO: 130 or SEQ ID NO: 156, one strand having the sequence of SEQ ID NO:66 and the other strand having the sequence of SEQ ID NO:131 or SEQ ID NO:157, one strand having the sequence of SEQ ID NO:67 and the other strand having the sequence of SEQ ID NO: 132 or SEQ ID NO: 158, one strand having the sequence of SEQ ID NO:68 and the other strand having the sequence of SEQ ID NO: 133 or SEQ ID NO: 159, and one strand having the sequence of SEQ ID NO:69 and the other strand having the sequence of SEQ ID NO:134 or SEQ ID NO:160.

The treatment of cancer cells with oligonucleotides complementary to an antisense non-coding chimeric mitochondrial RNA (ASncmtRNA) has been described, for example, in U.S. Pat. No. 8,318,686, the disclosure of which is hereby incorporated by reference herein in its entirety, in particular with respect to the antisense non-coding chimeric mitochondrial RNA and the oligonucleotides complementary to the antisense non-coding chimeric mitochondrial RNA. While such oligonucleotides are effective as a possible cancer therapy, it has been found that a number of RNA molecules that act downstream of the mechanism of action of these oligonucleotides may provide a more efficient therapeutic molecule for the treatment of cancer. The general process that takes place when the oligonucleotides as described herein and in U.S. Pat. No. 8,318,686 was not known, and the discovery of this process provides for the isolation of useful RNA molecules as described herein. The general process is shown in FIGS. 7A and 7B. The oligonucleotides complementary to the ASncmtRNA are annealed to the complementary region on ASncmtRNA, either in the loop regions (FIG. 7A) or the 3′ stem region (FIG. 7B), which provides a substrate for Ribonuclease H (RNase H) cleavage. The RNase H cleaved product can be isolated for use in treating cancers, or are further processed by 5′ exonuclease cleavage (e.g. where RNase H cleaves in the loop region) or 3′ exonuclease cleavage (e.g. where RNase H cleaves in the 3′ stem region). The resulting product of exonuclease cleavage can be isolated for use in treating cancers, or are further processed by cleavage with Dicer enzyme. The Dicer cleaved product results in mitochondrial microRNA molecules, or Mito-miRNA. The Mito-miRNA can be isolated and used for treating cancers. As any of the isolated RNA molecules described herein will be processed by this pathway, the resulting Mito-miRNA acts on tumor cells, resulting in cell death. This activity is shown to be exquisitely selective to a variety of tumor cells, such that the Mito-miRNAs provide an ideal therapeutic for the treatment of cancers. The mito-miRNA or the DNA analogous to the mito-miRNA can be prepared synthetically, and can be used for treating cancers as described herein.

A. Human Non-Coding Chimeric Mitochondrial RNAs (ncmtRNAs)

Human cells express a number of unique chimeric mitochondrial RNA molecules. These molecules are non-coding (i.e., they are not known to serve as a template for the translation of a protein) and comprise the 16S mitochondrial ribosomal RNA covalently linked at the 5′ end to an inverted repeat sequence. Non-coding chimeric mitochondrial RNA molecules are found in two forms: sense and antisense.

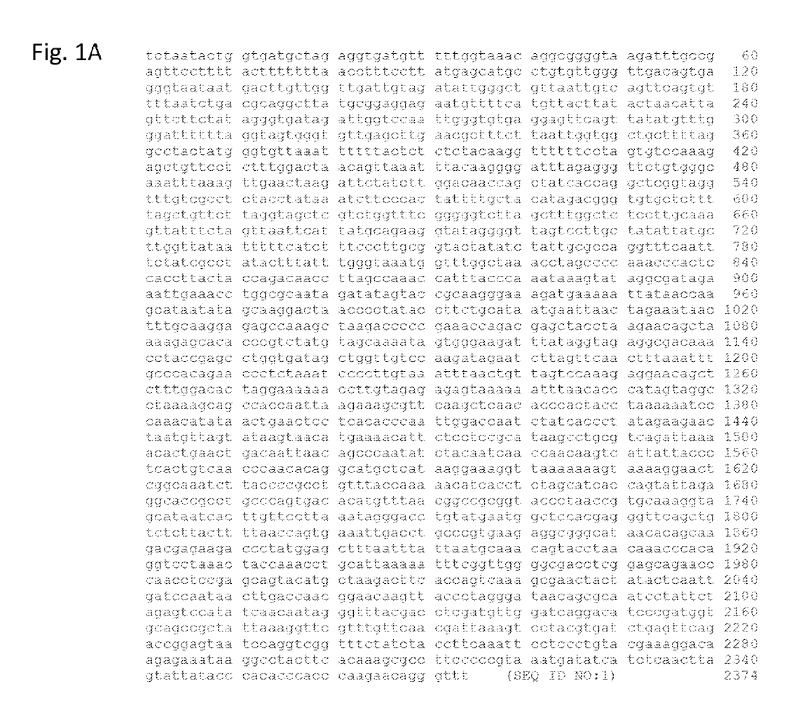

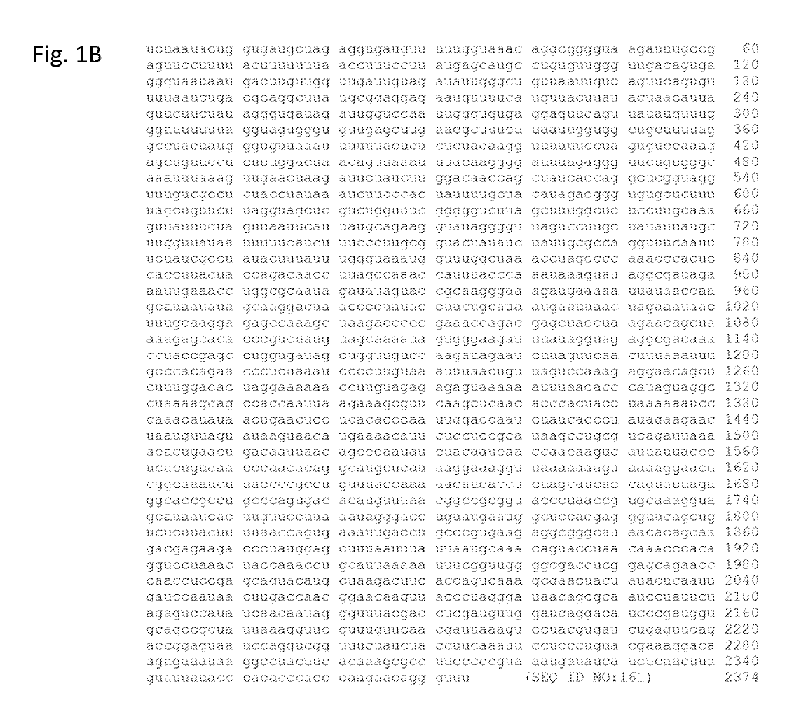

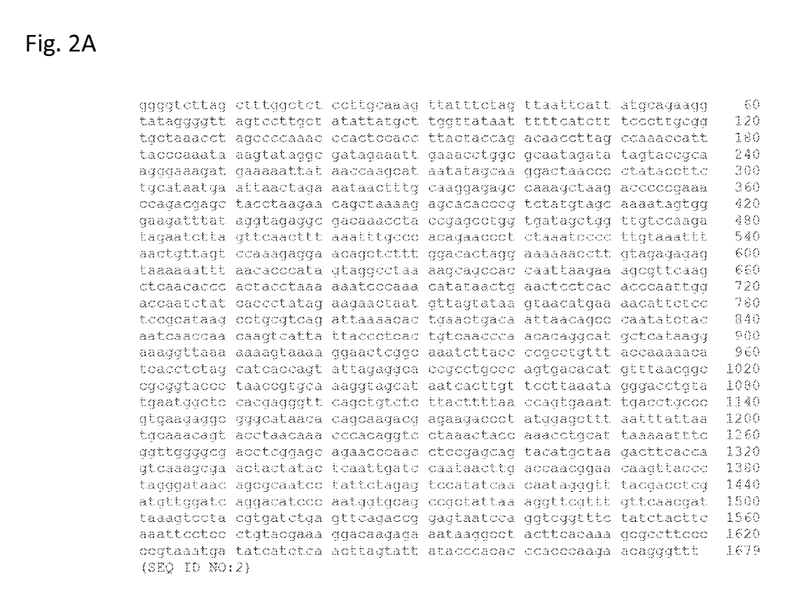

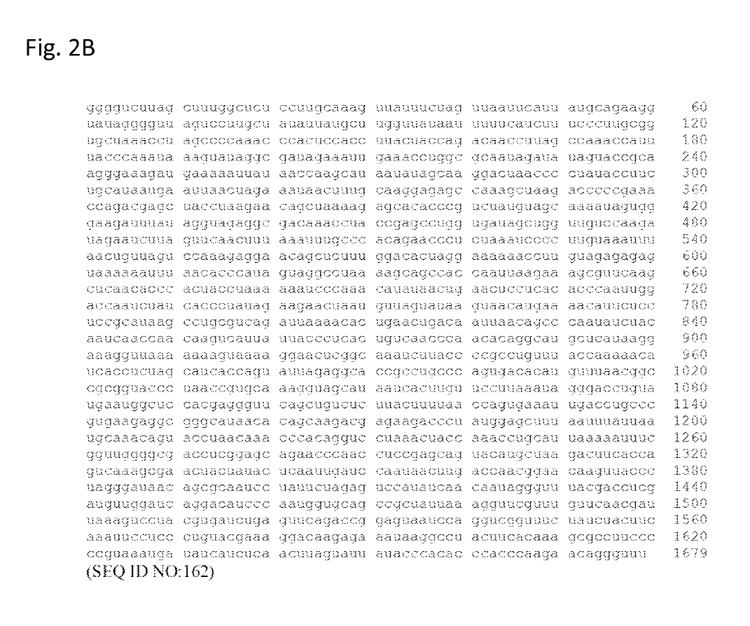

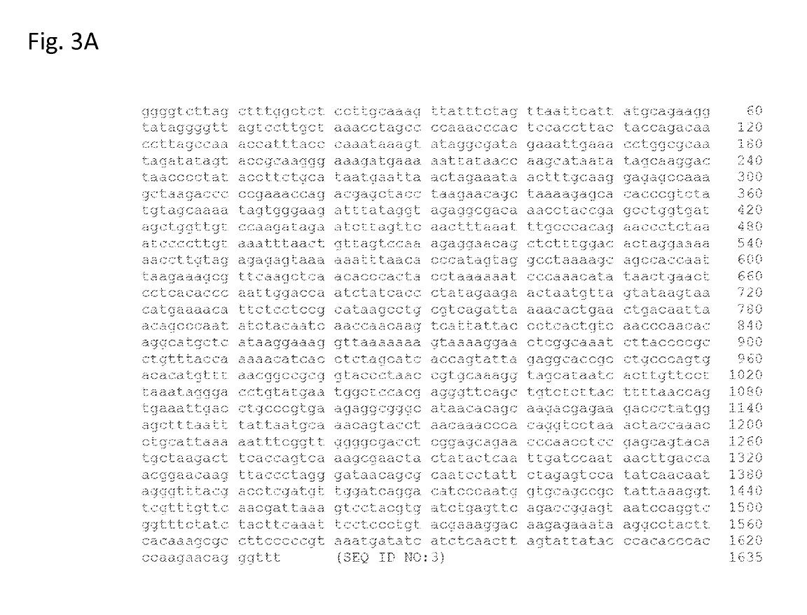

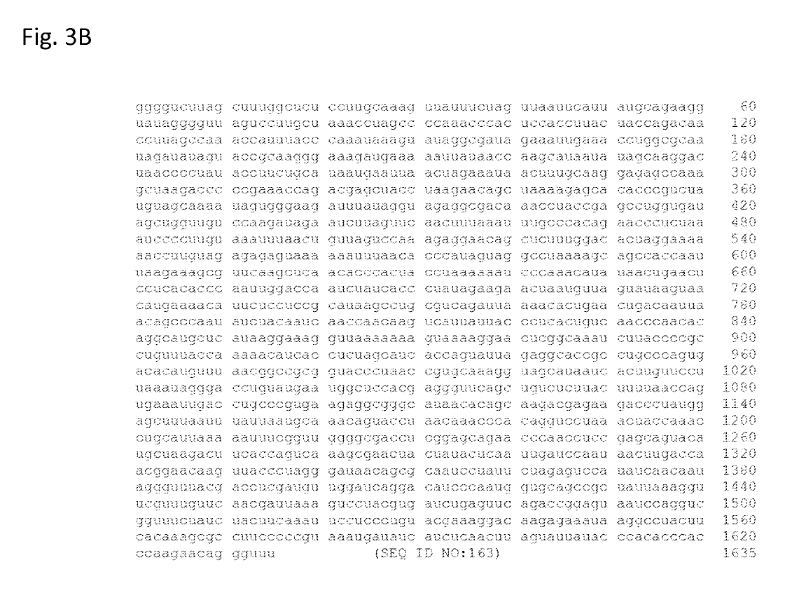

The sense non-coding chimeric mitochondrial RNA (SncmtRNA) molecule corresponds to the 16S mitochondrial ribosomal RNA transcribed from the “H-strand” of the circular mitochondrial genome. Covalently linked to the 5′ end of this RNA molecule is a nucleotide sequence corresponding to the inverted repeat sequence of the 16S mitochondrial ribosomal RNA, transcribed from the “L-strand” of the mitochondrial genome. The size of the inverted repeat sequence in the SncmtRNA can vary from about 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, or 800 nucleotides or more to between about 100-200, 150-250, 200-300, 250-350, 400-500, 450-550, 500-600, 550-650, 600-700, 650-750, or 700-800 nucleotides or more, including any number in between these values. In one embodiment, the inverted repeat sequence in the SncmtRNA corresponds to a fragment of 815 nucleotides of the RNA transcribed from the L-strand of the 16S gene of the mitochondrial genome. In another embodiment, the SncmtRNA comprises a sequence corresponding to SEQ ID NO:1, SEQ ID NO:2, or SEQ ID NO:3 as provided in FIG. 1A, FIG. 2A, and FIG. 3A, respectively, or the SncmtRNA comprises a sequence selected from the group consisting of SEQ ID NO:161, SEQ ID NO:162, and SEQ ID NO:163 as provided in FIG. 1B, FIG. 2B, and FIG. 3B, respectively.

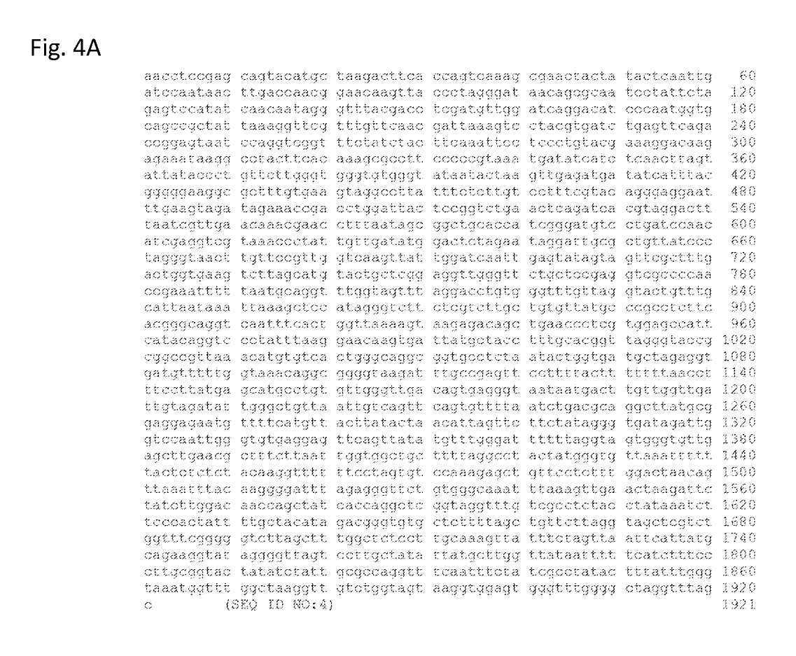

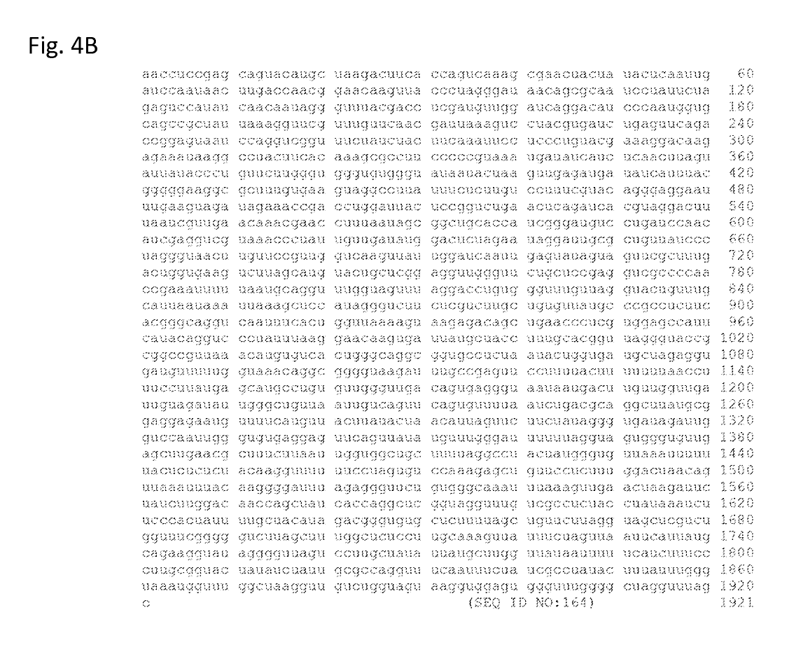

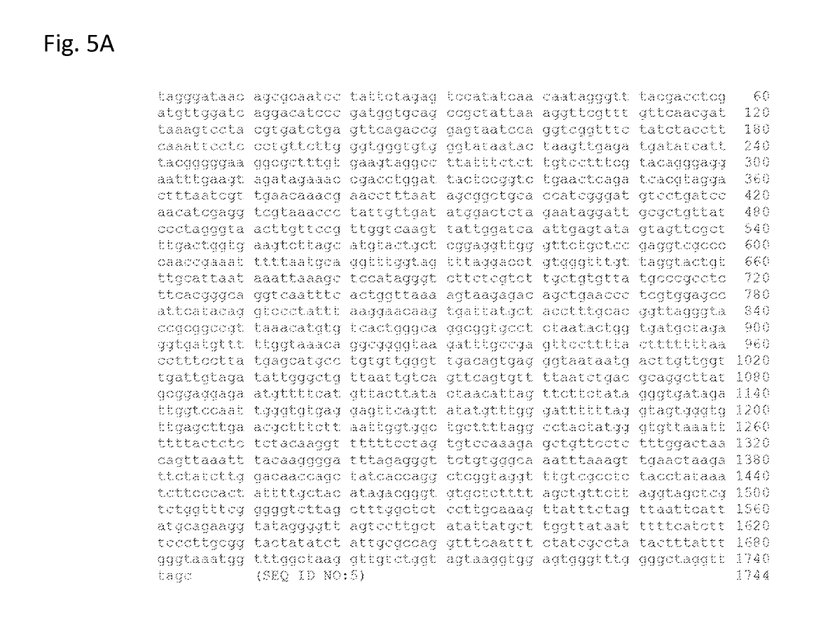

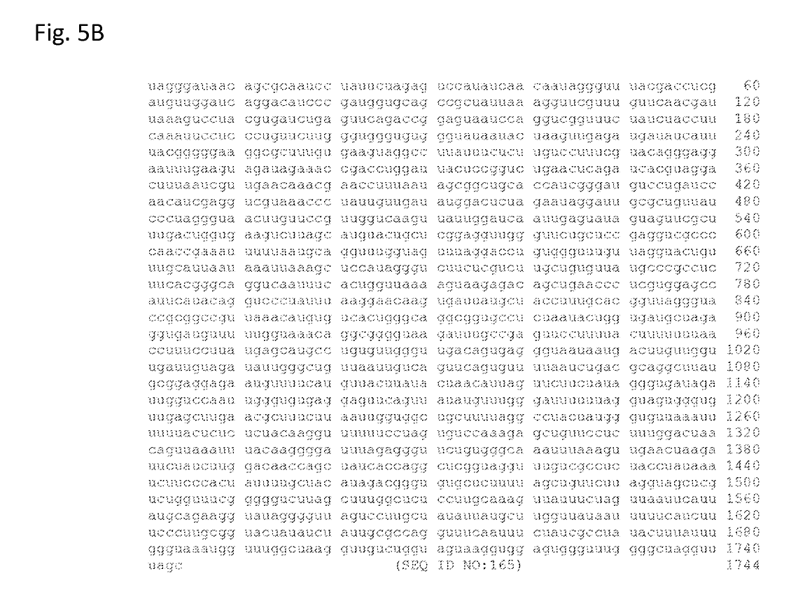

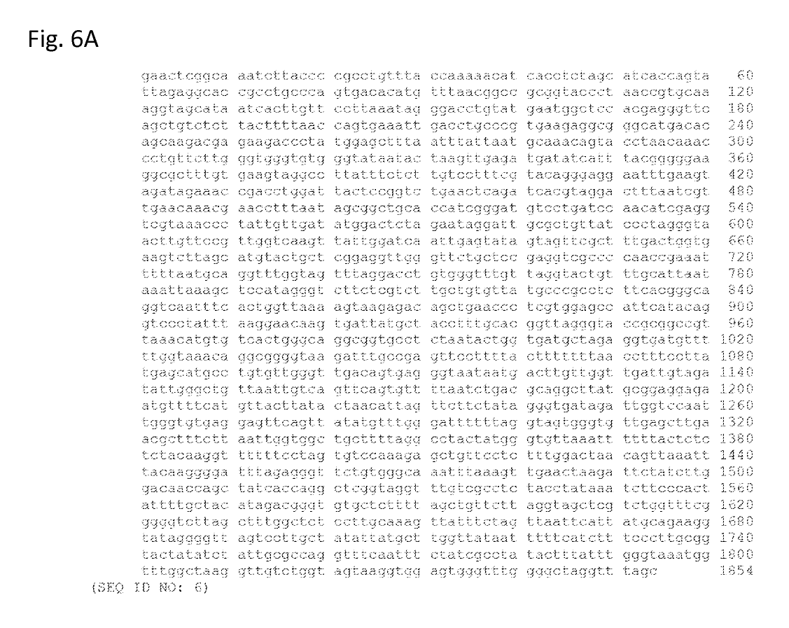

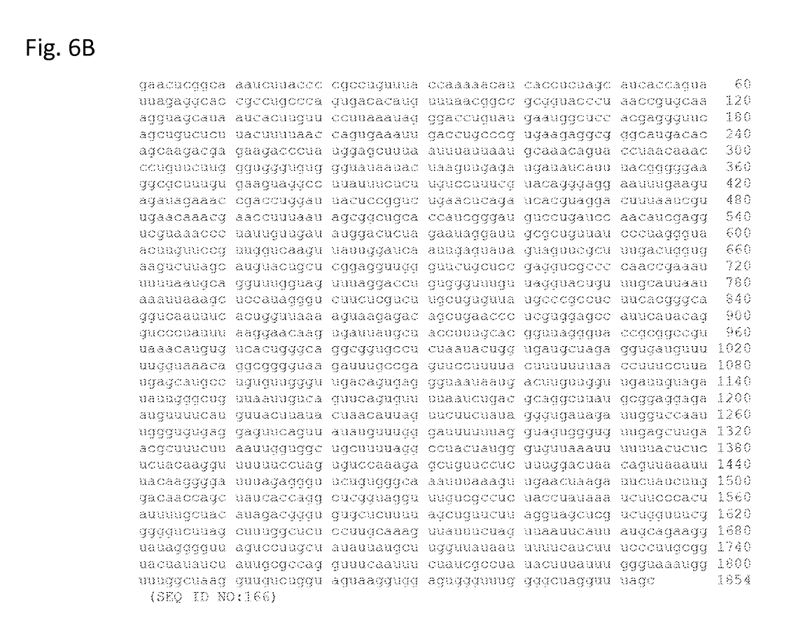

The antisense non-coding chimeric mitochondrial RNA (ASncmtRNA) molecule corresponds to the 16S mitochondrial ribosomal RNA transcribed from the “L-strand” of the circular mitochondrial genome. Covalently linked to the 5′ end of this RNA molecule is a nucleotide sequence corresponding to the inverted repeat sequence of the 16S mitochondrial ribosomal RNA gene, transcribed from the “H-strand” of the mitochondrial genome. The size of the inverted repeat sequence in the ASncmtRNA can vary from about 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800 nucleotides or more to between about 100-200, 150-250, 200-300, 250-350, 400-500, 450-550, 500-600, 550-650, 600-700, 650-750, or 700-800 or more, including any number in between these values. In another embodiment, the ASncmtRNA comprises a sequence corresponding to SEQ ID NO:4, SEQ ID NO:5, or SEQ ID NO:6 as provided in FIG. 4A, FIG. 5A, and FIG. 6A, respectively, or the ASncmtRNA comprises a sequence selected from the group consisting of SEQ ID NO: 164, SEQ ID NO:165, and SEQ ID NO:166 as provided in FIG. 4B, FIG. 5B, and FIG. 6B, respectively.

Further information related to non-coding chimeric mitochondrial RNA molecules can be found in U.S. Pat. No. 8,318,686.

B. Oligonucleotides Complementary to ASncmtRNAs

The method involves the use of oligonucleotides complementary to an antisense non-coding chimeric mitochondrial RNA (ASncmtRNA) molecule as described herein or in U.S. Pat. No. 8,318,686. The first step of the process outlined in FIGS. 7A and 7B involves annealing an oligonucleotide complementary to an ASncmtRNA molecule. As used herein, an oligonucleotide sequence is “complementary” to a portion of an ASncmtRNA, as referred to herein, if the oligonucleotide possesses a sequence having sufficient complementarity to be able to hybridize with the ASncmtRNA to form a stable duplex. The ability to hybridize will depend on both the degree of complementarity and the length of the oligonucleotide. Generally, the longer the hybridizing oligonucleotide, the more base mismatches with an ASncmtRNA it may contain and still form a stable duplex. In some aspects, the oligonucleotides used as in the preparation of an isolated RNA molecule for use as an anticancer therapy according to the methods disclosed herein are at least 8 (such as at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more) bases in length. Those skilled in the art can ascertain a tolerable degree of mismatch by use of standard procedures to determine the melting point of the hybridized complex. In some embodiments, the oligonucleotides are at least 85% (such as at least 86%, 87%, 88%, 89%, 90%, 91%, 92%, 93%, 94%, 95%, 96%, 97%, 98%, 99%, or 100%) complementary to the ASncmtRNA molecule. In some embodiments, the complementary oligonucleotide is an antisense oligonucleotide. In one embodiment, the oligonucleotides are complementary to the ASncmtRNAs corresponding to one or more of SEQ ID NO:4, SEQ ID NO:5, or SEQ ID NO:6. In another embodiment, the oligonucleotideis an oligonucleotide as disclosed in U.S. Pat. No. 8,318,686. In some embodiments, the oligonucleotide is selected from the group consisting of 126S (5′-AGATGAAAAATTATAACCAA SEQ ID NO:33), 226S (5′-TAAGACCCCCGAAACCAGAC SEQ ID NO:45), 552S (5′-TACCTAAAAAATCCCAAACA SEQ ID NO:34), 1107S (5′-GTCCTAAACTACCAAACC SEQ ID NO:35), and 1537S (5′-CACCCACCCAAGAACAGG SEQ ID NO:36).

C. Methods for Producing RNA from ASncmtRNA Cleaved by RNase H.

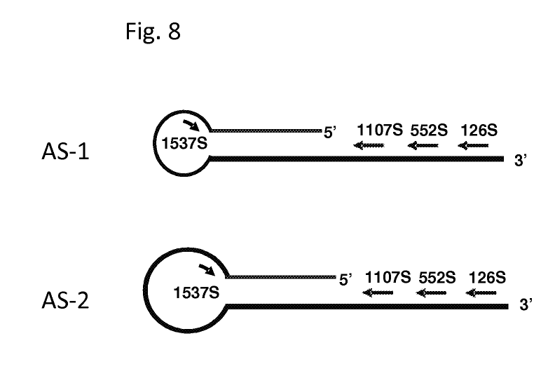

The methods disclosed herein for producing an isolated RNA molecule as described herein involve the RNase H cleavage of an ASncmtRNA. The method involves the use of oligonucleotides complementary to an antisense non-coding chimeric mitochondrial RNA (ASncmtRNA) molecule as described herein or in U.S. Pat. No. 8,318,686. The oligonucleotides can be complementary to any region of the ASncmtRNA, including the 3′ single stranded stem region or the loop region, as shown in FIG. 8.

The process described in FIGS. 7A and 7B that occurs in cells can be used and the desired RNA molecules can be isolated from cells. Alternatively, the process can be replicated without the use of cells, in vitro, as the oligonucleotides can be annealed with ASncmtRNA in a suitable buffer and combined with RNase H, and reacted under suitable conditions to produce the ASncmtRNA cleaved by RNase H. RNase is readily available (e.g. New England BioLabs catalog # M0297S or M0297L; MP Biomedicals catalog #152025; Life Technologies catalog #18021-014) and can be used according to the supplier's direction, or modifications thereof known to one skilled in the art. The resulting RNA molecule can be isolated and used in the methods as described herein. It is also possible to prepare the desired RNA synthetically by methods known to one skilled in the art.

D. Methods for Producing RNA from ASncmtRNA Sequentially Cleaved by RNase and Exonuclease.

The RNase H cleaved RNA molecule can be further processed without cells, in vitro, by the reaction described in FIGS. 7A and 7B. The reaction solution from the RNase H cleavage can be suitably adjusted and a suitable exonuclease can be added, and then reacted under suitable conditions to produce the ASncmtRNA sequentially cleaved by RNase H and exonuclease. Alternatively, the isolated RNA molecule produced by RNase H cleavage can be combined with exonuclease in a suitable buffer and reacted under conditions to provide the ASncmtRNA sequentially cleaved by RNase H and exonuclease. The exonuclease can be 3′ exonuclease or 5′ exonuclease, depending on the oligo used for the RNase cleavage process. For example, in FIG. 7A for oligonucleotide 1537, a 5′ exonuclease is used. The exonuclease cleavage results in a double stranded region that is recognized and cleaved by Dicer. 5′ or 3′ exonucleases may be employed (e.g. Exonuclease I, New England BioLabs catalog # M0293S or M0293L) and can be used according to the supplier's direction, or modifications thereof known to one skilled in the art. The resulting RNA molecule can be isolated and used in the methods as described herein. It is also possible to prepare the desired RNA synthetically by methods known to one skilled in the art.

E. Methods for Producing RNA from ASncmtRNA Sequentially Cleaved by RNase H, Exonuclease, and Dicer.

The sequentially RNase H and exonuclease cleaved RNA can be further processed without cells, in vitro, by the reaction described in FIGS. 7A and 7B. The reaction solution from the exonuclease cleavage can be suitably adjusted and a suitable Dicer can be added, and then reacted under suitable conditions to produce the ASncmtRNA sequentially cleaved by RNase H, exonuclease, and Dicer. Alternatively, the isolated RNA produced by sequential RNase H and exonuclease cleavage can be combined with Dicer in a suitable buffer and reacted under conditions to provide the ASncmtRNA sequentially cleaved by sequentially cleaved by RNase H, exonuclease, and Dicer, also referred to as Mito-miR or Mito-miRNA. Dicer is readily available (e.g. Genlantis Dicer Enzyme Kit catalog #T510002) and can be used according to the supplier's direction, or modifications thereof known to one skilled in the art. The Mito-miRNA produced by Dicer is double stranded, each strand being 15 to 30 nucleotides, 17 to 27 nucleotides, 19 to 25 nucleotides, or approximately 22 nuceotides long, and can be isolated as a duplex molecule or the strands separated and isolated as single strands. The duplex has a 3′ overhang region, i.e. a single stranded region at the 3′ end of each strand of 1 to 5 or 2 to 3 nucleotides (Park et al., Nature, 475:201-205, 2011, the disclosure of which is hereby incorporated by reference herein in its entirety). The resulting RNA molecules can be isolated and used in the methods as described herein, or can be prepared synthetically. It is also possible to prepare the desired RNA molecules synthetically by methods known to one skilled in the art. For example, RNA comprising a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO: 119, SEQ ID NO: 120, and SEQ ID NO: 121; or consisting of a sequence selected from the group consisting of SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO: 120, and SEQ ID NO: 121 can be made by synthetic methods well known to those skilled in the art. In some embodiments, the desired RNA is single stranded or double stranded, and single stranded RNA or one or both strands of double stranded RNA can be synthesized with suitable modifications as discussed herein. In some embodiments, the analogous DNA can be synthesized, to provide single stranded or double stranded DNA molecules analogous to the Mito-miRNA molecules.

In one aspect, provided herein is an isolated set of more than one RNA molecules, wherein said isolated set of more than one RNA molecules is prepared by the method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide a non-coding chimeric mitochondrial RNA molecule sequentially cleaved by RNase H and exonuclease; (c) digesting the non-coding chimeric mitochondrial RNA molecule sequentially cleaved by RNase H and exonuclease with Dicer to provide a non-coding chimeric mitochondrial RNA molecule sequentially cleaved by RNase H, exonuclease and Dicer; and isolating the set of RNA molecules resulting from the non-coding chimeric mitochondrial RNA molecule sequentially cleaved by RNase H, exonuclease and Dicer, to provide the isolated set of more than one RNA molecules.

In one aspect, provided herein is an isolated set of more than one RNA molecules, wherein said isolated set of more than one RNA molecules comprises more than one sequence analogous to a sequence resulting from a non-coding chimeric mitochondrial RNA molecule sequentially cleaved by RNase H, exonuclease and Dicer, wherein the non-coding chimeric mitochondrial RNA molecule sequentially cleaved by RNase H, exonuclease and Dicer is prepared by a method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide a non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the non-coding chimeric mitochondrial RNA molecule sequentially cleaved by RNase H, exonuclease and Dicer. In some embodiments, the isolated set of more than one RNA molecules consists essentially of more than one sequence analogous to the RNA molecule resulting from the non-coding chimeric mitochondrial RNA molecule sequentially cleaved by RNase H, exonuclease and Dicer. In some embodiments, the isolated set of more than one RNA molecules consists of more than one sequence analogous to the RNA molecule resulting from the non-coding chimeric mitochondrial RNA molecule sequentially cleaved by RNase H, exonuclease and Dicer. In some embodiments, the isolated set of more than one RNA molecules comprises more than one sequence identical to the RNA molecule resulting from the non-coding chimeric mitochondrial RNA molecule sequentially cleaved by RNase H, exonuclease and Dicer. In some embodiments, the isolated set of more than one RNA molecules consists essentially of more than one sequence identical to the RNA molecule resulting from the non-coding chimeric mitochondrial RNA molecule sequentially cleaved by RNase H, exonuclease and Dicer. In some embodiments, the isolated set of more than one RNA molecules consists of more than one sequence identical to the RNA molecule resulting from the non-coding chimeric mitochondrial RNA molecule sequentially cleaved by RNase H, exonuclease and Dicer.

In one aspect, provided herein is an isolated set of more than one RNA molecules, wherein said isolated set of more than one RNA molecules comprises all sequences analogous to a set of more than one RNA molecules resulting from a non-coding chimeric mitochondrial RNA molecule sequentially cleaved by RNase H, exonuclease and Dicer, wherein the non-coding chimeric mitochondrial RNA molecule sequentially cleaved by RNase H, exonuclease and Dicer is prepared by a method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide a non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the set of RNA molecules resulting from the non-coding chimeric mitochondrial RNA molecule sequentially cleaved by RNase H, exonuclease and Dicer. In some embodiments, the isolated set of more than one RNA molecules consists essentially of all sequences analogous to the set of more than one RNA molecules resulting from the non-coding chimeric mitochondrial RNA molecule sequentially cleaved by RNase H, exonuclease and Dicer. In some embodiments, the isolated set of more than one RNA molecules consists of all sequences analogous to the set of more than one RNA molecules resulting from a non-coding chimeric mitochondrial RNA molecule sequentially cleaved by RNase H, exonuclease and Dicer. In some embodiments, the isolated set of more than one RNA molecules comprises all sequences identical to the set of more than one RNA molecules resulting from a non-coding chimeric mitochondrial RNA molecule sequentially cleaved by RNase H, exonuclease and Dicer. In some embodiments, the isolated set of more than one RNA molecules consists essentially of all sequences identical to the set of more than one RNA molecules resulting from the non-coding chimeric mitochondrial RNA molecule sequentially cleaved by RNase H, exonuclease and Dicer. In some embodiments, the isolated set of more than one RNA molecules consists of all sequences identical to the set of more than one RNA molecules resulting from a non-coding chimeric mitochondrial RNA molecule sequentially cleaved by RNase H, exonuclease and Dicer.

In some embodiments, two strands sufficiently complementary to form double stranded RNA can be readily synthesized and can be annealed to provide a double stranded RNA molecule. In some embodiments, a strand comprising the sequence of SEQ ID NO:7 and a strand comprising the sequence of SEQ ID NO:96 can be synthesized and annealed to form the isolated double stranded RNA molecule. In some embodiments, a strand comprising the sequence of SEQ ID NO:8 and a strand comprising the sequence of SEQ ID NO:97 can be synthesized and annealed to form the isolated double stranded RNA molecule. In some embodiments, a strand comprising the sequence of SEQ ID NO:9 and a strand comprising the sequence of SEQ ID NO:98 can be synthesized and annealed to form the isolated double stranded RNA molecule. In some embodiments, a strand comprising the sequence of SEQ ID NO: 10 and a strand comprising the sequence of SEQ ID NO:99 can be synthesized and annealed to form the isolated double stranded RNA molecule. In some embodiments, a strand comprising the sequence of SEQ ID NO: 11 and a strand comprising the sequence of SEQ ID NO: 100 can be synthesized and annealed to form the isolated double stranded RNA molecule. In some embodiments, a strand comprising the sequence of SEQ ID NO: 12 and a strand comprising the sequence of SEQ ID NO: 101 can be synthesized and annealed to form the isolated double stranded RNA molecule. In some embodiments, a strand comprising the sequence of SEQ ID NO: 13 and a strand comprising the sequence of SEQ ID NO: 102 can be synthesized and annealed to form the isolated double stranded RNA molecule. In some embodiments, a strand comprising the sequence of SEQ ID NO: 14 and a strand comprising the sequence of SEQ ID NO: 103 can be synthesized and annealed to form the isolated double stranded RNA molecule. In some embodiments, a strand comprising the sequence of SEQ ID NO: 15 and a strand comprising the sequence of SEQ ID NO: 104 can be synthesized and annealed to form the isolated double stranded RNA molecule. In some embodiments, a strand comprising the sequence of SEQ ID NO: 16 and a strand comprising the sequence of SEQ ID NO: 105 can be synthesized and annealed to form the isolated double stranded RNA molecule. In some embodiments, a strand comprising the sequence of SEQ ID NO: 17 and a strand comprising the sequence of SEQ ID NO: 106 can be synthesized and annealed to form the isolated double stranded RNA molecule. In some embodiments, a strand comprising the sequence of SEQ ID NO: 18 and a strand comprising the sequence of SEQ ID NO: 107 can be synthesized and annealed to form the isolated double stranded RNA molecule. In some embodiments, a strand comprising the sequence of SEQ ID NO: 19 and a strand comprising the sequence of SEQ ID NO: 108 can be synthesized and annealed to form the isolated double stranded RNA molecule.

In some embodiments, the isolated RNA molecule prepared from an ASncmtRNA molecule sequentially cleaved by RNase H, exonuclease, and Dicer, or prepared synthetically, corresponds to and/or comprises a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO: 121. In some embodiments, the isolated RNA molecule is double stranded RNA, and one strand of the double stranded RNA molecule corresponds to and/or comprises a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121. In some embodiments, the isolated RNA molecule is double stranded RNA, and one strand of the double stranded RNA molecule corresponds to and/or consists essentially of the sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. In some embodiments, the isolated RNA molecule is double stranded RNA, and one strand of the double stranded RNA molecule corresponds to and/or consists of a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

In some embodiments of any of the isolated RNA molecules, including pharmaceutical compositions comprising the isolated RNA molecules, methods of making the isolated RNA molecules and methods of using the isolated RNA molecules, including use in kits, articles of manufacture, manufacture of medicaments, use for treatment of a cancer, and methods of treating a cancer or causing apoptosis in a tumor cell, as described herein, the isolated RNA molecule can be a single stranded or a double stranded RNA molecule. In some embodiments, the isolated RNA molecule is an isolated single stranded RNA molecule. In some embodiments, the isolated RNA molecule is an isolated double stranded RNA molecule. In some embodiments, the isolated RNA molecule corresponds to and/or comprises a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121. In some embodiments the isolated RNA molecule is an isolated double stranded RNA molecule corresponding to and/or comprising a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121. In some embodiments, the isolated RNA molecule is an isolated double stranded RNA molecule, wherein one strand of the double stranded RNA molecule corresponds to and/or consists of a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

a. Oligonucleotide Modifications

The naturally occurring internucleoside linkage of RNA and DNA is a 3′ to 5 phosphodiester linkage. The oligonucleotides, for example, the antisense oligonucleotides used to anneal with ASncmtRNA for subsequent RNase H treatment, or the RNA molecules as described herein, such as the RNA molecules resulting from ASncmtRNA cleaved by RNase H, ASncmtRNA sequentially cleaved by RNase H and exonuclease, or ASncmtRNA sequentially cleaved by RNase H, exonuclease and Dicer, including the RNA molecules described herein having the analogous sequences to RNA resulting from ASncmtRNA cleaved by RNase H, ASncmtRNA sequentially cleaved by RNase H and exonuclease, or ASncmtRNA sequentially cleaved by RNase H, exonuclease and Dicer, can have one or more modified, i.e. non-naturally occurring, internucleoside linkages. With respect to therapeutics, modified internucleoside linkages are often selected over oligonucleotides having naturally occurring internucleoside linkages because of desirable properties such as, for example, enhanced cellular uptake, enhanced affinity for target nucleic acids, and increased stability in the presence of nucleases.

Oligonucleotides, such as an antisense oligonucleotides and RNA molecules or analogous DNA molecules as described herein having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In one embodiment, oligonucleotides targeted to the ASncmtRNA molecules described herein or the RNA molecules or analogous DNA molecules as described herein comprise one or more modified internucleoside linkages. In some embodiments, the modified internucleoside linkages are phosphorothioate linkages. In other embodiments, each internucleoside linkage of an oligonucleotide compound is a phosphorothioate internucleoside linkage.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific though nonlimiting examples of oligonucleotides, including RNA molecules or analogous DNA molecules as described herein, useful in the methods as described herein include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In some embodiments, modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thiono-phosphoramidates, thionoalkylphosphonates, thionoalkylphospho-triesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof) can also be employed. Various salts, mixed salts and free acid forms are also included. Oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other embodiments, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is hereby incorporated by reference herein in their entirety. Further teaching of PNA compounds can be found in Nielsen et al., Science, 1991, 254, 1497-1500.

Representative United States patents that teach the preparation of the above phosphorus-containing and non-phosphorus-containing linkages include, but are not limited to, U.S. Pat. Nos. 5,541,306; 5,550,111; 5,563,253; 5,571,799; 5,587,361; 5,194,599; 5,565,555; 5,527,899; 5,721,218; 5,672,697 and 5,625,050, 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is hereby incorporated by reference herein in their entirety.

Modified oligonucleotides, such as an antisense oligonucleotide and RNA molecules or analogous DNA molecules as described herein may also contain one or more substituted sugar moieties. For example, the furanosyl sugar ring can be modified in a number of ways including substitution with a substituent group, bridging to form a bicyclic nucleic acid “BNA” and substitution of the 4′-O with a heteroatom such as S or N(R) as described in U.S. Pat. No. 7,399,845, hereby incorporated by reference herein in its entirety. Other examples of BNAs are described in published International Patent Application No. WO 2007/146511, hereby incorporated by reference herein in its entirety.

The oligonucleotides, such as an antisense oligonucleotide and RNA molecules or analogous DNA molecules as described herein can optionally contain one or more nucleotides having modified sugar moieties. Sugar modifications may impart nuclease stability, binding affinity or some other beneficial biological property to the antisense compounds and RNA molecules or analogous DNA molecules as described herein. The furanosyl sugar ring of a nucleoside can be modified in a number of ways including, but not limited to: addition of a substituent group, particularly at the 2′ position; bridging of two non-geminal ring atoms to form a bicyclic nucleic acid (BNA); and substitution of an atom or group such as —S—, —N(R)— or —C(R1)(R2) for the ring oxygen at the 4′-position. Modified sugars include, but are not limited to: substituted sugars, especially 2′-substituted sugars having a 2′-F, 2′-OCH₂ (2′-OMe) or a 2′-O(CH₂)₂—OCH₃ (2′-O-methoxyethyl or 2′-MOE) substituent group; and bicyclic modified sugars (BNAs), having a 4′-(CH₂)n-O-2′ bridge, where n=1 or n=2. Methods for the preparations of modified sugars are well known to those skilled in the art.

In certain embodiments, a 2′-modified nucleoside has a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In certain such embodiments, the bicyclic sugar moiety is an L sugar in the beta configuration.

In other embodiments, the bicyclic sugar moiety comprises a bridge group between the 2′ and the 4′-carbon atoms. In certain such embodiments, the bridge group comprises from 1 to linked biradical groups. In certain embodiments, the bicyclic sugar moiety comprises from 1 to 4 linked biradical groups. In certain embodiments, the bicyclic sugar moiety comprises 2 or 3 linked biradical groups. In certain embodiments, the bicyclic sugar moiety comprises 2 linked biradical groups. In certain embodiments, a linked biradical group is selected from —O—, —S—, —N(R1)-, —C(R1)(R2)-, —C(R1)=C(R1)-, C(R1)=N—, —C(═NR1)-, —Si(R1)(R2)-, —S(═O)2-, —S(═O)—, —C(═O)— and —C(═S)—; where each R1 and R2 is, independently, H, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, a heterocycle radical, a substituted hetero-cycle radical, heteroaryl, substituted heteroaryl, C5-C7 alicyclic radical, substituted C5-C7 alicyclic radical, halogen, substituted oxy (—O—), amino, substituted amino, azido, carboxyl, substituted carboxyl, acyl, substituted acyl, CN, thiol, substituted thiol, sulfonyl (S(═O)2-H), substituted sulfonyl, sulfoxyl (S(═O)—H) or substituted sulfoxyl; and each substituent group is, independently, halogen, C1-C12 alkyl, substituted C1-C12 alkyl, C2-C12 alkenyl, substituted C2-C12 alkenyl, C2-C12 alkynyl, substituted C2-C12 alkynyl, amino, substituted amino, acyl, substituted acyl, C1-C12 aminoalkyl, C1-C12 aminoalkoxy, substituted C1-C12 aminoalkyl, substituted C1-C12 aminoalkoxy or a protecting group.

Oligonucleotides, such as an antisense oligonucleotide and RNA molecules or analogous DNA molecules as described herein may also include nucleobase (often referred to in the art simply as “base”) modifications or substitutions. Nucleobase modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to oligonucleotide compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an oligonucleotide compound (such as an antisense oligonucleotide compound) for a target nucleic acid (such as an ASncmtRNA).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds and RNA molecules or analogous DNA molecules as described herein include 5-substituted pyrimidines, 6-azapyrimidines and N-2, N-6 and O-6 substituted purines, including 2 aminopropyladenine, 5-propynyluracil and 5-propynylcytosine.

As used herein, “unmodified” or “natural” nucleobases include the purine bases adenine (A) and guanine (G), and the pyrimidine bases thymine (T), cytosine (C) and uracil (U).

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), O-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993.

Representative United States patents that teach the preparation of certain of the above noted modified nucleobases as well as other modified nucleobases include, but are not limited to, U.S. Pat. Nos. 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, each of which is hereby incorporated by reference herein in their entirety.

### IV. Methods of Using Isolated RNA

A. Methods for Treating Cancer, Pre-Cancer, or Elimination of Cancer Stem Cells

Provided herein is a method of treating a cancer, pre-cancer, or method of eliminating a cancer stem cell in a subject comprising administering to the subject in need thereof a therapeutically effective amount of the isolated RNA molecule or pharmaceutical composition comprising the isolated RNA molecule as described herein.

In some embodiments of the method of treating a cancer or pre-cancer, or method of eliminating a cancer stem cell in a subject, the method comprises administering to the subject in need thereof a therapeutically effective amount of the isolated RNA molecule, or pharmaceutical composition comprising the isolated RNA molecule, wherein the RNA molecule is prepared by a method comprising (a) annealing one or more oligonucleotides with a non-coding chimeric mitochondrial RNA molecule and digesting the RNA:oligonucleotide hybrid with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) optionally digesting the non-coding chimeric mitochondrial RNA molecule cleaved by RNase H with an exonuclease to provide a non-coding chimeric mitochondrial RNA molecule sequentially cleaved by RNase H and exonuclease; (c) optionally digesting the non-coding chimeric mitochondrial RNA molecule sequentially cleaved by RNase H and exonuclease with Dicer to provide a non-coding chimeric mitochondrial RNA molecule sequentially cleaved by RNase H, exonuclease and Dicer; and isolating the non-coding chimeric mitochondrial RNA molecule cleaved by RNase H, and/or optionally isolating the non-coding chimeric mitochondrial RNA molecule sequentially cleaved by RNase H and exonuclease, and/or optionally isolating the non-coding chimeric mitochondrial RNA molecule sequentially cleaved by RNase H, exonuclease and Dicer, to provide the isolated RNA molecule. In some embodiments, the method of treatment results in apoptosis of at least one cancer cell, pre-cancer cell, or cancer stem cell.

In some embodiments of the method of treating a cancer or pre-cancer, or method of eliminating a cancer stem cell in a subject, the method comprises administering to the subject in need thereof a therapeutically effective amount of the isolated RNA molecule, or pharmaceutical composition comprising the isolated RNA molecule, wherein said isolated RNA molecule comprises a sequence analogous to an RNA molecule selected from the group consisting of a non-coding chimeric mitochondrial RNA molecule cleaved by RNase H; a non-coding chimeric mitochondrial RNA molecule sequentially cleaved by RNase H and exonuclease; and a non-coding chimeric mitochondrial RNA molecule sequentially cleaved by RNase H, exonuclease and Dicer; wherein the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer are prepared by a method comprising (a) annealing one or more oligonucleotides with a non-coding chimeric mitochondrial RNA molecule and digesting with an RNase H to provide the non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide the non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the non-coding chimeric mitochondrial RNA molecule sequentially cleaved by RNase H, exonuclease and Dicer. In some embodiments, said isolated RNA molecule consists essentially of a sequence analogous to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer. In some embodiments, said isolated RNA molecule consists of a sequence/corresponds to a RNA sequence analogous to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer. In some embodiments, said isolated RNA molecule comprises a sequence identical to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer. In some embodiments, said isolated RNA molecule consists essentially of a sequence identical to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer. In some embodiments, said isolated RNA molecule consists of a sequence identical to an RNA molecule selected from the group consisting of the non-coding chimeric mitochondrial RNA molecule cleaved by RNase H; the non-coding chimeric mitochondrial RNA molecule sequentially cleaved by RNase H and exonuclease; and the non-coding chimeric mitochondrial RNA molecule sequentially cleaved by RNase H, exonuclease and Dicer. In some embodiments, the method of treatment results in apoptosis of at least one cancer cell, pre-cancer cell, or cancer stem cell.

In some embodiments of the method of treating a cancer or pre-cancer, or method of eliminating a cancer stem cell in a subject, the method comprises administering to the subject in need thereof a therapeutically effective amount of an isolated set of more than one RNA molecules, or pharmaceutical composition comprising the isolated set of more than one RNA molecules, wherein said isolated set of more than one RNA molecules is prepared by the method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide a non-coding chimeric mitochondrial RNA molecule sequentially cleaved by RNase H and exonuclease; (c) digesting the non-coding chimeric mitochondrial RNA molecule sequentially cleaved by RNase H and exonuclease with Dicer to provide a non-coding chimeric mitochondrial RNA molecule sequentially cleaved by RNase H, exonuclease and Dicer; and isolating the set of RNA molecules resulting from the non-coding chimeric mitochondrial RNA molecule sequentially cleaved by RNase H, exonuclease and Dicer, to provide the isolated set of more than one RNA molecules. In some embodiments, the method of treatment results in apoptosis of at least one cancer cell, pre-cancer cell, or cancer stem cell.

In some embodiments of the method of treating a cancer or pre-cancer, or method of eliminating a cancer stem cell in a subject, the method comprises administering to the subject in need thereof a therapeutically effective amount of an isolated set of more than one RNA molecules, or pharmaceutical composition comprising the isolated set of more than one RNA molecules, wherein said isolated set of more than one RNA molecules comprises more than one sequence analogous a sequence resulting from a non-coding chimeric mitochondrial RNA molecule sequentially cleaved by RNase H, exonuclease and Dicer, wherein the non-coding chimeric mitochondrial RNA molecule sequentially cleaved by RNase H, exonuclease and Dicer is prepared by a method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide a non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the non-coding chimeric mitochondrial RNA molecule sequentially cleaved by RNase H, exonuclease and Dicer. In some embodiments, the isolated set of more than one RNA molecules consists essentially of more than one sequence analogous to the RNA molecule resulting from the non-coding chimeric mitochondrial RNA molecule sequentially cleaved by RNase H, exonuclease and Dicer. In some embodiments, the isolated set of more than one RNA molecules consists of more than one sequence analogous to the RNA molecule resulting from the non-coding chimeric mitochondrial RNA molecule sequentially cleaved by RNase H, exonuclease and Dicer. In some embodiments, the isolated set of more than one RNA molecules comprises more than one sequence identical to the RNA molecule resulting from the non-coding chimeric mitochondrial RNA molecule sequentially cleaved by RNase H, exonuclease and Dicer. In some embodiments, the isolated set of more than one RNA molecules consists essentially of more than one sequence identical to the RNA molecule resulting from the non-coding chimeric mitochondrial RNA molecule sequentially cleaved by RNase H, exonuclease and Dicer. In some embodiments, the isolated set of more than one RNA molecules consists of more than one sequence identical to the RNA molecule resulting from the non-coding chimeric mitochondrial RNA molecule sequentially cleaved by RNase H, exonuclease and Dicer. In some embodiments, the method of treatment results in apoptosis of at least one cancer cell, pre-cancer cell or cancer stem cell.

In some embodiments of the method of treating a cancer or pre-cancer, or method of eliminating a cancer stem cell in a subject, the method comprises administering to the subject in need thereof a therapeutically effective amount of an isolated set of more than one RNA molecules, or pharmaceutical composition comprising the isolated set of more than one RNA molecules, wherein said isolated set of more than one RNA molecules comprises all sequences analogous to a set of more than one RNA molecules resulting from a non-coding chimeric mitochondrial RNA molecule sequentially cleaved by RNase H, exonuclease and Dicer, wherein the non-coding chimeric mitochondrial RNA molecule sequentially cleaved by RNase H, exonuclease and Dicer is prepared by a method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide a non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the set of RNA molecules resulting from the non-coding chimeric mitochondrial RNA molecule sequentially cleaved by RNase H, exonuclease and Dicer. In some embodiments, the isolated set of more than one RNA molecules consists essentially of all sequences analogous to the set of more than one RNA molecules resulting from a non-coding chimeric mitochondrial RNA molecule sequentially cleaved by RNase H, exonuclease and Dicer. In some embodiments, the isolated set of more than one RNA molecules consists of all sequences analogous to the set of more than one RNA molecules resulting from a non-coding chimeric mitochondrial RNA molecule sequentially cleaved by RNase H, exonuclease and Dicer. In some embodiments, the isolated set of more than one RNA molecules consists essentially of all sequences analogous to the set of more than one RNA molecules resulting from the non-coding chimeric mitochondrial RNA molecule sequentially cleaved by RNase H, exonuclease and Dicer. In some embodiments, the isolated set of more than one RNA molecules consists of all sequences analogous to the set of more than one RNA molecules resulting from a non-coding chimeric mitochondrial RNA molecule sequentially cleaved by RNase H, exonuclease and Dicer. In some embodiments, the isolated set of more than one RNA molecules comprises all sequences identical to the set of more than one RNA molecules resulting from a non-coding chimeric mitochondrial RNA molecule sequentially cleaved by RNase H, exonuclease and Dicer. In some embodiments, the isolated set of more than one RNA molecules consists essentially of all sequences identical to the set of more than one RNA molecules resulting from the non-coding chimeric mitochondrial RNA molecule sequentially cleaved by RNase H, exonuclease and Dicer. In some embodiments, the isolated set of more than one RNA molecules consists of all sequences identical to the set of more than one RNA molecules resulting from a non-coding chimeric mitochondrial RNA molecule sequentially cleaved by RNase H, exonuclease and Dicer. In some embodiments, the method of treatment results in apoptosis of at least one cancer cell, pre-cancer cell or cancer stem cell.

In some embodiments of the method of treating a cancer or pre-cancer, or method of eliminating a cancer stem cell in a subject, the method comprises administering to the subject in need thereof a therapeutically effective amount of the isolated RNA molecule, or pharmaceutical composition comprising the isolated RNA molecule, wherein the RNA molecule comprises a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121. In some embodiments of the method of treating a cancer or pre-cancer, or method of eliminating a cancer stem cell in a subject, the isolated RNA molecule is an isolated double stranded RNA molecule comprising a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121. In some embodiments of the method of treating a cancer or pre-cancer, or method of eliminating a cancer stem cell in a subject, the isolated RNA molecule is an isolated double stranded RNA molecule, wherein one strand of the double stranded RNA molecule consists of a sequence/corresponds to a RNA sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO: 19. In some embodiments, the method of treatment results in apoptosis of at least one cancer cell, pre-cancer cell, or cancer stem cell.

In some embodiments of the method of treating a cancer in a subject, the cancer is a solid tumor or a hematological cancer (i.e. non-solid cancer). In some embodiments, the cancer is non-solid cancer selected from the group consisting of multiple myeloma, acute myeloid leukemia, acute lymphoblastic leukemia, chronic myelogenous leukemia, chronic lymphocytic leukemia, acute nonlymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, undifferentiated cell leukemia, idiopathic myelofibrosis, lymphoma (such as Non-Hodgkin's lymphoma, and Hodgkin's lymphoma), and myelodysplastic syndrome; or a solid tumor selected from the group consisting of squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, brain cancer, cervical cancer, ovarian cancer, liver cancer, sarcoma, bladder cancer, hepatoma, breast cancer, colon cancer, colorectal cancer, endometrial or uterine carcinoma, oralpharyngeal cancer, salivary gland carcinoma, renal cancer, liver cancer, prostate cancer, vulval cancer, thyroid cancer, hepatic carcinoma, gastric cancer, melanoma, and various types of head and neck cancer.

The methods of treating cancer as described herein is also directed to methods for inhibiting the symptoms or conditions (disabilities, impairments) associated with cancer (e.g., metastatic cancer or relapsed cancer). As such, it is not required that all effects of the condition be entirely prevented or reversed, although the effects of the presently disclosed methods likely extend to a significant therapeutic benefit for the individual. As such, a therapeutic benefit is not necessarily a complete prevention or cure for the condition, but rather, can encompass a result which includes reducing or preventing the symptoms that result from cancer (e.g., metastatic cancer or relapsed cancer), reducing or preventing the occurrence of such symptoms (either quantitatively or qualitatively), reducing the severity of such symptoms or physiological effects thereof, and/or enhancing the recovery of the individual after experiencing cancer (e.g., metastatic cancer or relapsed cancer) symptoms.

The methods provided herein involve isolated RNA molecules effective in treating a variety of cancers or pre-cancers, or eliminating cancer stem cells. These isolated RNA molecules are downstream elements formed by the treatment of cancer with oligonucleotides complementary to an antisense non-coding chimeric mitochondrial RNA (ASncmtRNA) molecule as described herein or in U.S. Pat. No. 8,318,686. The RNA formed during this process, as outlined in FIGS. 7A and 7B, provide useful cancer therapeutics further downstream in this process, and can be readily isolated or prepared synthetically for use as a cancer therapeutic. As these downstream elements result in fewer processing steps in vivo, the methods described herein provide more precise and selective therapeutics for treating essentially any cancer or pre-cancer or eliminating cancer stem cells.

In some embodiments of the method of treating a cancer or pre-cancer, or method of eliminating a cancer stem cell in a subject comprising administering to the subject in need thereof a therapeutically effective amount of an isolated RNA molecule prepared from an ASncmtRNA molecule sequentially cleaved by RNase H, exonuclease, and Dicer. In some embodiments, the method comprises administering to the subject in need thereof a therapeutically effective amount of an isolated synthetic RNA molecule having a sequence analogous to the isolated RNA molecule prepared from an ASncmtRNA molecule sequentially cleaved by RNase H, exonuclease and Dicer. In some embodiments, the isolated RNA molecule prepared from an ASncmtRNA molecule sequentially cleaved by RNase H, exonuclease, and Dicer comprises the sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121. In some embodiments, the isolated RNA molecule prepared from an ASncmtRNA molecule sequentially cleaved by RNase H, exonuclease, and Dicer is a double stranded RNA molecule, wherein one strand of the double stranded RNA molecule comprises the sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, and the other strand comprises the sequence selected from the group consisting of SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO: 107, and SEQ ID NO: 108. In some embodiments, the isolated RNA molecule prepared from an ASncmtRNA molecule sequentially cleaved by RNase H, exonuclease, and Dicer is a double stranded RNA molecule, wherein one strand of the double stranded RNA molecule consists essentially of the sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO: 19. In some embodiments, the isolated RNA molecule prepared from an ASncmtRNA molecule sequentially cleaved by RNase H, exonuclease, and Dicer is a double stranded RNA molecule, wherein one strand of the double stranded RNA molecule consists of the sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. In some embodiments, the method of treatment results in apoptosis of at least one cancer cell, pre-cancer cell, or cancer stem cell.

In some embodiments of the method of treating a cancer or pre-cancer, or method of eliminating a cancer stem cell in a subject comprising administering to the subject in need thereof a therapeutically effective amount of an isolated RNA molecule prepared from an ASncmtRNA molecule sequentially cleaved by RNase H, exonuclease, and Dicer the isolated RNA is double stranded RNA, wherein one strand comprises the sequence of SEQ ID NO:7 and the complementary strand comprises the sequence of SEQ ID NO:96, one strand comprises the sequence of SEQ ID NO:8 and the complementary strand comprises the sequence of SEQ ID NO:97, one strand comprises the sequence of SEQ ID NO:9 and the complementary strand comprises the sequence of SEQ ID NO:98, one strand comprises the sequence of SEQ ID NO: 10 and the complementary strand comprises the sequence of SEQ ID NO:99, one strand comprises the sequence of SEQ ID NO: 11 and the complementary strand comprises the sequence of SEQ ID NO: 100, one strand comprises the sequence of SEQ ID NO: 12 and the complementary strand comprises the sequence of SEQ ID NO: 101, one strand comprises the sequence of SEQ ID NO: 13 and the complementary strand comprises the sequence of SEQ ID NO: 102, one strand comprises the sequence of SEQ ID NO: 14 and the complementary strand comprises the sequence of SEQ ID NO: 103, one strand comprises the sequence of SEQ ID NO: 15 and the complementary strand comprises the sequence of SEQ ID NO: 104, one strand comprises the sequence of SEQ ID NO: 16 and the complementary strand comprises the sequence of SEQ ID NO: 105, one strand comprises the sequence of SEQ ID NO: 17 and the complementary strand comprises the sequence of SEQ ID NO: 106, one strand comprises the sequence of SEQ ID NO: 18 and the complementary strand comprises the sequence of SEQ ID NO: 107, one strand comprises the sequence of SEQ ID NO: 19 and the complementary strand comprises the sequence of SEQ ID NO: 108. In some embodiments, the method of treatment results in apoptosis of at least one cancer cell, pre-cancer cell, or cancer stem cell.

As non-limiting examples, treatment according to the present invention may be provided as a daily dosage of an isolated RNA molecule in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

In any of the embodiments of the methods of treating a cancer or pre-cancer, or method of eliminating a cancer stem cell in a subject, an analogous synthetic RNA molecule or analogous synthetic DNA molecule, or a pharmaceutical composition thereof can be administered in a therapeutically effective amount to a subject in need thereof in these methods, i.e. as described above for the isolated RNA molecule or pharmaceutical composition thereof.

B. Down Regulation of Proteins Involved in Apoptosis by Mito-miRs

Provided herein is a method of causing apoptosis in a tumor cell comprising contacting the tumor cell with one or more isolated RNA molecules or pharmaceutical composition comprising the one or more isolated RNA molecules as described herein.

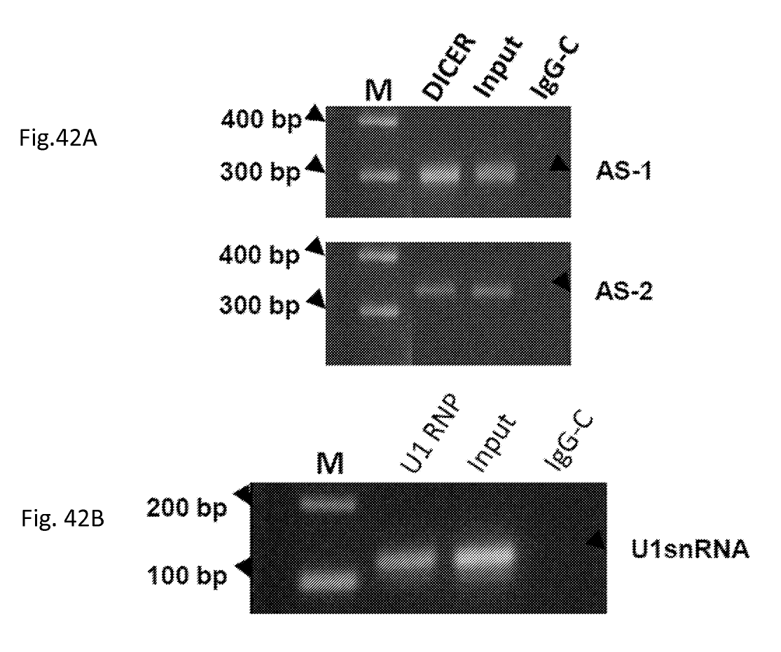

In another aspect, the Mito-miRs prepared as discussed herein are useful for interfering with the expression of a number of proteins involved in the regulation of cancer cells, including proteins involved in one or more of apoptosis, the regulation of cell growth, cell migration, cell invasion and metastasis. In one embodiment, the protein is selected from the group consisting of survivin, cyclin D1, cyclin B1, FKBP38, N-cadherin and caveolin. Without being limited to mechanism, the Mito-miRs described herein can hybridize to untranslated regions (UTR) of mRNA from a variety of such proteins. The binding to the UTR inhibits the expression of the protein, resulting in a reduction in the protein in cells. This down regulation of these proteins involved in the regulation of cancer cells, including proteins involved in one or more of apoptosis, the regulation of cell growth, cell migration, cell invasion and metastasis, results in cancer cell death. As such, the treatment of cancer as described herein results from treatment with one or more Mito-miRs as described herein, or with an isolated RNA molecule as described herein that reacts in the cell to form one or more Mito-miRs as described herein, wherein the Mito-miR results in the down regulation of expression of one or more proteins, wherein the reduced expression of the protein in a tumor cell results in cell death. In one embodiment, the expression of one or more proteins selected from the group consisting of survivin, cyclin D1, cyclin B1, FKBP38, N-cadherin and caveolin is reduced by treatment with Mito-miRs or other isolated RNA molecules as described herein. Mito-miRs as described herein that can bind to UTR of, for example, survivin, cyclin D1 and cyclin B1 are shown in FIGS. 42A-C, respectively.

a. Down Regulation of Survivin

Survivin is a member of the Inhibitor of Apoptosis (IAP) family of proteins, and is up-regulated in virtually all human cancer cells. The IAP family of proteins plays an important cytoprotective function in cancer cells downstream of the intrinsic apoptosis pathway (Dohi et al., J. Biol. Chem., 2004, 279:34087-34090; Dohi et al., Mol Cell., 2007, 27:17-28; Altieri, D. C., Biochem. J., 2010, 30:199-205; Kang et al., J. Biol. Chem., 2011, 286:16758-16767). Down regulation of survivin will affect this anti-apoptotic function, and result in apoptosis of cancer cells. As described herein, the level of survivin in tumor cells is drastically reduced by treatment with antisense oligonucleotide to ASncmtRNA (See Examples 12, 14), and treatment with miRNA or other isolated RNA molecules as described herein will similarly reduce survivin levels and kill cancer cells. Treatment with the isolated RNA molecules as described herein result in the binding of Mito-miRs such as those shown in FIG. 42A, which can be complementary to a UTR of survivin mRNA. Such Mito-miRNA molecules are selective in down regulating survivin, and are ideal for treatment of a variety of cancers without effecting normal cells. Similarly activity has been seen with known miRs regulating the expression of survivin (Diakos et al., Blood, 2010, 16:4885-4893; Alajez et al., Cancer Res., 2011, 71:2381-2391).

b. Down Regulation of Cyclin D1

Cyclin D1 is involved in the regulation of cell growth, and is known to be required for the transition of cells from phase G1 to S (Vien Khach Lai, et al., Cell Cycle, 11:767-777, 2012; Jing Nie, et al., Carcinogenesis, 33:220-225, 2012; Qiong Jiang et al., BMC Cancer, 9:194-208, 2009). The down regulation of cyclin D1 results in an increase of cells in phase G1 and an increase in cell death. As described herein, the level of cyclin D1 in tumor cells is reduced by treatment with antisense oligonucleotide to ASncmtRNA (See Example 15), and treatment with miRNA or other isolated RNA molecules as described herein will similarly reduce cyclin D1 levels and kill cancer cells. Treatment with the isolated RNA molecules as described herein result in the binding of Mito-miRs such as those shown in FIG. 42B, which can be complementary to a UTR of cyclin D1 mRNA. Such Mito-miRNA molecules are selective in down regulating cyclin D1, and are ideal for treatment of a variety of cancers without effecting normal cells.

c. Down Regulation of Cyclin B1

Cyclin B1 is involved in the regulation of cell growth, and is known to induce mitosis (Debra J. Wolgemuth, Cell Cycle, 7:3509-3513, 2008; Vera Huang and Long-Cheng Li, RNA Biology, 9:269-273, 2012). The down regulation of cyclin B1 results in the inhibition of mitosis and an increase in cell death. As described herein, the level of cyclin B1 in tumor cells is reduced by treatment with antisense oligonucleotide to ASncmtRNA (See Example 15), and treatment with miRNA or other isolated RNA molecules as described herein will similarly reduce cyclin B1 levels and kill cancer cells. Treatment with the isolated RNA molecules as described herein result in the binding of Mito-miRs such as those shown in FIG. 42C, which can be complementary to a UTR of cyclin B1 mRNA. Such Mito-miRNA molecules are selective in down regulating cyclin B1, and are ideal for treatment of a variety of cancers without effecting normal cells.

d. Down Regulation of FKBP38

Bcl2 is a mitochondrial anti-apoptotic protein that is maintained in the mitochondria by the action of FKBP38 (Portier, B. P. and Taglialatela, G., J. Biol. Chem., 281:40493-40502, 2006; Shirane, M. and Nakayama, K. I., Nat. Cell Biol., 5:28-37, 2003; Wang, H. Q., et al., Human Mol. Gen., 14:1889-1902, 2005; Bai X, et al., Science, 318:977-980, 2007; Xuemin Wang, et al., J. Biol. Chem., 283:30482-30492, 2008). The down regulation of FKBP38 results in the migration of Bcl2 to the nucleus, where it becomes pro-apoptotic, resulting in death of cancer cells. As described herein, the level of FKBP38 in tumor cells is reduced by treatment with antisense oligonucleotide to ASncmtRNA, without affecting the level of Bcl2 (See Example 16), and treatment with miRNA or other isolated RNA molecules as described herein will similarly reduce FKBP38 levels and kill cancer cells. Treatment with the isolated RNA molecules as described herein result in the binding of Mito-miRs, which can be complementary to a UTR of FKBP38 mRNA. Such Mito-miRNA molecules are selective in down regulating FKBP38, and are ideal for treatment of a variety of cancers without effecting normal cells.

e. Down Regulation of N-Cadherin and Caveolin

N-cadherin and caveolin are involved in cell migration, cell invasion and metastasis (L. D. M. Derycke and M. E. Bracken, Int. J. Dev. Biol., 4:463-476, 2004; Lorena Lobos-Gonzalez et al., Pigment Cell Melanoma Res., 26:555-570, 2013; Fiucci, G., et al., Oncogene 21:2365-2375, 2002; Jean-Leon Maitre and Carl-Philipp Heisenberg, Current Biology 23:R626-R633,2013). The down regulation of N-cadherin and caveolin results in increased possibilities of apoptosis and death of cancer cells. As described herein, the level of N-cadherin and caveolin in tumor cells is reduced by treatment with antisense oligonucleotide to ASncmtRNA (See Example 15), and treatment with miRNA or other isolated RNA molecules as described herein will similarly reduce N-cadherin and caveolin levels and kill cancer cells. Treatment with the isolated RNA molecules as described herein result in the binding of Mito-miRs, which can be complementary to a UTR of N-cadherin or caveolin mRNA. Such Mito-miRNA molecules are selective in down regulating N-cadherin and caveolin, and are ideal for treatment of a variety of cancers without effecting normal cells.

### III. Methods of Treatment in Combination with Other Anticancer Therapies

In some aspects, any of the methods of treatment described herein can comprise administering one or more additional anticancer therapies to the subject. Various classes of anticancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anticancer agents that can be used. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are derived from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some aspects, the anticancer therapeutics can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, or etoposide.

In other embodiments, the anticancer therapeutics can be selected from bortezomib, cyclophosphamide, dexamethasone, doxorubicin, interferon-alpha, lenalidomide, melphalan, pegylated interferon-alpha, prednisone, thalidomide, or vincristine.

In other aspects, any of the methods of treatment described herein can include either autologous or allogenic stem cell transplantation therapy. Autologous stem cell transplantation is typically used for subjects under the age of 65 years who do not have substantial heart, lung, renal or liver dysfunction.

### IV. Pharmaceutical Compositions

In another aspect, provided herein is a pharmaceutical composition comprising one or more isolated RNA molecules, such as any of the isolated RNA molecules described herein. In some embodiments, provided herein is a pharmaceutical composition comprising one or more isolated RNA molecules comprising a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO: 120, and SEQ ID NO: 121. In some embodiments, the pharmaceutical composition comprises an isolated double stranded RNA molecule comprising a sequence selected from the group consisting of SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO: 120, and SEQ ID NO:121. In some embodiments, the pharmaceutical composition comprises an isolated double stranded RNA molecule, wherein one strand of the double stranded RNA molecule consists of a sequence/corresponds to a RNA sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

Also provided herein is a pharmaceutical composition comprising one or more isolated RNA molecules as described herein and a pharmaceutically acceptable vehicle or excipient.

The anticancer therapies, e.g. treatment by administering the isolated RNA molecules as disclosed herein, can be administered in the form of pharmaceutical compositions. These RNA molecules and pharmaceutical compositions thereof can be administered by a variety of routes including oral, rectal, cerebrospinal, transdermal, subcutaneous, topical, transmucosal, nasopharangeal, pulmonary, intravenous, intraperitonial, intramuscular, and intranasal. In some embodiments, the administration is a local administration. In some embodiments, the local administration is selected from the group consisting of administration into an organ, into a cavity, into a tissue, into a solid tumor, and subcutaneous administration. In some embodiments, the administration is systemic administration. In some embodiments, the systemic administration is intravenous or intraperitoneal administration. These compounds are effective as both injectable and oral compositions. Such compositions are prepared in a manner well known in the pharmaceutical art and comprise at least one active compound. When employed as oral compositions, the oligonucleotides and another disclosed herein are protected from acid digestion in the stomach by a pharmaceutically acceptable protectant.

Also provided herein are pharmaceutical compositions which contain, as the active ingredient, one or more of the isolated RNA molecules as disclosed herein associated with one or more pharmaceutically acceptable excipients or carriers. In making the compositions of this invention, the active ingredient is usually mixed with an excipient or carrier, diluted by an excipient or carrier or enclosed within such an excipient or carrier which can be in the form of a capsule, sachet, paper or other container. When the excipient or carrier serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active lyophilized compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients or carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In another aspect, one or more RNA molecules or DNA molecules as described herein are encapsulated within a microcarrier for deliver to an individual. In some embodiments the microcarrier encapsulates more than one RNA molecule and/or DNA molecule species. In some embodiments, the one or more one RNA molecule and/or DNA molecule species encapsulated within the microcarrier comprises a nucleic acid sequence selected from the group consisting of SEQ ID NOs:7-32. Methods of encapsulating oligonucleotides in microcarriers are well known in the art, and described, for example, in International application WO98/55495. Colloidal dispersion systems, such as microspheres, beads, macromolecular complexes, nanocapsules and lipid-based system, such as oil-in-water emulsions, micelles, mixed micelles and liposomes can provide effective encapsulation of oligonocelotides within microcarrier compositions. The encapsulation composition may further comprise any of a wide variety of components. These include, but are not limited to, alum, lipids, phospholipids, lipid membrane structures (LMS), polyethylene glycol (PEG) and other polymers, such as polypeptides, glycopeptides, and polysaccharides.

The compositions can be formulated in a unit dosage form, each dosage containing from about 5 mg to about 1000 mg or more, such as any of about 5 mg to about 900 mg, 5 mg to about 800 mg, about 5 mg to about 700 mg, about 5 mg to about 600 mg, about 5 mg to about 500 mg, about 10 mg to about 500 mg, about 15 mg to about 500 mg, about 20 mg to about 500 mg, about 25 mg to about 500 mg, about 30 mg to about 500 mg, about 35 mg to about 500 mg, about 40 mg to about 500 mg, about 45 mg to about 500 mg, or about 50 mg to about 500 mg, inclusive, including any range in between these values, of the active ingredient. The term “unit dosage forms” refers to physically discrete units suitable as unitary dosages for subjects, each unit containing a predetermined quantity of active material calculated to produce the desired therapeutic effect, in association with a suitable pharmaceutical excipient or carrier.

The anticancer therapies disclosed herein are effective over a wide dosage range and are generally administered in a therapeutically effective amount. It will be understood, however, that the amount of the anticancer therapies actually administered will be determined by a physician, in the light of the relevant circumstances, including the condition to be treated, the chosen route of administration, the actual compound administered, the age, weight, and response of the individual subject, the severity of the subject's symptoms, and the like.

For preparing solid compositions such as tablets, the principal active ingredient anticancer therapy is mixed with a pharmaceutical excipient or carrier to form a solid preformulation composition containing a homogeneous mixture of a compound of the present invention. When referring to these preformulation compositions as homogeneous, it is meant that the active ingredient is dispersed evenly throughout the composition so that the composition can be readily subdivided into equally effective unit dosage forms such as tablets, pills and capsules.

The tablets or pills of the present invention can be coated or otherwise compounded to provide a dosage form affording the advantage of prolonged action and to protect the anticancer therapies (such as an oligonucleotide) from acid hydrolysis in the stomach. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention can be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Parenteral routes of administration include but are not limited to direct injection into a central venous line, intravenous, intramuscular, intraperitoneal, intradermal, or subcutaneous injection. Formulations suitable for parenteral administration (e.g., an RNA molecule as described herein in a microcarrier formulation) are generally formulated in USP water or water for injection and may further comprise pH buffers, salts bulking agents, preservatives, and other pharmaceutically acceptable excipients. RNA molecules or DNA molecules as described herein, for example as microcarrier complexes or encapsulates, for parenteral injection may be formulated in pharmaceutically acceptable sterile isotonic solutions such as saline and phosphate buffered saline for injection.

Compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as described herein. The compositions can be administered by the oral or nasal respiratory route for local or systemic effect. Compositions in pharmaceutically acceptable solvents can be nebulized by use of inert gases. Nebulized solutions can be inhaled directly from the nebulizing device or the nebulizing device can be attached to a face mask tent, or intermittent positive pressure breathing machine. Solution, suspension, or powder compositions can also be administered, orally or nasally, from devices which deliver the formulation in an appropriate manner.

### V. Kits and Articles of Manufacture

Kits comprising a pharmaceutical composition comprising an isolated RNA molecule as described herein are also provided. For example, a kit can comprise unit dosage forms of the isolated RNA molecule, and a package insert containing instructions for use of the composition in treatment of a cancer. In some embodiments, the kit comprises a unit dosage form of the isolated RNA molecule, and at least one pharmaceutically acceptable vehicle. The instructions for use in the kit may be for treating a cancer. In some embodiments, the kit comprises the isolated RNA molecule or pharmaceutical composition comprising the isolated RNA molecule as described herein. In some embodiments, the kit comprises instructions for use of the isolated RNA molecule or pharmaceutical composition comprising the isolated RNA molecule in the treatment of cancer, such as, but not limited to, any of the cancers discussed above.

Pharmaceutical compositions (including, for example, formulations and unit dosages) comprising the isolated RNA molecules as described herein, can be prepared and placed in an appropriate container, and labeled for treatment of a cancer. Accordingly, provided is also an article of manufacture, such as a container comprising a unit dosage form of the isolated RNA molecule as described herein, and a label containing instructions for use of the isolated RNA molecule. In some embodiments, the article of manufacture is a container comprising a unit dosage form of the isolated RNA molecule, and at least one pharmaceutically acceptable vehicle. The article of manufacture may be a bottle, vial, ampoule, single-use disposable applicator, or the like, containing the pharmaceutical composition provided in the present disclosure. The container may be formed from a variety of materials, such as glass or plastic and in one aspect also contains a label on, or associated with, the container which indicates directions for use in the treatment of a cancer. It should be understood that the active ingredient may be packaged in any material capable of improving chemical and physical stability.

Any pharmaceutical composition provided in the present disclosure may be used in the articles of manufacture, the same as if each and every composition were specifically and individually listed for use in an article of manufacture.

### Examples

### Example 1: Knockdown of ASncmtRNA with Antisense Oligonucleotide Complementary to the Loop Region of the ASncmtRNA

In this study, expression of antisense non-coding chimeric mitochondrial RNA (ASncmtRNA) was knocked down by treatment with antisense oligonucleotide 1537S (ASO-1537S), complementary to the loop region of the ASncmtRNA.

### Materials and Methods

HeLa, HFK or SK-MEL-2 cells were cultured according to ATCC guidelines and maintained in a humidified cell culture chamber at 37° C. and 5% CO₂. ASO-1537S, having the sequence of 5′-CACCCACCCAAGAACAGG (SEQ ID NO:36) and control antisense oligonucleotide (ASO-C) having the sequence of 5′-AGGTGGAGTGGATTGGGG (SEQ ID NO:38) were synthesized by IDT or Invitrogen with 100% phosphorothioate linkages. Cells were seeded into a 12-well plate (Nunc) at 50,000 cells per well (HeLa, HFK) or 100,000 cells per well (SK-MEL-2) and transfected the next day with ASOs at 100 nM (HeLa, HFK) or 150 nM (SK-MEL-2) using 2.5 μl, 2.0 μl and 1.0 μl Lipofectamine 2000 (Invitrogen) respectively for HeLa, SK-MEL-2 and HFK cells, according to the manufacturer's directions, or left untreated. Transfection was allowed to proceed under normal culture conditions, as well as an untreated control sample, with cells harvested at desired time points up to 48 hours.

Cells were harvested and the RNA extracted with TRIzol Reagent (Invitrogen) (Villegas et al., Nucleic Acids Res., 2007, 35:7336-7347; Burzio et al., Proc. Natl. Acad. Sci. U.S.A., 2009, 106:9430-9434; Villota et al., J. Biol. Chem., 2012, 287:21303-21315). The RNA preparations were treated with TURBO DNA-free (Ambion) to eliminate DNA contamination. Convention RT-PCR was carried out with 50-100 ng RNA, 50 ng random hexamers, 0.5 mM each dNTP, 5 mM DTT, 2 U/μl Rnase-out (Invitrogen) and 200 U reverse 25 transcriptase (M-MLV, Invitrogen). Reactions were incubated at 25° C. for 10 minutes, 37° C. for 50 minutes and 65° C. for 10 minutes. PCR was carried out in 50 μl containing 2 μl cDNA, 0.4 mM each dNTP, 1.5 mM MgCl₂, 2 U GoTaq (Promega) and 1 μM each forward or reverse primer (ASncmtRNA-1 (AS-1), ASncmtRNA-2 (AS-2) and 18S as loading control). The primers used are provided in the following table.

| | | |
|---|---|---|
| AS-1 | Forward | 5′-TAGGGATAACAGCGCAATCCTATT |
| | | (SEQ ID NO: 39) |
| | Reverse | 5′-CACACCCACCCAAGAACAGGGAGGA |
| | | (SEQ ID NO: 40) |
| AS-2 | Forward | 5′-ACCGTGCAAAGGTAGCATAATCA |
| | | (SEQ ID NO: 41) |
| | Reverse | 5′-ACCCACCCAAGAACAGG |
| | | (SEQ ID NO: 42) |
| 18S | Forward | 5′-AGTGGACTCATTCCAATTA |
| | | (SEQ ID NO: 43) |
| | Reverse | 5′-GATGCGTGCATTTAT |
| | | (SEQ ID NO: 44) |

The amplification protocol consisted of 5 minutes at 94° C., 30 cycles of 94° C., 58° C., and 72° C. for 1 minute each followed by 10 minutes at 72° C. The 18S samples were only amplified for 15 cycles. Samples were loaded onto SDS-PAGE with 100 bp-ladder to approximate size.

### Results

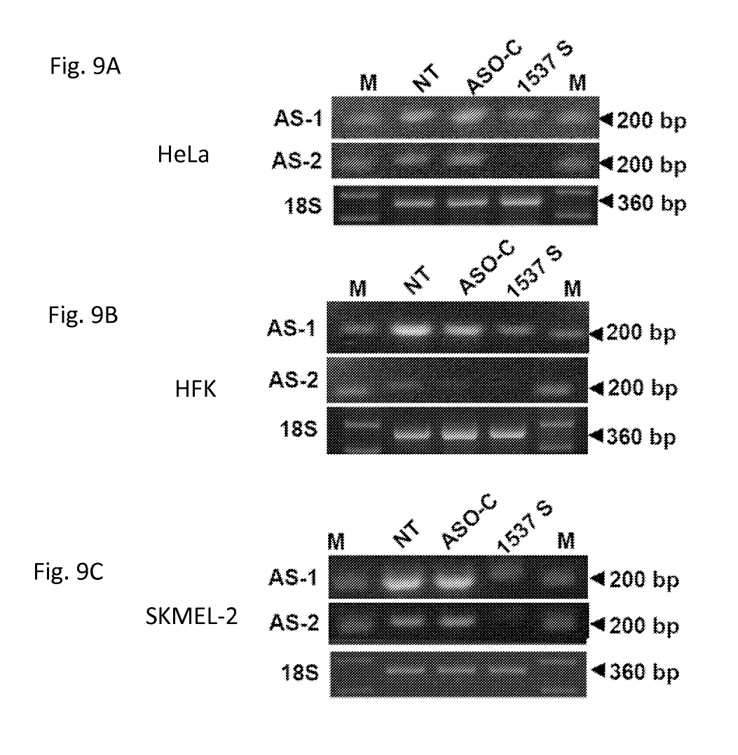

FIGS. 9A, 9B and 9C show the knockdown of AS-1 and AS-2 in the two cancer cell lines, HeLa (9A) and SK-MEL-2 (9C) by ASO-1537S relative to ASO-C and untreated samples as compared to normal HFK cells (9B).

### Example 2: Inhibition of HeLa Cell Proliferation and Induction of HeLa Cell Death by Knockdown of ASncmtRNA with Antisense Oligonucleotide Complementary to the Loop Region of the ASncmtRNA

In this study, knockdown of expression of antisense non-coding chimeric mitochondrial RNA (ASncmtRNA) with antisense oligonucleotide 1537S (ASO-1537S) was shown to induce cell death in HeLa cells.

### Materials and Methods

HeLa cells were cultured and transfected with ASO-C, ASO-1537S, ASO-1537S 5′-Alexa Fluor® 488, or untreated, as described in Example 1.

Triplicate samples of ASO-1537S, ASO-C or untreated cells were harvested at 24, 36 and 48 hours, and cells were counted to assess cell proliferation. Similarly treated cells were prepared in triplicate and harvested at 48 hours to assess cell proliferation using Click-iT® EdU Alexa Fluor® 488 Kit (Invitrogen), following a 2 hour EdU (5-ethynyl-2′-deoxyuridine) pulse, according to manufacturer's directions. The cells were co-stained with DAPI to assess the total number of cells, where EdU incorporation was measured as EdU positive cells versus total cells. Samples treated with ASO-1537S 5′-Alexa Fluor® 488 labeled were harvested at 48 hours. Harvested cells with fluorescent marker were analyzed on an Olympus BX-51 fluorescence microscope. In another set of ASO-1537S, ASO-C and untreated cells, the cells were harvested at 48 hours and viewed on phase microscopy to assess cell detachment from substrate. In another study, ASO-1537S and ASO-C were transfected at 25, 50 and 100 nM along with untreated cells. The cells were harvested at 48 hours and stained with Trypan blue (Tb) and counted. The % Tb-positive cells indicated the amount of cell death.

### Results

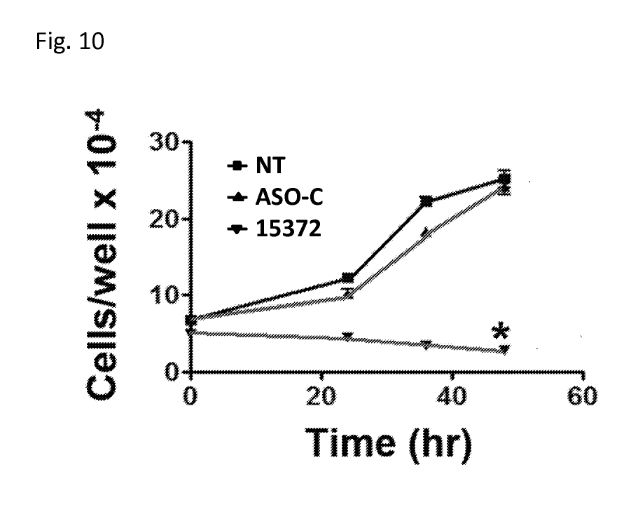

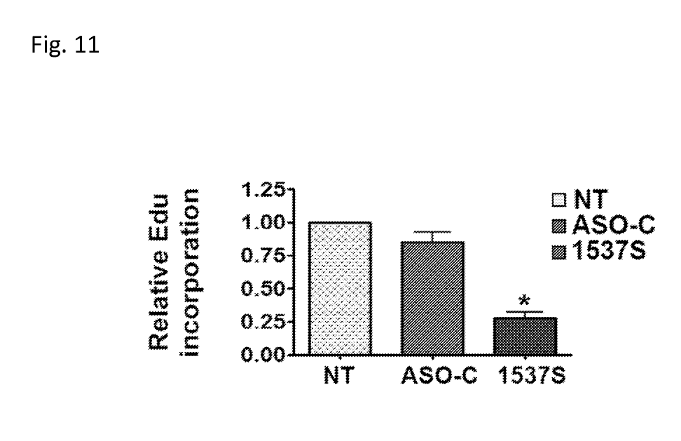

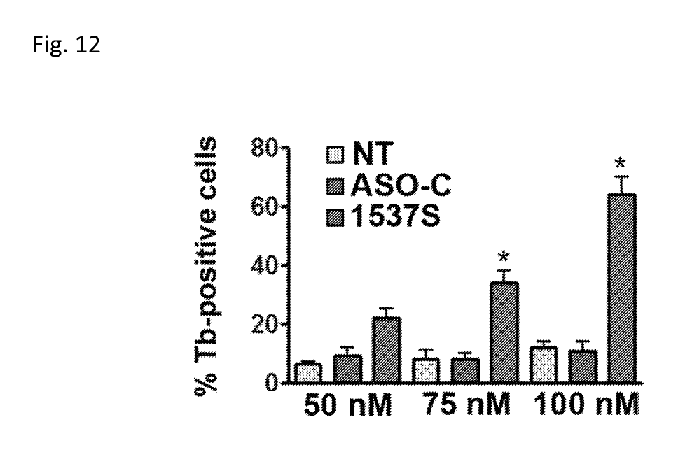

The cells transfected with ASO-1537S 5′-Alexa Fluor® 488 showed greater than 90% of the cells were transfected at 24 hours. FIG. 10 shows statistically significant inhibition of cell proliferation in the ASO-1537S transfected cells compared to ASO-C and untreated cells (mean±SEM; *p<0.01). FIG. 11 shows that EdU incorporation for ASO-1537S treated cells was significantly reduced as compared to ASO-C or untreated samples (*p<0.01). The results of the analysis of detached cells showed that ASO-1537S induced massive cell detachment not observed in the ASO-C or untreated cells. FIG. 12 shows the % Tb-positive cells dose response of cells harvested at 48 hours, where the ASO-1537S induced cell death was over 60% at 100 nM (*p<0.01).

### Example 3: Induction of HeLa Cell Death by Knockdown of ASncmtRNA with Antisense Oligonucleotide Complementary to the 3′ Single-Stranded Region of the ASncmtRNA

In this study, expression of antisense non-coding chimeric mitochondrial RNA (ASncmtRNA) was knocked down by treatment with antisense oligonucleotides complementary to the 3′ single-stranded region of the ASncmtRNA: 1107S (ASO-1107S), 522S (ASO-522S) or 126S (ASO-126S).

### Materials and Methods

HeLa cells were cultured as described in Example 1. ASO-1107S having the sequence of 5′-GTCCTAAACTACCAAACC (SEQ ID NO:35), ASO-552S having the sequence of 5′-TACCTAAAAAATCCCAAACA (SEQ ID NO:34), and ASO-126S having the sequence of 5′-AGATGAAAAATTATAACCAA (SEQ ID NO:33) were synthesized by IDT or Invitrogen with 100% phosphorothioate linkages. HeLa cells were cultured and transfected as described in Example 1, with 100 nM of ASO-1107S, ASO-522S, ASO-126S, ASO-C, or untreated. The cells were harvested at 48 hours and stained with Trypan blue (Tb) and counted. The % Tb-positive cells indicated the amount of cell death.

### Results

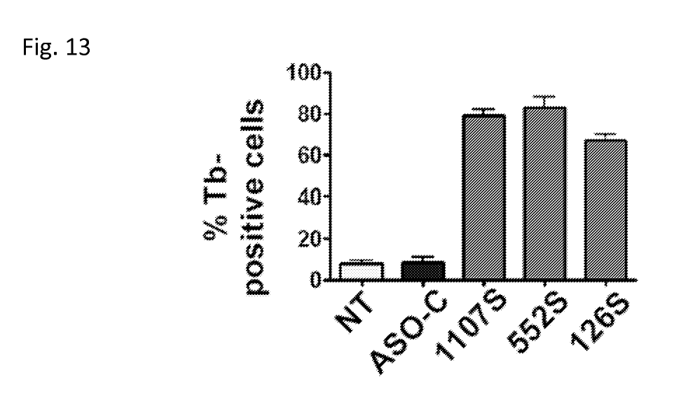

FIG. 13 shows the % Tb-positive cells harvested at 48 hours, where the ASO-1107S, ASO-552S or ASO-126S induced cell death was over 70% (*p<0.01).

### Example 4: Transfection of Various Cell Lines with ASO-1537S 5′-Alexa Fluor® 488

In this study, transfection of a variety of cancer cell lines as well as normal cell lines with ASO-1537S 5′-Alexa Fluor® 488 is demonstrated.

### Materials and Methods

Pooled neonatal human foreskin keratinocytes (HFK) were purchased from Lonza (Basel, Switzerland) and cultured in Keratinocyte Serum-free Medium (KSFM, Invitrogen). All other cell lines were obtained from ATCC and cultured per ATCC guidelines. All cell cultures were maintained in a humidified cell culture chamber at 37° C. and 5% CO₂. Cancer cell lines of HPV 16-transformed cervix (SiHa), breast carcinoma (MDA-MB-231), prostate carcinoma (DU145), lung carcinoma (H292), melanoma (SK-MEL-2), renal carcinoma (A498), and ovarian carcinoma (OVCAR-3), as well as normal cell lines of HFK, human renal epithelial cells (HREC), and human melanocytes (HnEM) were cultured at 50,000 cells per well and transfected as described in Example 1 with ASO-1537S 5′-Alexa Fluor® 488, with amounts of ASO and Lipofectamine provided in the following table (additional cell lines included for use in subsequent Examples).

| | nM | μl of |
|---|---|---|
| Cell line | ASO-1537S | Lipofectamine |
| SiHa; MDA-MB-231; MCF7; | 100 | 2.5 |
| DU145; H292; HUVEC | | |
| HFK | 100 | 1.0 |
| PC3 | 100 | 2.0 |
| SK-MEL-2; HREC | 150 | 2.0 |
| HepG2; U87; Caco-2 | 150 | 2.5 |
| A498 | 150 | 4.0 |
| HnEM | 150 | 4.0 (Lipofectin) |
| OVCAR-3 | 200 | 2.0 |

The cells were harvested at 24 hours and analyzed on an Olympus BX-51 fluorescence microscope.

### Results

All cell lines demonstrated greater than 90% transfection of the cells at 24 hours post transfection.

### Example 5: Treatment with ASO-1537S does not Affect Normal Cells

In this example, human umbilical vein endothelial cells (HUVEC), human renal epithelial cells (HREC) and human melanocytes (HnEM) cells were transfected with ASO-1537S and assessed by Trypan blue staining.

### Materials and Methods

HUVEC cells were cultured in M199 media (Gibco) supplemented with 20% FCS, 50 μg/ml heparin, 50 μg/ml endothelial cell growth supplement (ECGS; Calbiochem) and Pen/Strep (50 U/ml penicillin, 50 μg/ml streptomycin), HREC and HnEM were cultured as described in Example 4. HUVEC, HREC and HnEM cells were cultured at 50,000 cells per well and transfected with ASO-1537S or ASO-C as described in Example 3, or untreated, all in triplicate. At 48 hours post transfection, cells were harvested, stained with Trypan blue, and counted.

### Results

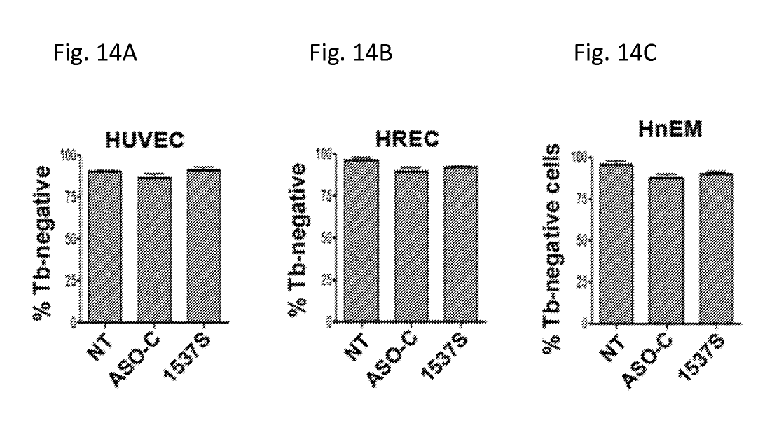

All of the ASO-1537S treated samples were comparable to control and untreated cells, with over 90% of cells Tb-negative, as seen in FIGS. 14A, 14B and 14C.

### Example 6: Mitochondrial Alterations and Apoptosis Induced by ASO-1537S

In this example, HeLa cells were treated with ASO-1537S and the mitochondrial membrane potential (ΔΨm) was measured.

### Materials and Methods

HeLa cells were cultured at 50,000 cells per well. To assess ΔΨm, at 24 hours post transfection, loaded with 20 nM tetramethylrhodamine methyl ester (TMRM, Molecular Probes) for 15 minutes at 37° C., harvested and analyzed by flow cytometry on a BDS-FACS Canto Flow Cytometer. As a positive control, cells were treated with 10 μM carbonyl cyanide 3-chlorophenylhydrazone for 30 minutes at 37° C. (CCCP, Sigma-Aldrich) prior to staining with TMRM. DU145, MDA-MB-231 and H292 cells were similarly assayed for ΔΨm, transfecting as described in Example 4.

### Results

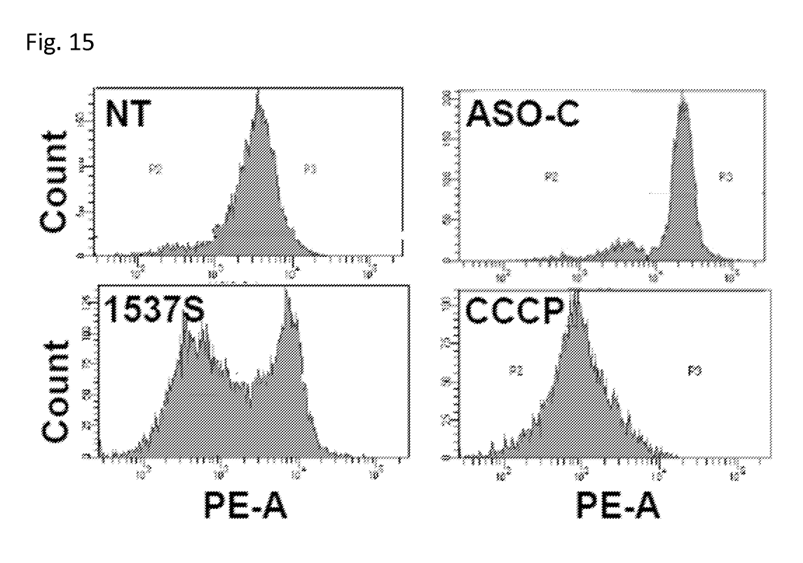

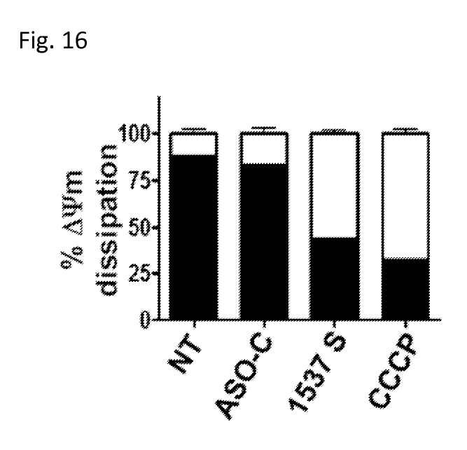

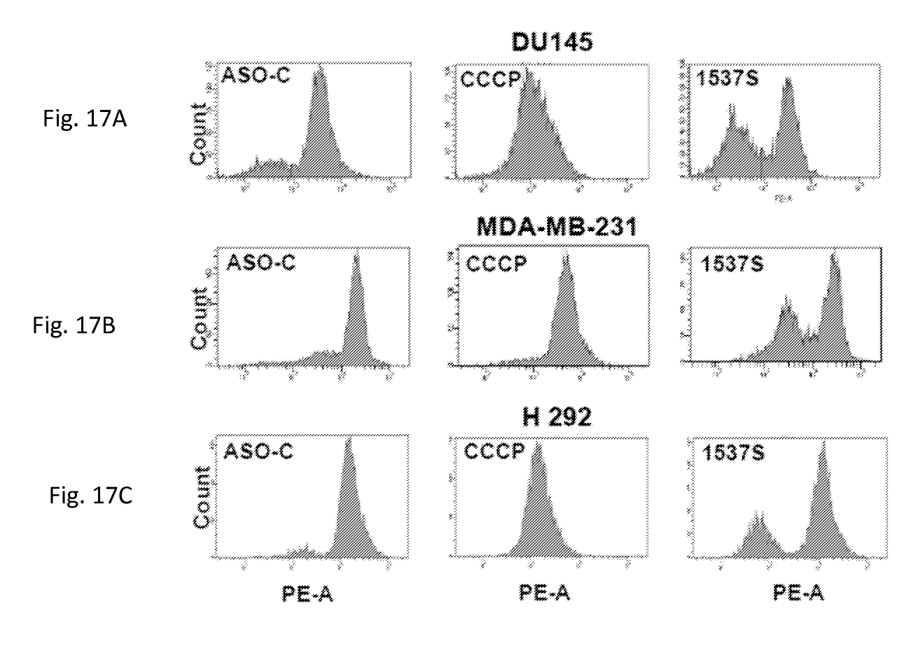

The ΔΨm of ASO-1537S treated samples was comparable to the positive control. Samples assayed in triplicate showed that ASO-1537S induced about 55% dissipation of ΔΨm compared to 70% for CCCP and about 10-15% for control and untreated cells. FIG. 15 shows the Flow Cytometry plots for HeLa cells, and FIG. 16 shows the % ΔΨm (white portion of bar) for each sample, in triplicate. The Flow Cytometry results for DU145, MDA-MD-231 and H292 are shown in FIG. 17A-C. Thus, the ASO-1537S treated samples induced a marked disruption of the ΔΨm which is an early hallmark of apoptosis.

### Example 7: Release of Cytochrome c from Mitochondria and Activation of Caspases

Dissipation of ΔΨm induces the release of cytochrome c, followed by activation of caspases (Heerdt et al., Cancer Res., 66:1591-1596; Houston et al., Int. J. Cell Biol., 2011, article 978583; Gottlieb et al., Cell Death Differ., 2003, 10:709-717). In this study, HeLa cells treated with ASO-1537S or staurosporine (STP, apoptosis control) are assessed for the release of cytochrome c into the cytoplasm and for activation of caspases.

### Materials and Methods

HeLa cells were cultured at 50,000 cells per well and transfected with 100 nM ASO-1537S or ASO-C as described in Example 1, treated with staurosporine, or untreated. After 24 hours, cells were harvested and washed in ice cold PBS, then centrifuged at 1000×g for 10 minutes at room temperature. The pellets were suspended in Radio-Immunoprecipitation Assay buffer (RIPA; 10 mM Tris-HCl pH 7.4, 1% sodium deoxycholate, 1% Triton X-100, 0.1% sodium dodecyl sulfate), containing 1 mM PMSF and protease inhibitor cocktail (Sigma-Aldrich). Protein concentration was quantified with the Bradford microplate-system Gen5™ EPOCH (BioTeK) and samples were analyzed by Western Blot. The samples were loaded at 30 μg protein per lane on SDS-PAGE and transferred to polyvinylidine difluoride (PVDF) membranes. Membranes were probed with rabbit polyclonal antibodies against cytochrome c (Cell Signaling; 1:1000), with mouse monoclonal anti-β-actin (Sigma-Aldrich; 1:4000) as a loading control. Blots were revealed with peroxidase-labeled anit-mouse or anti-rabbit IgG (Calbiochem; 1:5000). The blots were detected with the EZ-ECL system (Biological Industries).

Similarly prepared samples were assayed for caspase activation, adding FITC-VAD-fmk (Promega) as a fluorogenic caspase inhibitor which binds to activated caspases (Garcia-Calvo et al., J. Biol. Chem., 1998, 273:32608-32613). In addition to STP treated control, cells are treated with 100 μM H₂O₂ as a positive control for necrosis. The FITC-VAD-fmk was added after transfection, STP treatment, or H₂O₂ treatment at 10 μM and incubated for 20 minutes at 37° C. Cells were harvested, washed in PBS and fixed in 3.7% p-formaldehyde for 15 minutes at room temperature, and fluorescent images were obtained. In order to confirm that caspase inactivation was involved in cell death, inhibition of apoptosis was carried out with the non-fluorescent caspase inhibitor z-VAD-fmk (Promega), added 2 hours prior to transfection or STP treatment at a concentration of 25 μM for HeLa cells. Cell death was assayed by propidium iodide staining.

### Results

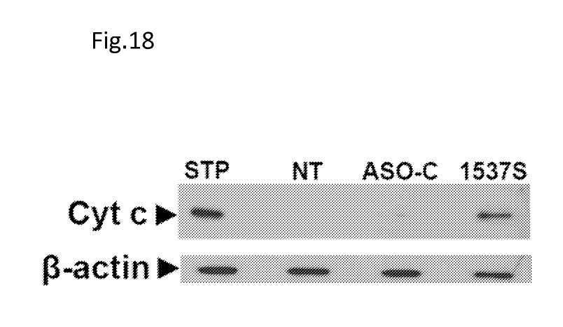

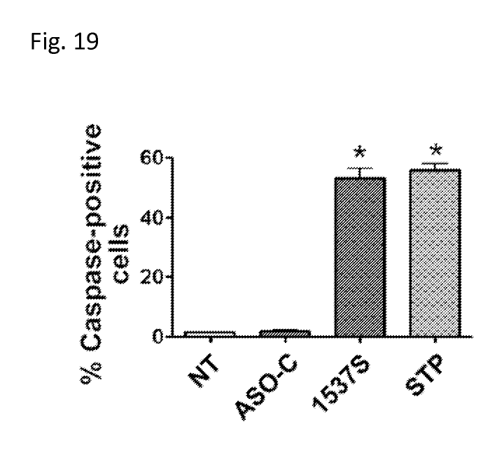

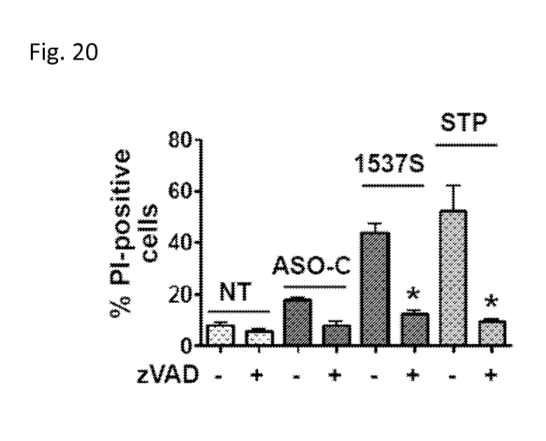

The western blot analysis for the presence of cytochrome c showed that only those cells treated with ASO-1537S or STP had cytocrhome c in the cytosolic fraction (FIG. 18). FIG. 19 shows that about 50% of the cells treated with ASO-1537S or the apoptosis positive control STP contained activated caspases, with very little in the control and untreated cells. FIG. 20 shows a statistically significant reduction in the % PI positive cells of z-VAD-fmk pre-treated cells for the ASO-1537S and STP treated cells, indicating caspase activation involvment in cell death.

### Example 8: Measurement of Annexin V in ASO-1537S Treated HeLa Cells

In this experiment, the translocation of phosphatidylserine to the outer layer of the plasma membrane, which is an indication of apoptosis, was assessed. Phosphatidylserine on the surface of cells was detected by binding to fluorescent labeled Annexin V.

### Materials and Methods

HeLa cells were transfected with ASO-1537S, ASO-C, STP or untreated and cultured as described in Examples 1 and 7. After 24 hours post treatment, cells were labeled with Annexin V-Alexa fluor 488 (APOtarget kit, Invitrogen) according the manufacturer's directions, and stained with propidium iodide.

### Results

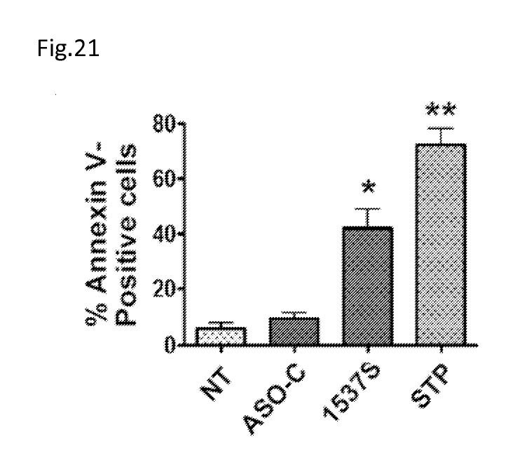

Annexin V positive cells were observed for both the STP and ASO-1537S treated cells. The % Annexin V positive cells are shown in FIG. 21 for samples assayed in triplicate, showing a statistically significant fraction of 40% (*p<0.01) and 70% (*p<0.005) Annexin V positive cells for ASO-1537S and STP treated cells, respectively, with <10% Annexin V positive cells in the untreated and ASO-C control treated cells.

### Example 9: Assessment of DNA Fragmentation in ASO-1537S Treated Cells

In this experiment, DNA fragmentation is measured by terminal deoxynucleotidyl transferase dUTP nick end labeling (TUNEL assay) in cancer cell lines of MCF7 (breast carinoma), PC3 (prostate carcinoma), HepG2 (hepatoma), Caco-2 (colon carcinoma), U87 (glioblastoma), SiHa, MDA-MB-231, DU145, SK-MEL-2, A498, OVCAR-3 and HeLa and in normal cell line HFK.

### Materials and Methods

Cell lines were transfected with ASO-C, ASO-1537S or untreated and cultured as described in Example 4. HeLa and HFK cells were also treated with Dnase I as a positive control. After 48 hours, cells were harvested, fixed, and subjected to TUNEL assay. The samples were evaluated using Dead End™ Fluorometric TUNEL kit (Promega) according to the manufacturer's directions, resulting in fluorscein-12-dUTP labeling of fragmented nucleic acid. Cells were also stained with propidium iodide (PI) or 4′,6-diamidino-2-phenylindole (DAPI) to distinguish the cells labeled by the TUNEL assay from unlabeled cells.

### Results

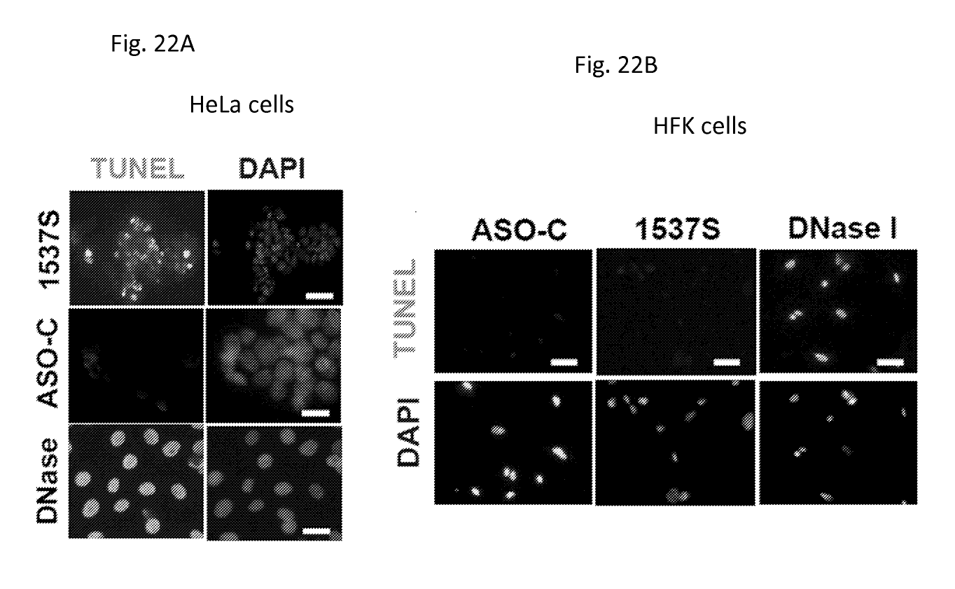

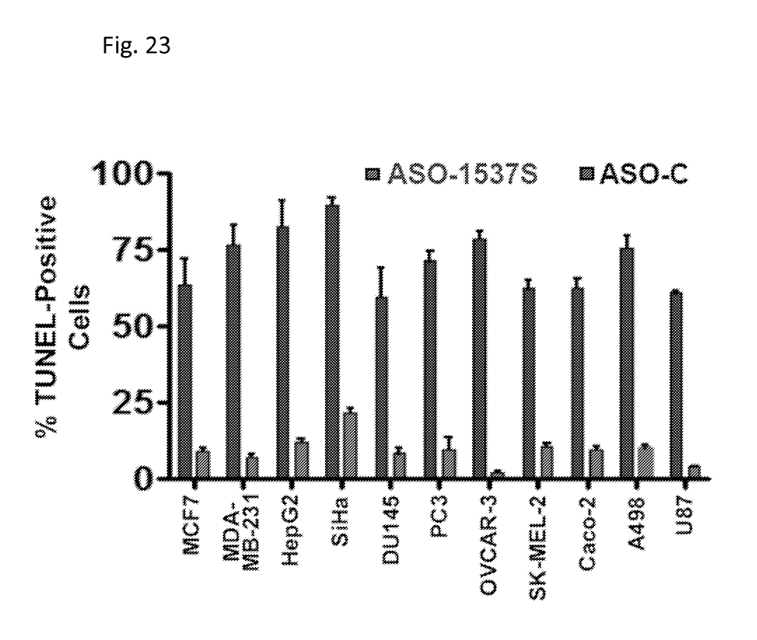

FIG. 22A shows HeLa cells stained with fluorescein-12-dUTP and DAPI, where only the positive control and ASO-1537S are stained with fluorescein, indicating considerable DNA fragmentation not observed in the ASO-C treated samples. By comparison, FIG. 22B shows that HFK cells are not affected by ASO-1537S treatment, as only the DNase I treated samples were stained with fluorescein. The additional cancer cell lines were stained with PI, and the % TUNEL-positive cells are shown in FIG. 23. In all cancer cell lines, triplicate analysis showed statistically significant fraction (60-80%) of cells were TUNEL-positive compared to ASO-C treated cells.

### Example 10: Assessment of Sub-G1 Fraction in ASO-1537S Treated Cells

In this experiment, the fraction of sub-G1 phase cells is assessed as a measure of hypodiploid events, indicative of apoptosis.

### Materials and Methods

HeLa or HFK cell lines were transfected with ASO-C, ASO-1537S, or untreated and cultured at 100,000 cells per well. HeLa and HFK cells were also treated with STP at a concentration of 5 μM as a positive control for apoptosis. After 24 hours (HeLa) or 48 hours (HFK), cells were harvested, centrifuged at 600×g for 5 minutes and the pellets were suspended in 100% ethanol and stored at −20° C. for 24 hours. Cells were then treated with 1 mg/ml Rnase A for 1 hour at room temperature. The cells were stained with PI and assessed by Flow Cytometry.

### Results

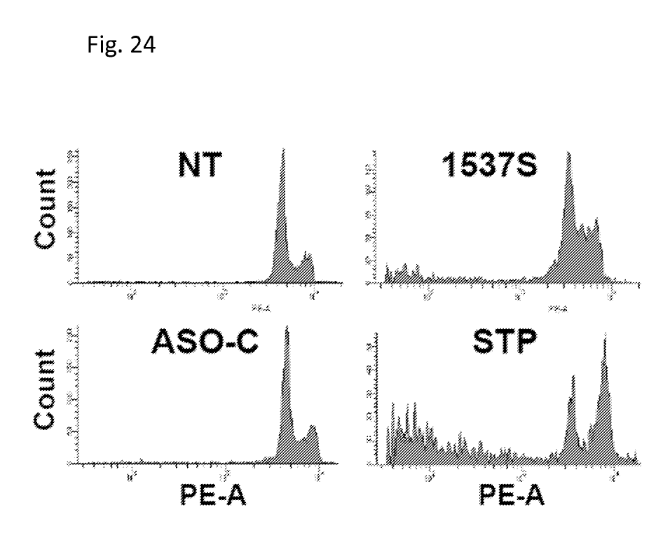

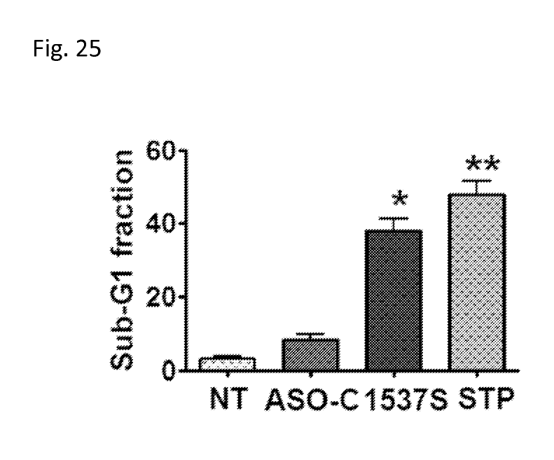

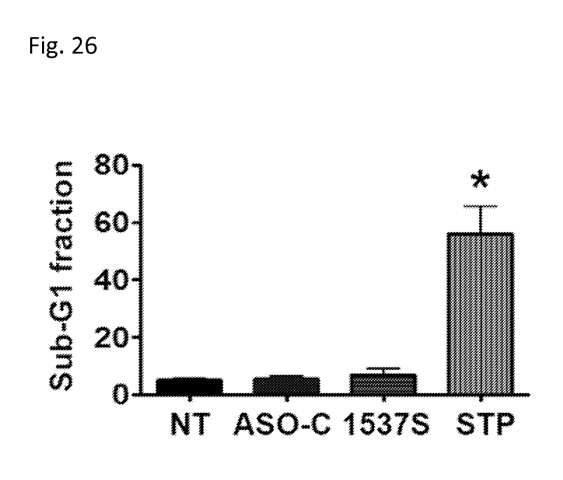

FIG. 24 shows the Flow Cytometry results for HeLa cells, indicating an increase in the sub-G1 fraction for the ASO-1537S and positive control STP treated cells, but not in the ASO-C or untreated cells. An analysis of samples in triplicate of the percent of sub-G1 in HeLa cells shows a significant sub-G1 fraction in the ASO-1537S treated (45%, *p<0.01) that is comparable to STP treated (50%, **p<0.005) (FIG. 25). FIG. 26 shows the percent of sub-G1 fraction in HFK cells, where only the STP treated cells show a significant sub-G1 fraction increase (55%, *p<0.01), with the ASO-1537S similar to ASO-C and untreated cells. This again demonstrates ASO-1537S treatment results in apoptosis of cancer cells, without affecting normal cells.

### Example 11: Assessment of Anchorage-Independent Growth in ASO-1537S Treated Cells

In this experiment, the colony formation of SK-MEL-2, OVCAR-3, HeLa and SiHa cancer cells in soft agar is assessed as a measure of anchorage independent-growth. This is considered a parameter of tumorogenicity (Bertotti et al., J. Cell Biol., 2006, 175:993-1003).

### Materials and Methods

SK-MEL-2, OVCAR-3, HeLa and SiHa were transfected with ASO-C, ASO-1537S or untreated and cultured as described in Example 4. Cells were harvested after 48 hours, counted and seeded in 12-well plates on soft agar in triplicate, with 200 cells per well for HeLa and SiHa, 500 cells per well for OVCAR-3 and 2000 cells per well for SK-MEL-2, as determined by Trypan blue exclusion. Colonies measuring over 50 μm were counted after 2-3 w.

### Results

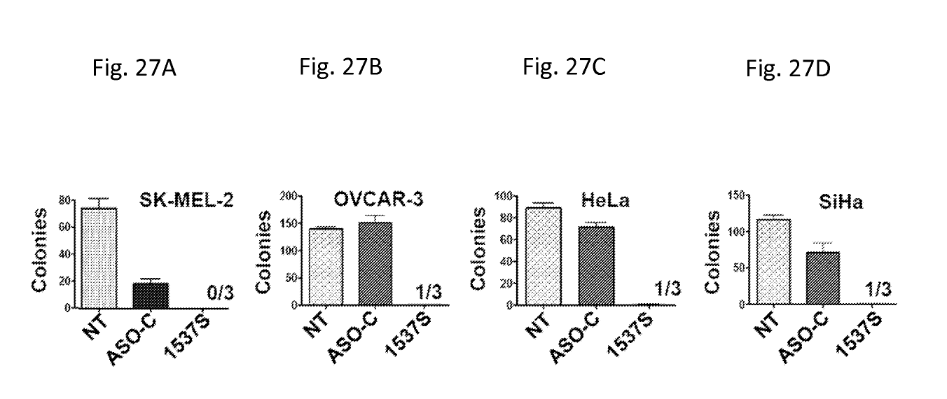

FIG. 27A-D shows the number of colonies over 50 μm, averages for untreated and ASO-C treated cells, while the number for ASO-1537S treated cells is the total number of colonies in all three wells. This very clearly shows that ASO-1537S inhibits anchorage-independent growth.

### Example 12: Down Regulation of Survivin in ASO-1537S Treated SK-MEL-2 Cells

In this experiment, ASO-1537S treated, control treated or untreated SK-MEL-2 cells are assessed for survivin expression by western blot analysis.

### Materials and Methods

SK-MEL-2 were transfected with 150 nM of ASO-C or ASO-1537S or untreated and cultured for 24 hours as described in Example 4. Harvested cells were processed for western blot analysis as described in Example 7, using an antibody against survivin (rabbit polyclonal; R&D systems; 1:1000). This was repeated in triplicate and bands were quantitated by measuring pixel intensity of each band using ImageJ software (NIH), normalized to β-actin levels as loading control. Additional samples were prepared for ASO-1537S treated cells and harvested at 3, 8, and 22 hours post transfection, and the survivin level was assessed relative to an untreated control.

To assess whether the survivin down regulation was due to proteosomal degradation, SK-MEL-2 cells were transfected with ASO-1537S or ASO-C with or without treatment with 26S proteosome inhibitor MG132 (Lin et al., J. Biol. Chem., 283:21074-21083, 2008). To assess whether survivin was degraded by activated caspases (Igarashi et al., Nucleic Acids Res., 35:D546-9, 2007), SKMEL-2 cells were transfected with ASO-1537S or ASO-C with or without the caspase inhibitor z-VAD-fmk.

### Results

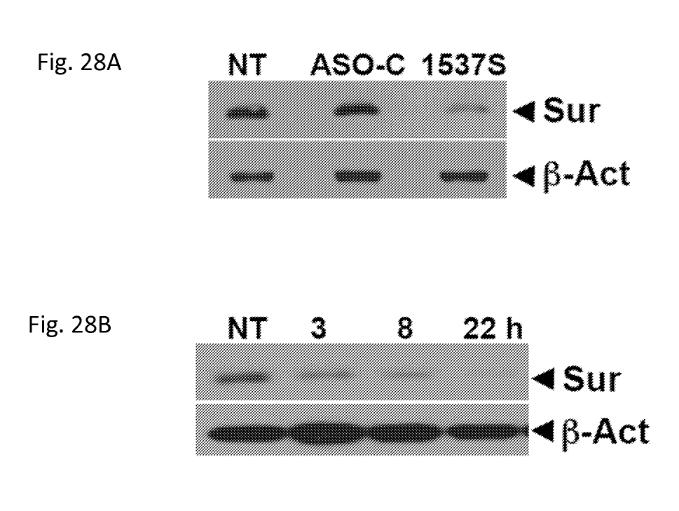

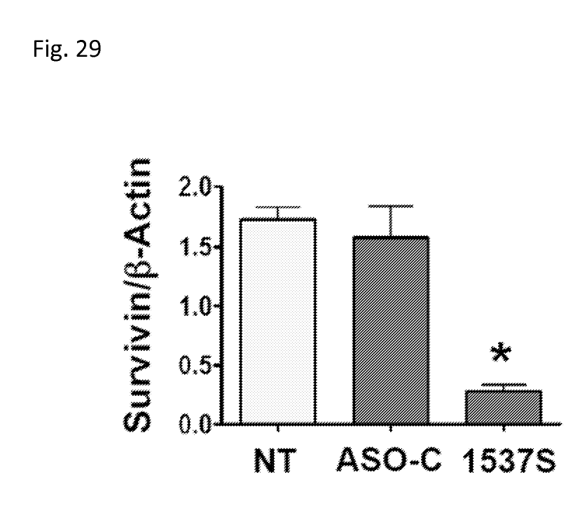

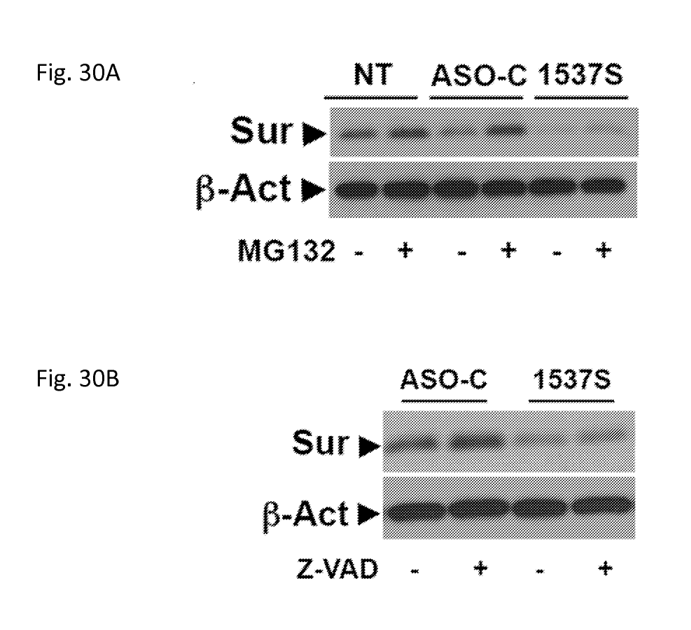

FIG. 28A shows a marked inhibition of survivin expression with ASO-1537S treated cells at 24 hours, relative to control and untreated cells. FIG. 28B shows the inhibition of survivin expression even at 3 hours post transfection, with increased inhibition levels at later time points. FIG. 29 shows densitometric analysis of blots from three independent experiments, with the survivin bands normalized to β-actin. The survivin level was decreased by 80% at 24 hours (*p<0.01). FIGS. 30A and 30B shows the inhibition of survivin expression with or without MG132 treatment or z-VAD-fmk treatment, respectively, suggesting that neither proteosomal degradation nor activated caspase are involved in the inhibition of survivin expression.

### Example 13: Down Regulation of Survivin in ASO-1537S Treated Cells not Due to mRNA Degradation

In this experiment, the relative level of mRNA in cells treated with ASO-1537S was compared to control treated SK-MEL-2 cells.

### Materials and Methods

SK-MEL-2 were transfected with 150 nM of ASO-C or ASO-1537S or untreated and assessed by RT-PCR. cDNA was synthesized with the Affinity Script QPCR cDNA Synthesis Kit (Agilent Technologies) using 500 ng RNA and 250 ng random hexamers (Invitrogen). Reactions were incubated 10 minutes at 25° C., 1 hour at 45° C. and 5 minutes at 95° C. RNase H (2U) was added and samples were incubated at 37° C. for 20 minutes. Real-time PCR (qPCR) for survivin was carried out on a Stratagene Mx3000P™ Real-time PCR System (Agilent Technologies) with 3 μl of a 1:5 cDNA dilution, 1× GoTaq Flexi Buffer, 2 mM MgCl₂, 0.4 mM each dNTP, 2.5 U GoTaq DNA polymerase, 0.5 μM each forward and reverse primer and 0.25 μM Taqman probe and in a volume of 25 μl. Cycle parameters were: 95° C. for 2 minutes and 40 cycles of 95° C. for 15 seconds, 54° C. for 15 seconds and 62° C. for 45 seconds. The primers and probes used are provided in the following table, where RPL27 mRNA and 18S rRNA are used to normalize the results.

| | | |
|---|---|---|
| Survivin | Forward | 5′-ATGGGTGCCCCGACGT |
| | | (SEQ ID NO: 46) |
| | Reverse | 5′-AATGTAGAGATGCGGTGGTCCTT |
| | | (SEQ ID NO: 47) |
| | Probe | 5′-CCCCTGCCTGGCAGCCCTTTC |
| | | (SEQ ID NO: 48) |
| RPL27 | Forward | 5′-AATCACCTAATGCCCAC |
| | | (SEQ ID NO: 49) |
| | Reverse | 5′-TGTTCTTGCCTGTCTTG |
| | | (SEQ ID NO: 50) |
| | Probe | 5′-CAGAGATCCTGCTCTTAAACGC |
| | | (SEQ ID NO: 51) |
| 18S | Forward | 5′-GTAACCCGTTGAACCCCATT |
| | | (SEQ ID NO: 52) |
| | Reverse | 5′-CATCCAATCGGTAGTAGCG |
| | | (SEQ ID NO: 53) |
| | Probe | 5′-AGTAAGTGCGGGTCATAAGCTTGCGT |
| | | (SEQ ID NO: 54) |

### Results

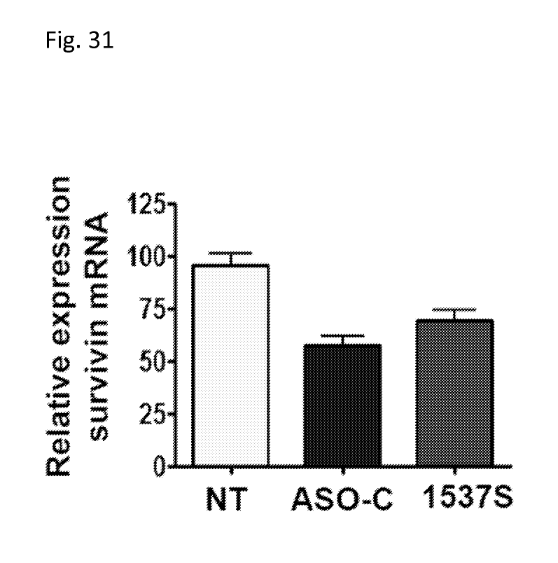

FIG. 31 shows the relative expression of survivin mRNA in ASO-1537S or ASO-C treated and untreated SK-MEL-2 cells. The relative survivin mRNA in ASO-1537S treated cells is comparable to untreated and control cells, suggesting that the decrease in survivin protein expression is not due to mRNA degradation.

### Example 14: Down Regulation of Survivin in PC3, OVCAR-3 and H292 Cells Treated with ASO-1537S

In this experiment, ASO-1537S treated, control treated or untreated PC3, OVCAR-3 or H292 cells are assessed for survivin expression by western blot analysis.

### Materials and Methods

PC3, OVCAR-3 or H292 cells were transfected with ASO-C or ASO-1537S or untreated and cultured for 24 hours as described in Example 4. Samples were assayed for survivin levels as described in Example 12 and similarly for Bcl-2 expression levels using an antibody against Bcl-2 (rabbit polyclonal; Abcam; 1:1000).

### Results

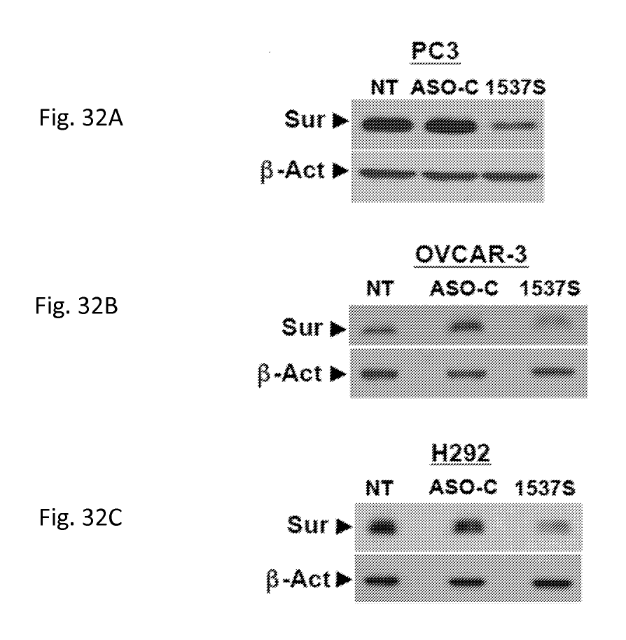

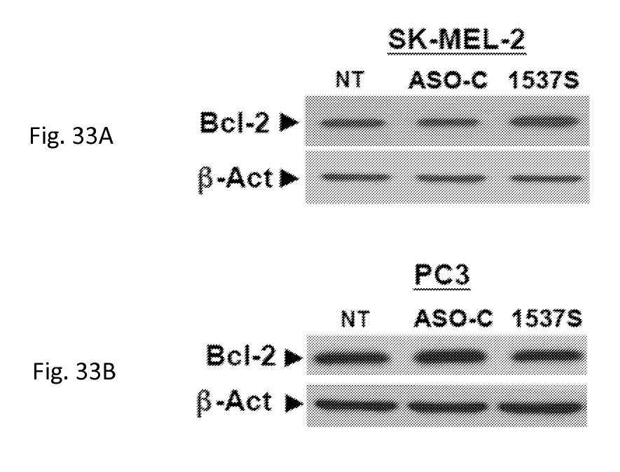

FIG. 32A-C shows that survivin expression is similarly inhibited in ASO-1537S treated PC3, OVCAR-3 and H292 cells relative to control and untreated cells. FIGS. 33A and 33B shows no noticable effect of ASO-1537S treatment of SK-MEL-2 or PC3 cells on the expression of Bcl-2 relative to control of untreated cells.

### Example 15: Down Regulation of Other Proteins in SK-MEL-2 Cells Treated with ASO-1537S or ASO-226S

In this experiment, SK-MEL-2 cells were treated with ASO-1537S, ASO-226S (complementary to the 3′ single-stranded region of the ASncmtRNA), ASO-C or untreated and levels of Cyclin D1, Cyclin B1, N-cadherin and caveolin were assessed by western blot analysis. Cyclin D1 is required for transition of cells from phase G1 to S, and reduction of Cyclin D1 levels results in increase in G1 phase cells. Cyclin B1 is a known inductor of mitosis, and inhibition of mitosis leads to arrest in G2 phase of the cycle and increased cell death. N-cadherin and caveolin are involved in cell migration, cell invasion and metastasis, and their down regulation inhibits these cancer cell properties.

### Materials and Methods

ASO-226S having the sequence of 5′-TAAGACCCCCGAAACCAGAC (SEQ ID NO:45) was synthesized with 100% phosphorothioate linkages (IDT or Invitrogen or Biosearch Inc.). SK-MEL-2 cells were transfected with 150 nM of ASO-C, or ASO-1537S, or untreated and cultured for 24 hours as described in Example 4. Cyclin B1 was also assessed in cells transfected with ASO-226S. Harvested cells were processed for western blot analysis as described in Example 7, using an antibody against cyclin D1 (monoclonal; BC PHarmagen; 1:250), cyclin B1 (monoclonal; BD Pharmagen; 1:500), N-cadherin (rabbit polyclonal; Thermo Scientific; 1:1000), or caveolin (rabbit polyclonal; Abcam; 1:1000). Each was repeated in triplicate and bands were quantitated by measuring pixel intensity of each band using ImageJ software (NIH), normalized to β-actin levels as loading control.

### Results

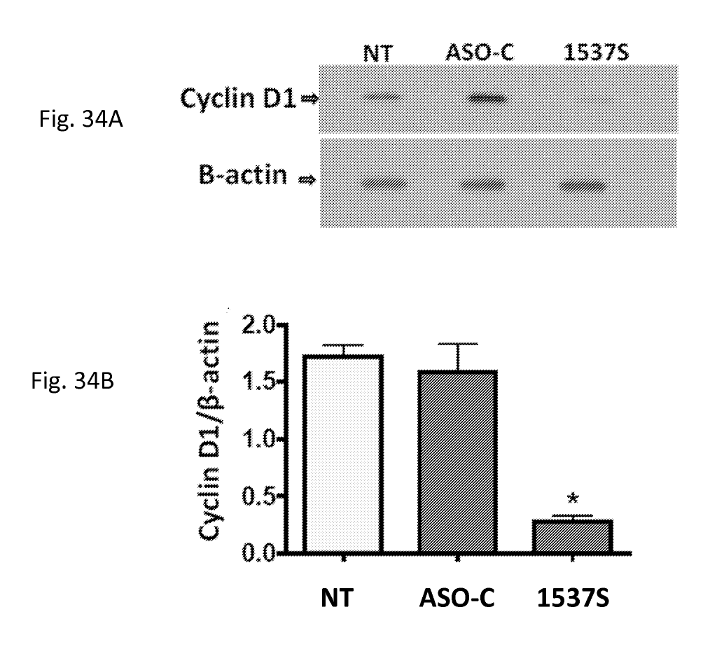

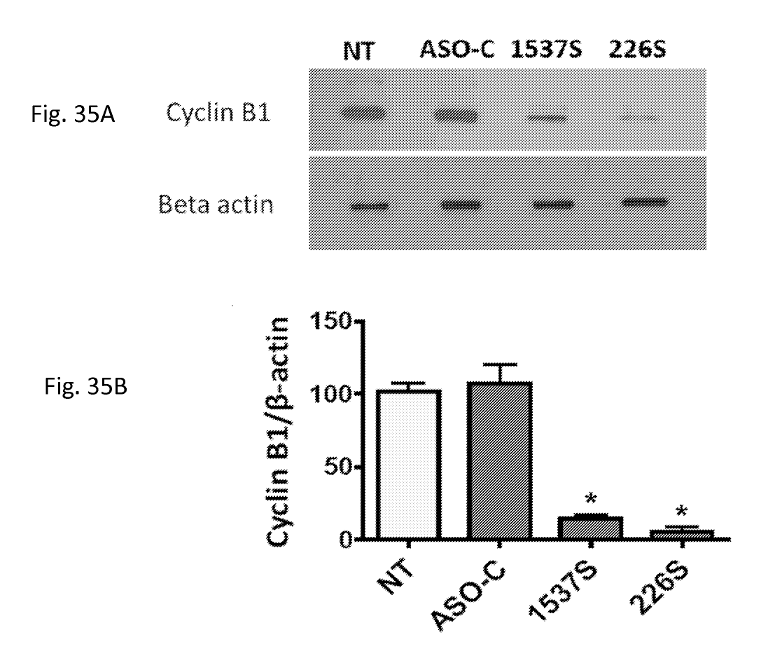

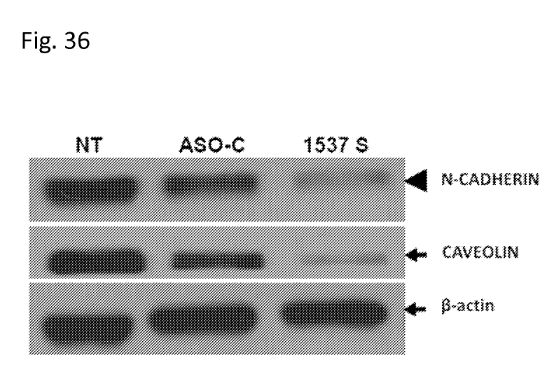

FIG. 34A shows the western blot results for cyclin D1, with band quantitation shown in FIG. 34B. The level of cyclin D1 was significantly reduced (*p<0.01) for ASO 1537S treated cells relative to control and untreated cells. FIG. 35A shows the western blot results for cyclin B1, with band quantitation shown in FIG. 35B. The level of cyclin B1 was significantly reduced (*p<0.01) for ASO 1537S or ASO-226S treated cells relative to control and untreated cells. FIG. 36 shows the western blot results for N-cadherin and caveolin. The levels of N-cadherin and caveolin were reduced for ASO 1537S treated cells relative to control and untreated cells.

### Example 16: Effects of Treatment of SK-MEL-2 and PC3 Cells with ASO-1537S or ASO-226S on Levels of Bcl-2 and FKBP38

In this experiment, SK-MEL-2 and PC3 cancer cell lines were treated with ASO-1537S or ASO-226S and levels of Bcl-2 and FKBP38 were assessed by western blot analysis. Bcl-2 is a mitochondrial anti-apoptotic protein that is kept in the mitochondria by the action of FKBP38. A reduction in the expression of FKBP38 results in the migration of Bcl-2 to the nucleus where it becomes pro-apoptotic.

### Materials and Methods

SK-MEL-2 and PC3 cells were transfected with ASO-C, ASO-226S or ASO-1537S or untreated and cultured for 24 hours as described in Example 4. Harvested cells were processed for western blot analysis as described in Example 7, using an antibody against Bcl-2 (rabbit polyclonal; Abcam; 1:1000) or FKBP38 (rabbit monoclonal; Abcam; 1:1000).

### Results

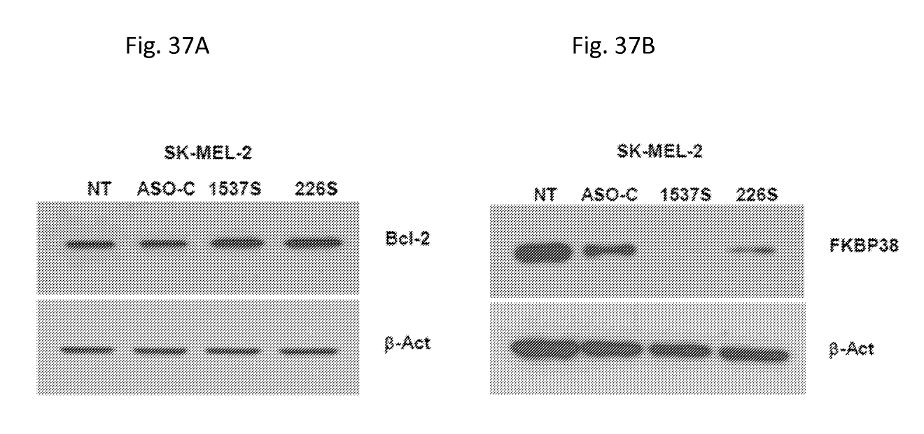

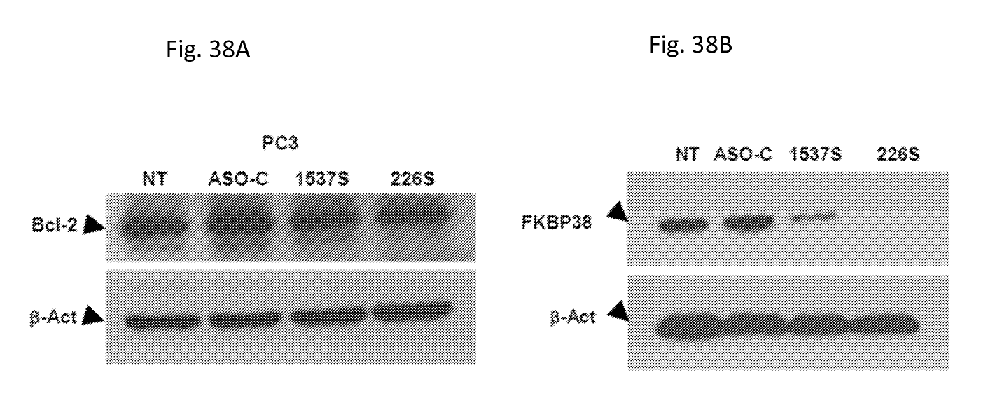

FIGS. 37A and 37B shows the western blot results for Bcl-2 and FKBP38, respectively, in SK-MEL-2 cells. The level of Bcl-2 in ASO-1537S or ASO-226S treated SK-MEL-2 cells was comparable to control and untreated cells, while the level of FKBP38 was reduced in ASO-1537S or ASO-226S treated SK-MEL-2 cells relative to control and untreated cells. FIGS. 38A and 38B shows the western blot results for Bcl-2 and FKBP38, respectively, in PC3 cells. The level of Bcl-2 in ASO-1537S or ASO-226S treated PC3 cells was comparable to control and untreated cells, while the level of FKBP38 was reduced in ASO-1537S or ASO-226S treated PC3 cells relative to control and untreated cells.

### Example 17: Down Regulation of Survivin and Cell Death in ASO-1537S Treated Cells is Dependent on RNase H

In this experiment, cell proliferation, knockdown of ASncmtRNAs (AS-1 or AS-2) and survivin levels were assessed for SK-MEL-2 cells treated with ASO-1537S or ASO-C and compared to cells treated with peptidic nucleic acid (PNA) analogs of ASO-1537S and ASO-C.

### Materials and Methods

SK-MEL-2 were transfected with 150 nM of ASO-C or ASO-1537S, PNA analogs of ASO-C and ASO-1537S or untreated. At 24 hours post transfection, cells were harvested and counted, or stained with Trypan blue (see Example 2), ASncmtRNA (AS-1, AS-2) levels were assessed by RT-PCR (see Example 1), or assessed for survivin levels by western blot analysis (see Example 12).

### Results

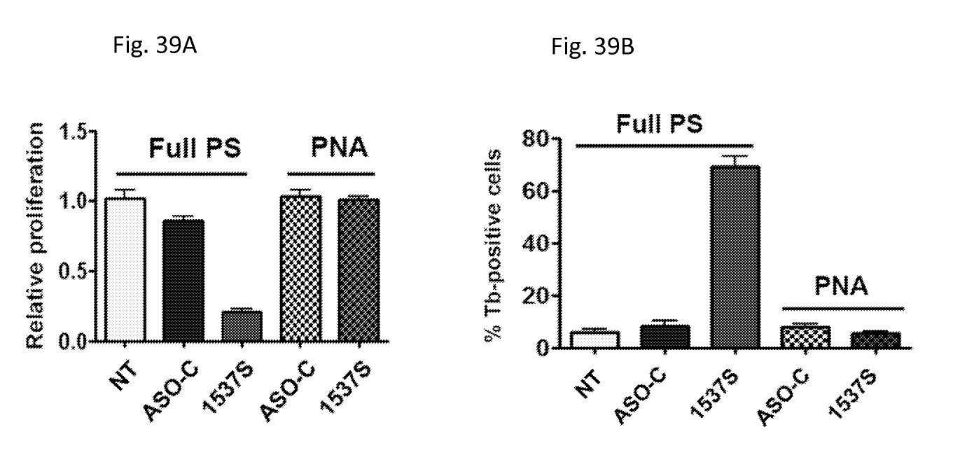

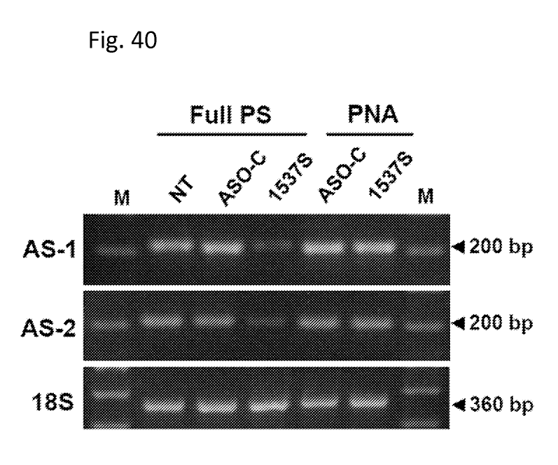

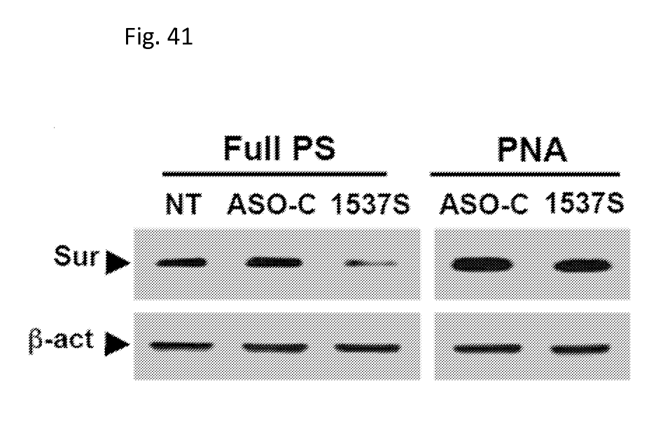

FIGS. 39A and 39B show the relative proliferation by cell counting and % Trypan blue (Tb) positive cells, respectively. The results demonstrate that ASO-1537S treated cells induced cell death, relative to control, untreated and PNA analog treated samples. Similarly, only the ASO-1537S treated cells were Tb positive relative to control, untreated and PNA analog treated samples. This demonstrates that the PNA analogs, which do not result in RNaseH degradation of the hybrid AS-1(or AS-2):ASO1537SPNA, do not result in cell death. FIG. 40 shows the knockdown of both AS-1 and AS-2 only in the ASO-1537S treated cells, with lack of knockdown in the PNA analog treated cells. FIG. 41 shows reduced levels of survivin only in the ASO-1537S treated cells. These results suggest that RNase H cleavage is necessary for the ASO-1537S to induce cell death.

### Example 18: Binding of Dicer to ASncmtRNA-1 and ASncmtRNA-2 in SK-MEL-2 Cells

In this experiment, the cytosolic fraction of SK-MEL-2 cells are immunoprecipitated with a Dicer-specific monoclonal antibody. RNA is extracted from the immunoprecipitate and RT-PCR amplification is used to assess the level of ASncmtRNA.

### Materials and Methods

SK-MEL-2 cells, 2×10⁷, were washed in 10 ml of ice cold sterile PBS, scraped, and sedimented at 300×g for 5 minutes at 4° C. The cells were lysed in 100 μl of RIP lysis buffer containing 0.5 μl of protease inhibitor and 0.25 μl of RNase inhibitor (MagnaRIP kit, Merck Millipore). An aliquot of 10 ml of each lysate was stored at −80° C. for use as input sample. A 100 μl sample of lysate was mixed with 900 μl of a suspension of magnetic beads, previously loaded with 5 μg of anti-Dicer monoclonal antibody (Abcam) or polyclonal anti-SNRNP70 or control mouse or rabbit IgG (MagnaRIP kit). The samples were incubated at room temperature for 30 minutes with rotation, followed by four hours at 4° C. with rotation. RNA/protein complexes were removed by magnetic separation and washed in 500 μl IP wash buffer, followed by 5 washes in RIP wash buffer. The recovered immunoprecipitates and input sample were incubated at 55° C. for 30 minutes in 150 μl proteinase K buffer (RIP wash buffer containing 1% SDS and 1.8 mg/ml proteinase K) under constant agitation. The magnetic beads were separated and supernatants were transferred to a different tube. A 250 μl aliquot of RIP wash buffer was added to the supernatant, followed by 400 μl phenol:chloroform:isoamyl alcohol (125:24:1, pH 4.5) with mixing. The samples were centrifuged at 21,000×g for 10 minutes at room temperature. The aqueous phase was removed and 300 μl was mixed with 50 μl of salt solution I, 15 μl of salt solution II, 5 μl of precipitate enhancer (along with salts all part of MagnaRIP kit) and 850 μl of ethanol. The RNA was allowed to precipitate overnight at 4° C. and recovered by centrifugation at 21,000×g for 30 minutes at 4° C. The pellet was washed twice in ice cold 70% ethanol, followed by entrifugation at 21,000×g for 15 minutes at 4° C. The RNA pellet was suspended in nuclease-free water and a 60 ng sample was amplified by RT-PCR as described in Example 1, with U1 snRNA control amplification using the primers supplied in the MagnaRIP kit. The primers used for amplification of AS-1 and AS-2 were as follows:

| | | |
|---|---|---|
| AS-1 | Forward | 5′-TAGGGATAACAGCGCAATCCTATT |
| | | (SEQ ID NO: 39) |
| | Reverse | 5′-ACCCACCCAAGAACAGG |
| | | (SEQ ID NO: 42) |
| AS-2 | Forward | 5′-GAACTCGGCAAACCTTACC |
| | | (SEQ ID NO: 55) |
| | Reverse | 5′-ACCCACCCAAGAACAGG |
| | | (SEQ ID NO: 42) |

### Results

FIG. 42A shows the gel results, indicating that ASncmtRNA-1 or ASncmtRNA-2 immunoprecipitated along with anti-Dicer antibody, while the control IgG sample showed no evidence of either ASncmtRNA-1 or ASncmtRNA-2. The positive control for immunoprecipitation is shown in FIG. 42B.

### Example 19: Mito-miR Generated by Dicer Cleavage of the Double Stranded Region of ASncmtRNA in Silico

In this example, the resulting double stranded region of ASncmtRNA remaining after putative cleavage of ASO/ASncmtRNA by RNase H, followed by 5′-3′ exonuclease cleavage is further cleaved by Dicer.

### Materials and Methods

The ASncmtRNA can be applied to modeling to generate approximately 22 base pair fragments as putative miRs resulting from Dicer cleavage (Park et al., Nature, 475:201-205, 2011). Using blastn alignment, TargetSCan (Lewis et al., Cell, 120:15-20, 2005) and miRbase (Griffiths-Jones et al., Nucleic Acids Res., 36:D154-158, 2008; Kozomara et al., Nucleic Acids Res., 39:D152-D157.37, 2011), putative fragments were identified.

### Results

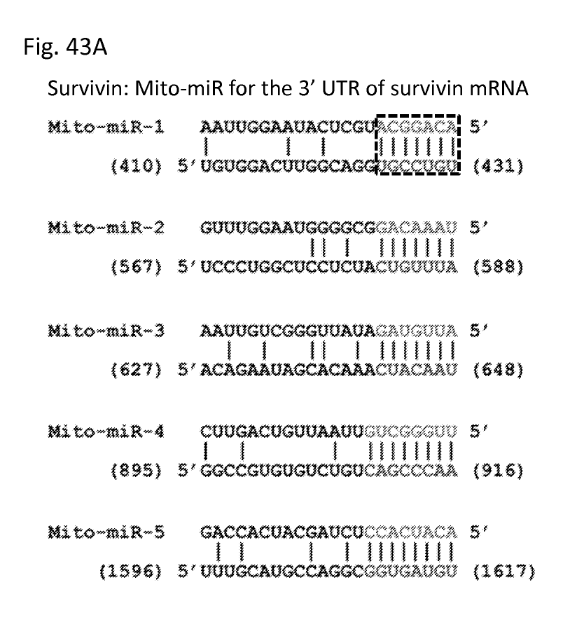

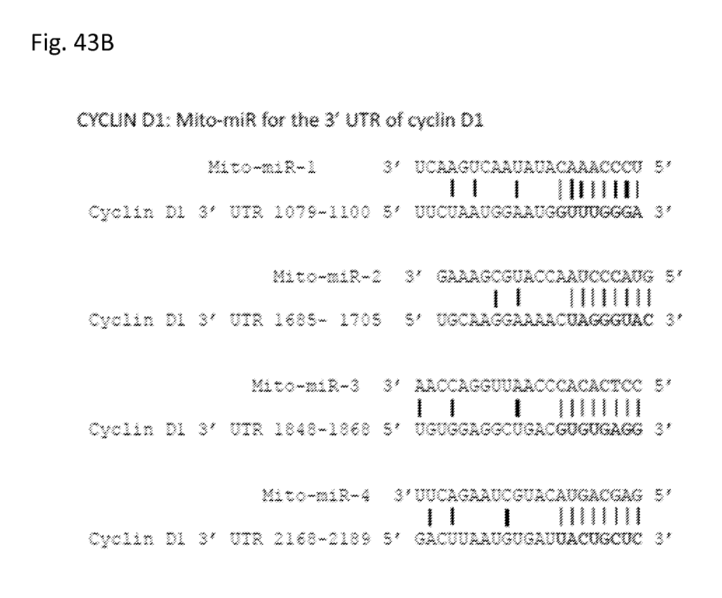

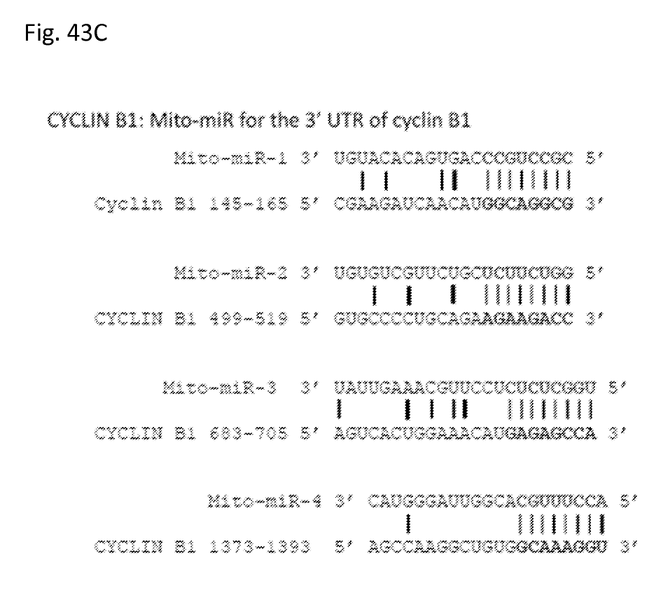

FIG. 43A-C shows the alignment of one strand of the miRs with a portion of survivin mRNA, cyclin D1 mRNA, and cyclin B1 mRNA, respectively, where the 5′ end seed region of the miR can interact with the miR recognition element located at the 3′ untranslated region (3′ UTR) of survivin mRNA, cyclin D1 mRNA and cyclin B1 mRNA (shown in blue in FIGS. 43A-43C, respectively) (Lewis, supra, Ghildiyal et al., Nat. Rev. Genet., 10:94-108,2009; Shin et al., Mol. Cell, 38:789-802, 2010; Thomas et al., Nat. Struct. Mol. Biol., 17:1169-1174, 2010; Park, supra).

### Example 20: Knock Down of ASncmtRNA Results in Inhibition of Expression of Survivin Protein

This example shows that knocking down the ASncmtRNA induces miRNAs that interact with the 3′ UTR of survivin mRNA, inhibiting the expression of the protein.

### Materials and Methods

The 3′ UTR of the survivin mRNA was amplified by RT-PCR on total SK-MEL-2 cell RNA, using forward primer 3′ UTR of survivin. The mRNA was RT-PCR-amplified from total RNA of the SK-MEL-2 cells using the following primers:

| | |
|---|---|
| Forward | 5′-AAAAAATCTAGACTT-GTTTTGTCTTG |
| | AAAGTGGCACCAG (SEQ ID NO: 37) |
| Reverse | 5′-AAAAAATCTAG-AGCACCACTTCCAGG |
| | GTTTATTCC (SEQ ID NO: 56) |

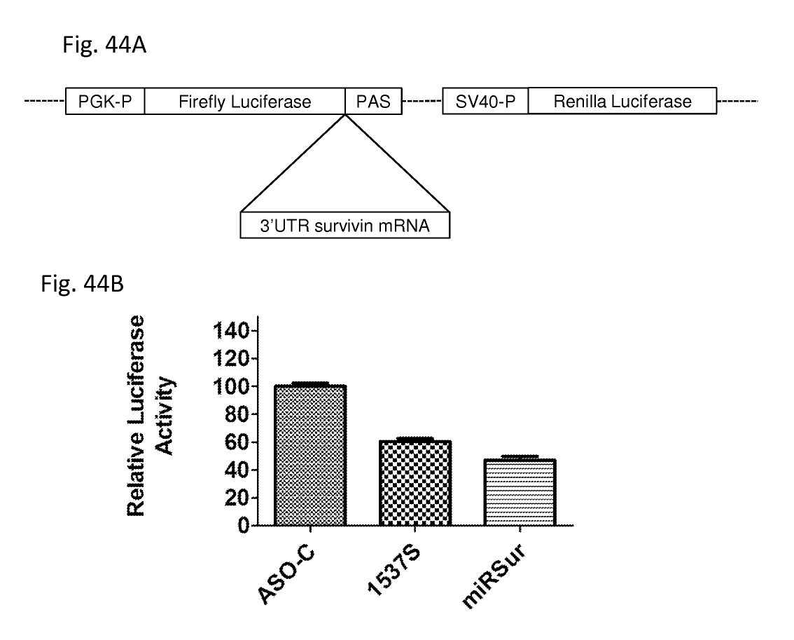

This was cloned into a unique XbaI site downstream of the firefly luciferase ORF of the pmiRGLO dual-luciferase vector (Promega) (FIG. 44A).

SK-MEL-2 cells were plated into 12-well plates (Nunc) at a density of 50,000 cells/well. The following day, cells were transfected with pmiRGLO vector containing the survivin 3′ UTR (FIG. 44A), at 0.5 μg/well, using Lipofectamine2000. At 24 hours later, cells were transfected with 150 nM ASO-C or ASO-1537S, also using Lipofectamine2000. A positive control with 20 nM survivin siRNA or mimic (Has-miR-218) was included. At 24 hours post-transfection of ASOs, cells were detached by trypsinization, sedimented at 300×g for 5 minutes at room temperature and left in a final volume of 100 μl. Seventy-five μl of each cell suspension was deposited into a well of a 96-well white plate for luminescence and firefly and Renilla luciferase activities were determined sequentially using a Synergy H4 Plate Reader and the Dual-Glo Luciferase assay system (Promega), according to manufacturer's directions. Values are expressed as the relative luciferase activity.

### Results

FIG. 44B shows that the relative activity of luciferase of SK-MEL-2 cells transfected with ASO-1537S was about 60% the ratio of the control cells transfected with ASO-C (with six independent experiments). Similarly, the ratio of cells transfected with the survivin miRNA (Has-miR-218) was about 50% of the control. Taken together, these results are a strong demonstration that knocking down the ASncmtRNA induces miRNA(s) that interact with the 3′ UTR of survivin, inhibiting the expression of the IAP family member.

The examples, which are intended to be purely exemplary of the invention and should therefore not be considered to limit the invention in any way, also describe and detail aspects and embodiments of the invention discussed above. The foregoing examples and detailed description are offered by way of illustration and not by way of limitation. All publications, patent applications, and patents cited in this specification are herein incorporated by reference as if each individual publication, patent application, or patent were specifically and individually indicated to be incorporated by reference. In particular, all publications cited herein are expressly incorporated herein by reference for the purpose of describing and disclosing compositions and methodologies which might be used in connection with the invention. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for preparing an isolated RNA molecule comprising:
(a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence;
(b) optionally digesting the non-coding chimeric mitochondrial RNA molecule cleaved by RNase H with an exonuclease to provide a non-coding chimeric mitochondrial RNA molecule sequentially cleaved by RNase H and exonuclease;
(c) optionally digesting the non-coding chimeric mitochondrial RNA molecule sequentially cleaved by RNase H and exonuclease with Dicer to provide a non-coding chimeric mitochondrial RNA molecule sequentially cleaved by RNase H, exonuclease and Dicer; and
(d) isolating the non-coding chimeric mitochondrial RNA molecule cleaved by RNase H, and/or optionally isolating the non-coding chimeric mitochondrial RNA molecule sequentially cleaved by RNase H and exonuclease, and/or optionally isolating the non-coding chimeric mitochondrial RNA molecule sequentially cleaved by RNase H, exonuclease and Dicer, to provide the isolated RNA molecule.

2. The method of claim 1, wherein the non-coding chimeric mitochondrial RNA molecule cleaved by RNase H comprises the sequence corresponding to a sequence selected from the group consisting of SEQ ID NO: 164, SEQ ID NO: 165 and SEQ ID NO: 166.

3. The method of claim 1, wherein the isolated RNA molecule is provided by isolating the non-coding chimeric mitochondrial RNA molecule cleaved by RNase H.

4. The method of claim 1, wherein the isolated RNA molecule is provided by isolating the non-coding chimeric mitochondrial RNA molecule sequentially cleaved by RNase H and exonuclease.

5. The method of claim 1, wherein the isolated RNA molecule is provided by isolating the non-coding chimeric mitochondrial RNA molecule sequentially cleaved by RNase H, exonuclease and Dicer.

6. The method of claim 5, wherein the isolated RNA molecule comprises the sequence corresponding to a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121.

7. The method of claim 5 or 6, wherein the isolated RNA molecule is a double stranded RNA molecule.

8. The method of claim 7, wherein one strand of the double stranded RNA molecule comprises the sequence corresponding to a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

9. The method of claim 7, wherein one strand of the double stranded RNA molecule consists essentially of the sequence corresponding to a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

10. The method of claim 7, wherein one strand of the double stranded RNA molecule consists of the sequence corresponding to a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

11. The method of any of claims 1-10, wherein the one or more oligonucleotides comprises the sequence corresponding to a sequence selected from the group consisting of SEQ ID NO:33, SEQ ID NO:34, SEQ ID NO:35, SEQ ID NO:36, and SEQ ID NO:45.

12. The method of any of claim 11, wherein the one or more oligonucleotides consists essentially of the sequence corresponding to a sequence selected from the group consisting of SEQ ID NO:33, SEQ ID NO:34, SEQ ID NO:35, SEQ ID NO:36, and SEQ ID NO:45.

13. The method of any of claim 11, wherein the one or more oligonucleotides consists of the sequence corresponding to a sequence selected from the group consisting of SEQ ID NO:33, SEQ ID NO:34, SEQ ID NO:35, SEQ ID NO:36, and SEQ ID NO:45.

14. An isolated RNA molecule prepared according to the method of any of claims 1-13.

15. An isolated RNA molecule comprising a sequence corresponding to a sequence corresponding to a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121.

16. A synthetic RNA molecule comprising a sequence identical to the RNA molecule prepared according to the method of any of claims 1-13.

17. A synthetic RNA molecule consisting essentially of a sequence identical to the RNA molecule prepared according to the method of any of claims 1-13.

18. A synthetic RNA molecule consisting of a sequence identical to the RNA molecule prepared according to the method of any of claims 1-13.

19. A synthetic DNA molecule comprising a sequence analogous to the RNA molecule prepared according to the method of any of claims 1-13.

20. A synthetic DNA molecule consisting essentially of a sequence analogous to the RNA molecule prepared according to the method of any of claims 1-13.

21. A synthetic DNA molecule consisting of a sequence analogous to the RNA molecule prepared according to the method of any of claims 1-13.

22. An isolated set of more than one RNA molecules, wherein said isolated set of more than one RNA molecules comprises more than one sequence identical to an RNA molecule prepared according to claim 5.

23. An isolated set of more than one RNA molecules, wherein said isolated set of more than one RNA molecules comprises sequences identical to a set of more than one RNA molecules resulting from a non-coding chimeric mitochondrial RNA molecule sequentially cleaved by RNase H, exonuclease and Dicer, wherein the non-coding chimeric mitochondrial RNA molecule sequentially cleaved by RNase H, exonuclease and Dicer is prepared by a method comprising (a) annealing one or more oligonucleotides with a non-coding chimeric mitochondrial RNA molecule and digesting with an RNase H to provide a non-coding chimeric mitochondrial RNA molecule cleaved by RNase H, wherein the one or more oligonucleotides are sufficiently complementary to the non-coding chimeric mitochondrial RNA molecule to form a stable duplex when hybridized with the non-coding chimeric mitochondrial RNA molecule, and wherein the non-coding chimeric mitochondrial RNA molecule comprises an antisense 16s mitochondrial ribosomal RNA covalently linked at its 5′ end to the 3′ end of a polynucleotide with an inverted repeat sequence; (b) digesting the non-coding chimeric mitochondrial RNA molecule cleaved by RNase H with an exonuclease to provide a non-coding chimeric mitochondrial RNA molecule sequentially cleaved by RNase H and exonuclease; and (c) digesting the non-coding chimeric mitochondrial RNA molecule sequentially cleaved by RNase H and exonuclease with Dicer to provide the set of RNA molecules resulting from the non-coding chimeric mitochondrial RNA molecule sequentially cleaved by RNase H, exonuclease and Dicer.

24. A pharmaceutical composition comprising one or more isolated or synthetic RNA molecules of any of claims 14-18 and 22-23, or one or more synthetic DNA molecules of any of claims 19-21.

25. The pharmaceutical composition of claim 24, comprising a pharmaceutically acceptable vehicle.

26. A method of causing apoptosis in a tumor cell comprising contacting the tumor cell with one or more isolated or synthetic RNA molecules of any of claims 14-18 and 22-23, or one or more synthetic DNA molecules of any of claims 19-21.

27. The method of claim 26, wherein contacting said tumor cell results in the inhibition of the expression of a protein in the tumor cell involved in apoptosis, wherein inhibition of the expression of the protein results in apoptosis of the tumor cell.

28. The method of claim 27, wherein the protein is selected from the group consisting of survivin, cyclin D1, cyclin B1, FKBP38, N-cadherin and caveolin.

29. A method of treating a cancer in a subject comprising administering to the subject in need thereof a therapeutically effective amount of one or more isolated or synthetic RNA molecules of any of claims 14-18 and 22-23, or of one or more synthetic DNA molecules of any of claims 19-21, or the pharmaceutical composition according to claim 24 or 25.

30. The method of claim 29, wherein the cancer is a hematological cancer or a solid tumor.

31. The method of claim 29, wherein administering said one or more RNA molecules or pharmaceutical composition results in the inhibition of the expression of a protein in the tumor cell involved in apoptosis, wherein inhibition of the expression of the protein results in apoptosis of the tumor cell.

32. The method of claim 31, wherein the protein is selected from the group consisting of survivin, cyclin D1, cyclin B1, FKBP38, N-cadherin and caveolin.

33. A kit for use in the treatment of cancer comprising one or more isolated or synthetic RNA molecules of any of claims 14-18 and 22-23, or one or more synthetic DNA molecules of any of claims 19-21 or the pharmaceutical composition of claim 24 or 25.

34. The kit of claim 31 comprising instructions for use of the one or more isolated or synthetic RNA molecules, or one or more synthetic DNA molecules or the pharmaceutical composition.


SEQUENCE LISTING (192 sequences)

SEQ ID NO: 1 - DNA, 2374 bp, Homo sapiens
tctaatactggtgatgctagaggtgatgtttttggtaaacaggcggggtaagatttgccgagttccttttactttttttaacctttccttatgagcatgcctgtgttgggttgacagtgagggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtgaggagttcagttatatgtttgggattttttaggtagtgggtgttgagcttgaacgctttcttaattggtggctgcttttaggcctactatgggtgttaaattttttactctctctacaaggttttttcctagtgtccaaagagctgttcctctttggactaacagttaaatttacaaggggatttagagggttctgtgggcaaatttaaagttgaactaagattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggttataatttttcatctttcccttgcggtactatatctattgcgccaggtttcaatttctatcgcctatactttatttgggtaaatggtttggctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctaccttcaaattcctccctgtacgaaaggacaagagaaataaggcctacttcacaaagcgccttcccccgtaaatgatatcatctcaacttagtattatacccacacccacccaagaacagggttt

SEQ ID NO: 2 - DNA, 1679 bp, Homo sapiens
ggggtcttagctttggctctccttgcaaagttatttctagttaattcattatgcagaaggtataggggttagtccttgctatattatgcttggttataatttttcatctttcccttgcggtgctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccaatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattatacccacacccacccaagaacagggttt

SEQ ID NO: 3 - DNA, 1635 bp, Homo sapiens
ggggtcttagctttggctctccttgcaaagttatttctagttaattcattatgcagaaggtataggggttagtccttgctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccaatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattatacccacacccacccaagaacagggttt

SEQ ID NO: 4 - DNA, 1921 bp, Homo sapiens
aacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccaatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattataccctgttcttgggtgggtgtgggtataatactaagttgagatgatatcatttacgggggaaggcgctttgtgaagtaggccttatttctcttgtcctttcgtacagggaggaatttgaagtagatagaaaccgacctggattactccggtctgaactcagatcacgtaggactttaatcgttgaacaaacgaacctttaatagcggctgcaccatcgggatgtcctgatccaacatcgaggtcgtaaaccctattgttgatatggactctagaataggattgcgctgttatccctagggtaacttgttccgttggtcaagttattggatcaattgagtatagtagttcgctttgactggtgaagtcttagcatgtactgctcggaggttgggttctgctccgaggtcgccccaaccgaaatttttaatgcaggtttggtagtttaggacctgtgggtttgttaggtactgtttgcattaataaattaaagctccatagggtcttctcgtcttgctgtgttatgcccgcctcttcacgggcaggtcaatttcactggttaaaagtaagagacagctgaaccctcgtggagccattcatacaggtccctatttaaggaacaagtgattatgctacctttgcacggttagggtaccgcggccgttaaacatgtgtcactgggcaggcggtgcctctaatactggtgatgctagaggtgatgtttttggtaaacaggcggggtaagatttgccgagttccttttactttttttaacctttccttatgagcatgcctgtgttgggttgacagtgagggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtgaggagttcagttatatgtttgggattttttaggtagtgggtgttgagcttgaacgctttcttaattggtggctgcttttaggcctactatgggtgttaaattttttactctctctacaaggttttttcctagtgtccaaagagctgttcctctttggactaacagttaaatttacaaggggatttagagggttctgtgggcaaatttaaagttgaactaagattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggttataatttttcatctttcccttgcggtactatatctattgcgccaggtttcaatttctatcgcctatactttatttgggtaaatggtttggctaaggttgtctggtagtaaggtggagtgggtttggggctaggtttagc

SEQ ID NO: 5 - DNA, 1744 bp, Homo sapiens
tagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctaccttcaaattcctccctgttcttgggtgggtgtgggtataatactaagttgagatgatatcatttacgggggaaggcgctttgtgaagtaggccttatttctcttgtcctttcgtacagggaggaatttgaagtagatagaaaccgacctggattactccggtctgaactcagatcacgtaggactttaatcgttgaacaaacgaacctttaatagcggctgcaccatcgggatgtcctgatccaacatcgaggtcgtaaaccctattgttgatatggactctagaataggattgcgctgttatccctagggtaacttgttccgttggtcaagttattggatcaattgagtatagtagttcgctttgactggtgaagtcttagcatgtactgctcggaggttgggttctgctccgaggtcgccccaaccgaaatttttaatgcaggtttggtagtttaggacctgtgggtttgttaggtactgtttgcattaataaattaaagctccatagggtcttctcgtcttgctgtgttatgcccgcctcttcacgggcaggtcaatttcactggttaaaagtaagagacagctgaaccctcgtggagccattcatacaggtccctatttaaggaacaagtgattatgctacctttgcacggttagggtaccgcggccgttaaacatgtgtcactgggcaggcggtgcctctaatactggtgatgctagaggtgatgtttttggtaaacaggcggggtaagatttgccgagttccttttactttttttaacctttccttatgagcatgcctgtgttgggttgacagtgagggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtgaggagttcagttatatgtttgggattttttaggtagtgggtgttgagcttgaacgctttcttaattggtggctgcttttaggcctactatgggtgttaaattttttactctctctacaaggttttttcctagtgtccaaagagctgttcctctttggactaacagttaaatttacaaggggatttagagggttctgtgggcaaatttaaagttgaactaagattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggttataatttttcatctttcccttgcggtactatatctattgcgccaggtttcaatttctatcgcctatactttatttgggtaaatggtttggctaaggttgtctggtagtaaggtggagtgggtttggggctaggtttagc

SEQ ID NO: 6 - DNA, 1854 bp, Homo sapiens
gaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcatgacacagcaagacgagaagaccctatggagctttaatttattaatgcaaacagtacctaacaaaccctgttcttgggtgggtgtgggtataatactaagttgagatgatatcatttacgggggaaggcgctttgtgaagtaggccttatttctcttgtcctttcgtacagggaggaatttgaagtagatagaaaccgacctggattactccggtctgaactcagatcacgtaggactttaatcgttgaacaaacgaacctttaatagcggctgcaccatcgggatgtcctgatccaacatcgaggtcgtaaaccctattgttgatatggactctagaataggattgcgctgttatccctagggtaacttgttccgttggtcaagttattggatcaattgagtatagtagttcgctttgactggtgaagtcttagcatgtactgctcggaggttgggttctgctccgaggtcgccccaaccgaaatttttaatgcaggtttggtagtttaggacctgtgggtttgttaggtactgtttgcattaataaattaaagctccatagggtcttctcgtcttgctgtgttatgcccgcctcttcacgggcaggtcaatttcactggttaaaagtaagagacagctgaaccctcgtggagccattcatacaggtccctatttaaggaacaagtgattatgctacctttgcacggttagggtaccgcggccgttaaacatgtgtcactgggcaggcggtgcctctaatactggtgatgctagaggtgatgtttttggtaaacaggcggggtaagatttgccgagttccttttactttttttaacctttccttatgagcatgcctgtgttgggttgacagtgagggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtgaggagttcagttatatgtttgggattttttaggtagtgggtgttgagcttgaacgctttcttaattggtggctgcttttaggcctactatgggtgttaaattttttactctctctacaaggttttttcctagtgtccaaagagctgttcctctttggactaacagttaaatttacaaggggatttagagggttctgtgggcaaatttaaagttgaactaagattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggttataatttttcatctttcccttgcggtactatatctattgcgccaggtttcaatttctatcgcctatactttatttgggtaaatggtttggctaaggttgtctggtagtaaggtggagtgggtttggggctaggtttagc

SEQ ID NO: 7 - RNA, 22 nt, Artificial Sequence
  Synthetic Construct
acaggcaugcucauaagguuaa

SEQ ID NO: 8 - RNA, 22 nt, Artificial Sequence
  Synthetic Construct
uaaacaggcgggguaagguuug

SEQ ID NO: 9 - RNA, 22 nt, Artificial Sequence
  Synthetic Construct
auuguagauauugggcuguuaa

SEQ ID NO: 10 - RNA, 22 nt, Artificial Sequence
  Synthetic Construct
uugggcuguuaauugucaguuc

SEQ ID NO: 11 - RNA, 22 nt, Artificial Sequence
  Synthetic Construct
acaucaccucuagcaucaccag

SEQ ID NO: 12 - RNA, 21 nt, Artificial Sequence
  Synthetic Construct
ucccaaacauauaacugaacu

SEQ ID NO: 13 - RNA, 20 nt, Artificial Sequence
  Synthetic Construct
guacccuaaccaugcgaaag

SEQ ID NO: 14 - RNA, 21 nt, Artificial Sequence
  Synthetic Construct
ccucacacccaauuggaccaa

SEQ ID NO: 15 - RNA, 21 nt, Artificial Sequence
  Synthetic Construct
gagcaguacaugcuaagacuu

SEQ ID NO: 16 - RNA, 21 nt, Artificial Sequence
  Synthetic Construct
cgccugcccagugacacaugu

SEQ ID NO: 17 - RNA, 21 nt, Artificial Sequence
  Synthetic Construct
ggucuucucgucuugcugugu

SEQ ID NO: 18 - RNA, 23 nt, Artificial Sequence
  Synthetic Construct
uggcucucuccuugcaaaguuau

SEQ ID NO: 19 - RNA, 21 nt, Artificial Sequence
  Synthetic Construct
accuuugcacgguuaggguac

SEQ ID NO: 20 - RNA, 20 nt, Artificial Sequence
  Synthetic Construct
aaccuuaugagcaugccugu

SEQ ID NO: 21 - RNA, 20 nt, Artificial Sequence
  Synthetic Construct
aaccuuaccccgccuguuua

SEQ ID NO: 22 - RNA, 20 nt, Artificial Sequence
  Synthetic Construct
aacagcccaauaucuacaau

SEQ ID NO: 23 - RNA, 20 nt, Artificial Sequence
  Synthetic Construct
acugacaauuaacagcccaa

SEQ ID NO: 24 - RNA, 20 nt, Artificial Sequence
  Synthetic Construct
ggugaugcuagaggugaugu

SEQ ID NO: 25 - RNA, 19 nt, Artificial Sequence
  Synthetic Construct
uucaguuauauguuuggga

SEQ ID NO: 26 - RNA, 18 nt, Artificial Sequence
  Synthetic Construct
uucgcaugguuaggguac

SEQ ID NO: 27 - RNA, 19 nt, Artificial Sequence
  Synthetic Construct
gguccaauugggugugagg

SEQ ID NO: 28 - RNA, 19 nt, Artificial Sequence
  Synthetic Construct
gucuuagcauguacugcuc

SEQ ID NO: 29 - RNA, 19 nt, Artificial Sequence
  Synthetic Construct
augugucacugggcaggcg

SEQ ID NO: 30 - RNA, 19 nt, Artificial Sequence
  Synthetic Construct
acagcaagacgagaagacc

SEQ ID NO: 31 - RNA, 21 nt, Artificial Sequence
  Synthetic Construct
aacuuugcaaggagagagcca

SEQ ID NO: 32 - RNA, 19 nt, Artificial Sequence
  Synthetic Construct
acccuaaccgugcaaaggu

SEQ ID NO: 33 - DNA, 20 bp, Artificial Sequence
  Synthetic Construct
agatgaaaaattataaccaa

SEQ ID NO: 34 - DNA, 20 bp, Artificial Sequence
  Synthetic Construct
tacctaaaaaatcccaaaca

SEQ ID NO: 35 - DNA, 18 bp, Artificial Sequence
  Synthetic Construct
gtcctaaactaccaaacc

SEQ ID NO: 36 - DNA, 18 bp, Artificial Sequence
  Synthetic Construct
cacccacccaagaacagg

SEQ ID NO: 37 - DNA, 39 bp, Artificial Sequence
  Synthetic Construct
aaaaaatctagacttgttttgtcttgaaagtggcaccag

SEQ ID NO: 38 - DNA, 18 bp, Artificial Sequence
  Synthetic Construct
aggtggagtggattgggg

SEQ ID NO: 39 - DNA, 24 bp, Artificial Sequence
  Synthetic Construct
tagggataacagcgcaatcctatt

SEQ ID NO: 40 - DNA, 25 bp, Artificial Sequence
  Synthetic Construct
cacacccacccaagaacagggagga

SEQ ID NO: 41 - DNA, 23 bp, Artificial Sequence
  Synthetic Construct
accgtgcaaaggtagcataatca

SEQ ID NO: 42 - DNA, 17 bp, Artificial Sequence
  Synthetic Construct
acccacccaagaacagg

SEQ ID NO: 43 - DNA, 19 bp, Artificial Sequence
  Synthetic Construct
agtggactcattccaatta

SEQ ID NO: 44 - DNA, 15 bp, Artificial Sequence
  Synthetic Construct
gatgcgtgcatttat

SEQ ID NO: 45 - DNA, 20 bp, Artificial Sequence
  Synthetic Construct
taagacccccgaaaccagac

SEQ ID NO: 46 - DNA, 16 bp, Artificial Sequence
  Synthetic Construct
atgggtgccccgacgt

SEQ ID NO: 47 - DNA, 23 bp, Artificial Sequence
  Synthetic Construct
aatgtagagatgcggtggtcctt

SEQ ID NO: 48 - DNA, 21 bp, Artificial Sequence
  Synthetic Construct
cccctgcctggcagccctttc

SEQ ID NO: 49 - DNA, 17 bp, Artificial Sequence
  Synthetic Construct
aatcacctaatgcccac

SEQ ID NO: 50 - DNA, 17 bp, Artificial Sequence
  Synthetic Construct
tgttcttgcctgtcttg

SEQ ID NO: 51 - DNA, 22 bp, Artificial Sequence
  Synthetic Construct
cagagatcctgctcttaaacgc

SEQ ID NO: 52 - DNA, 20 bp, Artificial Sequence
  Synthetic Construct
gtaacccgttgaaccccatt

SEQ ID NO: 53 - DNA, 19 bp, Artificial Sequence
  Synthetic Construct
catccaatcggtagtagcg

SEQ ID NO: 54 - DNA, 26 bp, Artificial Sequence
  Synthetic Construct
agtaagtgcgggtcataagcttgcgt

SEQ ID NO: 55 - DNA, 19 bp, Artificial Sequence
  Synthetic Construct
gaactcggcaaaccttacc

SEQ ID NO: 56 - DNA, 35 bp, Artificial Sequence
  Synthetic Construct
aaaaaatctagagcaccacttccagggtttattcc

SEQ ID NO: 57 - DNA, 22 bp, Artificial Sequence
  Synthetic Construct
acaggcatgctcataaggttaa

SEQ ID NO: 58 - DNA, 22 bp, Artificial Sequence
  Synthetic Construct
taaacaggcggggtaaggtttg

SEQ ID NO: 59 - DNA, 22 bp, Artificial Sequence
  Synthetic Construct
attgtagatattgggctgttaa

SEQ ID NO: 60 - DNA, 22 bp, Artificial Sequence
  Synthetic Construct
ttgggctgttaattgtcagttc

SEQ ID NO: 61 - DNA, 22 bp, Artificial Sequence
  Synthetic Construct
acatcacctctagcatcaccag

SEQ ID NO: 62 - DNA, 21 bp, Artificial Sequence
  Synthetic Construct
tcccaaacatataactgaact

SEQ ID NO: 63 - DNA, 20 bp, Artificial Sequence
  Synthetic Construct
gtaccctaaccatgcgaaag

SEQ ID NO: 64 - DNA, 21 bp, Artificial Sequence
  Synthetic Construct
cctcacacccaattggaccaa

SEQ ID NO: 65 - DNA, 21 bp, Artificial Sequence
  Synthetic Construct
gagcagtacatgctaagactt

SEQ ID NO: 66 - DNA, 21 bp, Artificial Sequence
  Synthetic Construct
cgcctgcccagtgacacatgt

SEQ ID NO: 67 - DNA, 21 bp, Artificial Sequence
  Synthetic Construct
ggtcttctcgtcttgctgtgt

SEQ ID NO: 68 - DNA, 23 bp, Artificial Sequence
  Synthetic Construct
tggctctctccttgcaaagttat

SEQ ID NO: 69 - DNA, 21 bp, Artificial Sequence
  Synthetic Construct
acctttgcacggttagggtac

SEQ ID NO: 70 - DNA, 20 bp, Artificial Sequence
  Synthetic Construct
aaccttatgagcatgcctgt

SEQ ID NO: 71 - DNA, 20 bp, Artificial Sequence
  Synthetic Construct
aaccttaccccgcctgttta

SEQ ID NO: 72 - DNA, 20 bp, Artificial Sequence
  Synthetic Construct
aacagcccaatatctacaat

SEQ ID NO: 73 - DNA, 20 bp, Artificial Sequence
  Synthetic Construct
actgacaattaacagcccaa

SEQ ID NO: 74 - DNA, 20 bp, Artificial Sequence
  Synthetic Construct
ggtgatgctagaggtgatgt

SEQ ID NO: 75 - DNA, 19 bp, Artificial Sequence
  Synthetic Construct
ttcagttatatgtttggga

SEQ ID NO: 76 - DNA, 18 bp, Artificial Sequence
  Synthetic Construct
ttcgcatggttagggtac

SEQ ID NO: 77 - DNA, 19 bp, Artificial Sequence
  Synthetic Construct
ggtccaattgggtgtgagg

SEQ ID NO: 78 - DNA, 19 bp, Artificial Sequence
  Synthetic Construct
gtcttagcatgtactgctc

SEQ ID NO: 79 - DNA, 19 bp, Artificial Sequence
  Synthetic Construct
atgtgtcactgggcaggcg

SEQ ID NO: 80 - DNA, 19 bp, Artificial Sequence
  Synthetic Construct
acagcaagacgagaagacc

SEQ ID NO: 81 - DNA, 21 bp, Artificial Sequence
  Synthetic Construct
aactttgcaaggagagagcca

SEQ ID NO: 82 - DNA, 19 bp, Artificial Sequence
  Synthetic Construct
accctaaccgtgcaaaggt

SEQ ID NO: 83 - RNA, 22 nt, Artificial Sequence
  Synthetic construct
aaccuuaugagcaugccugunn

SEQ ID NO: 84 - RNA, 22 nt, Artificial Sequence
  Synthetic construct
aaccuuaccccgccuguuuann

SEQ ID NO: 85 - RNA, 22 nt, Artificial Sequence
  Synthetic construct
aacagcccaauaucuacaaunn

SEQ ID NO: 86 - RNA, 22 nt, Artificial Sequence
  Synthetic construct
acugacaauuaacagcccaann

SEQ ID NO: 87 - RNA, 22 nt, Artificial Sequence
  Synthetic construct
ggugaugcuagaggugaugunn

SEQ ID NO: 88 - RNA, 21 nt, Artificial Sequence
  Synthetic construct
uucaguuauauguuugggann

SEQ ID NO: 89 - RNA, 20 nt, Artificial Sequence
  Synthetic construct
uucgcaugguuaggguacnn

SEQ ID NO: 90 - RNA, 21 nt, Artificial Sequence
  Synthetic construct
gguccaauugggugugaggnn

SEQ ID NO: 91 - RNA, 21 nt, Artificial Sequence
  Synthetic construct
gucuuagcauguacugcucnn

SEQ ID NO: 92 - RNA, 21 nt, Artificial Sequence
  Synthetic construct
augugucacugggcaggcgnn

SEQ ID NO: 93 - RNA, 21 nt, Artificial Sequence
  Synthetic construct
acagcaagacgagaagaccnn

SEQ ID NO: 94 - RNA, 23 nt, Artificial Sequence
  Synthetic construct
aacuuugcaaggagagagccann

SEQ ID NO: 95 - RNA, 21 nt, Artificial Sequence
  Synthetic construct
acccuaaccgugcaaaggunn

SEQ ID NO: 96 - RNA, 19 nt, Artificial Sequence
  Synthetic construct
accuuaugagcaugccugu

SEQ ID NO: 97 - RNA, 19 nt, Artificial Sequence
  Synthetic construct
accuuaccccgccuguuua

SEQ ID NO: 98 - RNA, 19 nt, Artificial Sequence
  Synthetic construct
acagcccaauaucuacaau

SEQ ID NO: 99 - RNA, 19 nt, Artificial Sequence
  Synthetic construct
cugacaauuaacagcccaa

SEQ ID NO: 100 - RNA, 19 nt, Artificial Sequence
  Synthetic construct
gugaugcuagaggugaugu

SEQ ID NO: 101 - RNA, 18 nt, Artificial Sequence
  Synthetic construct
ucaguuauauguuuggga

SEQ ID NO: 102 - RNA, 17 nt, Artificial Sequence
  Synthetic construct
ucgcaugguuaggguac

SEQ ID NO: 103 - RNA, 18 nt, Artificial Sequence
  Synthetic construct
guccaauugggugugagg

SEQ ID NO: 104 - RNA, 18 nt, Artificial Sequence
  Synthetic construct
ucuuagcauguacugcuc

SEQ ID NO: 105 - RNA, 18 nt, Artificial Sequence
  Synthetic construct
ugugucacugggcaggcg

SEQ ID NO: 106 - RNA, 18 nt, Artificial Sequence
  Synthetic construct
cagcaagacgagaagacc

SEQ ID NO: 107 - RNA, 20 nt, Artificial Sequence
  Synthetic construct
acuuugcaaggagagagcca

SEQ ID NO: 108 - RNA, 18 nt, Artificial Sequence
  Synthetic construct
cccuaaccgugcaaaggu

SEQ ID NO: 109 - RNA, 22 nt, Artificial Sequence
  Synthetic construct
accuuaugagcaugccugunnn

SEQ ID NO: 110 - RNA, 22 nt, Artificial Sequence
  Synthetic construct
accuuaccccgccuguuuannn

SEQ ID NO: 111 - RNA, 22 nt, Artificial Sequence
  Synthetic construct
acagcccaauaucuacaaunnn

SEQ ID NO: 112 - RNA, 22 nt, Artificial Sequence
  Synthetic construct
cugacaauuaacagcccaannn

SEQ ID NO: 113 - RNA, 22 nt, Artificial Sequence
  Synthetic construct
gugaugcuagaggugaugunnn

SEQ ID NO: 114 - RNA, 21 nt, Artificial Sequence
  Synthetic construct
ucaguuauauguuugggannn

SEQ ID NO: 115 - RNA, 20 nt, Artificial Sequence
  Synthetic construct
ucgcaugguuaggguacnnn

SEQ ID NO: 116 - RNA, 21 nt, Artificial Sequence
  Synthetic construct
guccaauugggugugaggnnn

SEQ ID NO: 117 - RNA, 21 nt, Artificial Sequence
  Synthetic construct
ucuuagcauguacugcucnnn

SEQ ID NO: 118 - RNA, 21 nt, Artificial Sequence
  Synthetic construct
ugugucacugggcaggcgnnn

SEQ ID NO: 119 - RNA, 21 nt, Artificial Sequence
  Synthetic construct
cagcaagacgagaagaccnnn

SEQ ID NO: 120 - RNA, 23 nt, Artificial Sequence
  Synthetic construct
acuuugcaaggagagagccannn

SEQ ID NO: 121 - RNA, 21 nt, Artificial Sequence
  Synthetic construct
cccuaaccgugcaaaggunnn

SEQ ID NO: 122 - DNA, 22 bp, Artificial Sequence
  Synthetic construct
aaccttatgagcatgcctgtnn

SEQ ID NO: 123 - DNA, 22 bp, Artificial Sequence
  Synthetic construct
aaccttaccccgcctgtttann

SEQ ID NO: 124 - DNA, 22 bp, Artificial Sequence
  Synthetic construct
aacagcccaatatctacaatnn

SEQ ID NO: 125 - DNA, 22 bp, Artificial Sequence
  Synthetic construct
actgacaattaacagcccaann

SEQ ID NO: 126 - DNA, 22 bp, Artificial Sequence
  Synthetic construct
ggtgatgctagaggtgatgtnn

SEQ ID NO: 127 - DNA, 21 bp, Artificial Sequence
  Synthetic construct
ttcagttatatgtttgggann

SEQ ID NO: 128 - DNA, 20 bp, Artificial Sequence
  Synthetic construct
ttcgcatggttagggtacnn

SEQ ID NO: 129 - DNA, 21 bp, Artificial Sequence
  Synthetic construct
ggtccaattgggtgtgaggnn

SEQ ID NO: 130 - DNA, 21 bp, Artificial Sequence
  Synthetic construct
gtcttagcatgtactgctcnn

SEQ ID NO: 131 - DNA, 21 bp, Artificial Sequence
  Synthetic construct
atgtgtcactgggcaggcgnn

SEQ ID NO: 132 - DNA, 21 bp, Artificial Sequence
  Synthetic construct
acagcaagacgagaagaccnn

SEQ ID NO: 133 - DNA, 23 bp, Artificial Sequence
  Synthetic construct
aactttgcaaggagagagccann

SEQ ID NO: 134 - DNA, 21 bp, Artificial Sequence
  Synthetic construct
accctaaccgtgcaaaggtnn

SEQ ID NO: 135 - DNA, 19 bp, Artificial Sequence
  Synthetic construct
accttatgagcatgcctgt

SEQ ID NO: 136 - DNA, 19 bp, Artificial Sequence
  Synthetic construct
accttaccccgcctgttta

SEQ ID NO: 137 - DNA, 19 bp, Artificial Sequence
  Synthetic construct
acagcccaatatctacaat

SEQ ID NO: 138 - DNA, 19 bp, Artificial Sequence
  Synthetic construct
ctgacaattaacagcccaa

SEQ ID NO: 139 - DNA, 19 bp, Artificial Sequence
  Synthetic construct
gtgatgctagaggtgatgt

SEQ ID NO: 140 - DNA, 18 bp, Artificial Sequence
  Synthetic construct
tcagttatatgtttggga

SEQ ID NO: 141 - DNA, 17 bp, Artificial Sequence
  Synthetic construct
tcgcatggttagggtac

SEQ ID NO: 142 - DNA, 18 bp, Artificial Sequence
  Synthetic construct
gtccaattgggtgtgagg

SEQ ID NO: 143 - DNA, 18 bp, Artificial Sequence
  Synthetic construct
tcttagcatgtactgctc

SEQ ID NO: 144 - DNA, 18 bp, Artificial Sequence
  Synthetic construct
tgtgtcactgggcaggcg

SEQ ID NO: 145 - DNA, 18 bp, Artificial Sequence
  Synthetic construct
cagcaagacgagaagacc

SEQ ID NO: 146 - DNA, 20 bp, Artificial Sequence
  Synthetic construct
actttgcaaggagagagcca

SEQ ID NO: 147 - DNA, 18 bp, Artificial Sequence
  Synthetic construct
ccctaaccgtgcaaaggt

SEQ ID NO: 148 - DNA, 22 bp, Artificial Sequence
  Synthetic construct
accttatgagcatgcctgtnnn

SEQ ID NO: 149 - DNA, 22 bp, Artificial Sequence
  Synthetic construct
accttaccccgcctgtttannn

SEQ ID NO: 150 - DNA, 22 bp, Artificial Sequence
  Synthetic construct
acagcccaatatctacaatnnn

SEQ ID NO: 151 - DNA, 22 bp, Artificial Sequence
  Synthetic construct
ctgacaattaacagcccaannn

SEQ ID NO: 152 - DNA, 22 bp, Artificial Sequence
  Synthetic construct
gtgatgctagaggtgatgtnnn

SEQ ID NO: 153 - DNA, 21 bp, Artificial Sequence
  Synthetic construct
tcagttatatgtttgggannn

SEQ ID NO: 154 - DNA, 20 bp, Artificial Sequence
  Synthetic construct
tcgcatggttagggtacnnn

SEQ ID NO: 155 - DNA, 21 bp, Artificial Sequence
  Synthetic construct
gtccaattgggtgtgaggnnn

SEQ ID NO: 156 - DNA, 21 bp, Artificial Sequence
  Synthetic construct
tcttagcatgtactgctcnnn

SEQ ID NO: 157 - DNA, 21 bp, Artificial Sequence
  Synthetic construct
tgtgtcactgggcaggcgnnn

SEQ ID NO: 158 - DNA, 21 bp, Artificial Sequence
  Synthetic construct
cagcaagacgagaagaccnnn

SEQ ID NO: 159 - DNA, 23 bp, Artificial Sequence
  Synthetic construct
actttgcaaggagagagccannn

SEQ ID NO: 160 - DNA, 21 bp, Artificial Sequence
  Synthetic construct
ccctaaccgtgcaaaggtnnn

SEQ ID NO: 161 - RNA, 2374 nt, Homo sapiens
ucuaauacuggugaugcuagaggugauguuuuugguaaacaggcgggguaagauuugccgaguuccuuuuacuuuuuuuaaccuuuccuuaugagcaugccuguguuggguugacagugaggguaauaaugacuuguugguugauuguagauauugggcuguuaauugucaguucaguguuuuaaucugacgcaggcuuaugcggaggagaauguuuucauguuacuuauacuaacauuaguucuucuauagggugauagauugguccaauugggugugaggaguucaguuauauguuugggauuuuuuagguaguggguguugagcuugaacgcuuucuuaauugguggcugcuuuuaggccuacuauggguguuaaauuuuuuacucucucuacaagguuuuuuccuaguguccaaagagcuguuccucuuuggacuaacaguuaaauuuacaaggggauuuagaggguucugugggcaaauuuaaaguugaacuaagauucuaucuuggacaaccagcuaucaccaggcucgguagguuugucgccucuaccuauaaaucuucccacuauuuugcuacauagacgggugugcucuuuuagcuguucuuagguagcucgucugguuucgggggucuuagcuuuggcucuccuugcaaaguuauuucuaguuaauucauuaugcagaagguauagggguuaguccuugcuauauuaugcuugguuauaauuuuucaucuuucccuugcgguacuauaucuauugcgccagguuucaauuucuaucgccuauacuuuauuuggguaaaugguuuggcuaaaccuagccccaaacccacuccaccuuacuaccagacaaccuuagccaaaccauuuacccaaauaaaguauaggcgauagaaauugaaaccuggcgcaauagauauaguaccgcaagggaaagaugaaaaauuauaaccaagcauaauauagcaaggacuaaccccuauaccuucugcauaaugaauuaacuagaaauaacuuugcaaggagagccaaagcuaagacccccgaaaccagacgagcuaccuaagaacagcuaaaagagcacacccgucuauguagcaaaauagugggaagauuuauagguagaggcgacaaaccuaccgagccuggugauagcugguuguccaagauagaaucuuaguucaacuuuaaauuugcccacagaacccucuaaauccccuuguaaauuuaacuguuaguccaaagaggaacagcucuuuggacacuaggaaaaaaccuuguagagagaguaaaaaauuuaacacccauaguaggccuaaaagcagccaccaauuaagaaagcguucaagcucaacacccacuaccuaaaaaaucccaaacauauaacugaacuccucacacccaauuggaccaaucuaucacccuauagaagaacuaauguuaguauaaguaacaugaaaacauucuccuccgcauaagccugcgucagauuaaaacacugaacugacaauuaacagcccaauaucuacaaucaaccaacaagucauuauuacccucacugucaacccaacacaggcaugcucauaaggaaagguuaaaaaaaguaaaaggaacucggcaaaucuuaccccgccuguuuaccaaaaacaucaccucuagcaucaccaguauuagaggcaccgccugcccagugacacauguuuaacggccgcgguacccuaaccgugcaaagguagcauaaucacuuguuccuuaaauagggaccuguaugaauggcuccacgaggguucagcugucucuuacuuuuaaccagugaaauugaccugcccgugaagaggcgggcauaacacagcaagacgagaagacccuauggagcuuuaauuuauuaaugcaaacaguaccuaacaaacccacagguccuaaacuaccaaaccugcauuaaaaauuucgguuggggcgaccucggagcagaacccaaccuccgagcaguacaugcuaagacuucaccagucaaagcgaacuacuauacucaauugauccaauaacuugaccaacggaacaaguuacccuagggauaacagcgcaauccuauucuagaguccauaucaacaauaggguuuacgaccucgauguuggaucaggacaucccgauggugcagccgcuauuaaagguucguuuguucaacgauuaaaguccuacgugaucugaguucagaccggaguaauccaggucgguuucuaucuaccuucaaauuccucccuguacgaaaggacaagagaaauaaggccuacuucacaaagcgccuucccccguaaaugauaucaucucaacuuaguauuauacccacacccacccaagaacaggguuu

SEQ ID NO: 162 - RNA, 1679 nt, Homo sapiens
ggggucuuagcuuuggcucuccuugcaaaguuauuucuaguuaauucauuaugcagaagguauagggguuaguccuugcuauauuaugcuugguuauaauuuuucaucuuucccuugcggugcuaaaccuagccccaaacccacuccaccuuacuaccagacaaccuuagccaaaccauuuacccaaauaaaguauaggcgauagaaauugaaaccuggcgcaauagauauaguaccgcaagggaaagaugaaaaauuauaaccaagcauaauauagcaaggacuaaccccuauaccuucugcauaaugaauuaacuagaaauaacuuugcaaggagagccaaagcuaagacccccgaaaccagacgagcuaccuaagaacagcuaaaagagcacacccgucuauguagcaaaauagugggaagauuuauagguagaggcgacaaaccuaccgagccuggugauagcugguuguccaagauagaaucuuaguucaacuuuaaauuugcccacagaacccucuaaauccccuuguaaauuuaacuguuaguccaaagaggaacagcucuuuggacacuaggaaaaaaccuuguagagagaguaaaaaauuuaacacccauaguaggccuaaaagcagccaccaauuaagaaagcguucaagcucaacacccacuaccuaaaaaaucccaaacauauaacugaacuccucacacccaauuggaccaaucuaucacccuauagaagaacuaauguuaguauaaguaacaugaaaacauucuccuccgcauaagccugcgucagauuaaaacacugaacugacaauuaacagcccaauaucuacaaucaaccaacaagucauuauuacccucacugucaacccaacacaggcaugcucauaaggaaagguuaaaaaaaguaaaaggaacucggcaaaucuuaccccgccuguuuaccaaaaacaucaccucuagcaucaccaguauuagaggcaccgccugcccagugacacauguuuaacggccgcgguacccuaaccgugcaaagguagcauaaucacuuguuccuuaaauagggaccuguaugaauggcuccacgaggguucagcugucucuuacuuuuaaccagugaaauugaccugcccgugaagaggcgggcauaacacagcaagacgagaagacccuauggagcuuuaauuuauuaaugcaaacaguaccuaacaaacccacagguccuaaacuaccaaaccugcauuaaaaauuucgguuggggcgaccucggagcagaacccaaccuccgagcaguacaugcuaagacuucaccagucaaagcgaacuacuauacucaauugauccaauaacuugaccaacggaacaaguuacccuagggauaacagcgcaauccuauucuagaguccauaucaacaauaggguuuacgaccucgauguuggaucaggacaucccaauggugcagccgcuauuaaagguucguuuguucaacgauuaaaguccuacgugaucugaguucagaccggaguaauccaggucgguuucuaucuacuucaaauuccucccuguacgaaaggacaagagaaauaaggccuacuucacaaagcgccuucccccguaaaugauaucaucucaacuuaguauuauacccacacccacccaagaacaggguuu

SEQ ID NO: 163 - RNA, 1635 nt, Homo sapiens
ggggucuuagcuuuggcucuccuugcaaaguuauuucuaguuaauucauuaugcagaagguauagggguuaguccuugcuaaaccuagccccaaacccacuccaccuuacuaccagacaaccuuagccaaaccauuuacccaaauaaaguauaggcgauagaaauugaaaccuggcgcaauagauauaguaccgcaagggaaagaugaaaaauuauaaccaagcauaauauagcaaggacuaaccccuauaccuucugcauaaugaauuaacuagaaauaacuuugcaaggagagccaaagcuaagacccccgaaaccagacgagcuaccuaagaacagcuaaaagagcacacccgucuauguagcaaaauagugggaagauuuauagguagaggcgacaaaccuaccgagccuggugauagcugguuguccaagauagaaucuuaguucaacuuuaaauuugcccacagaacccucuaaauccccuuguaaauuuaacuguuaguccaaagaggaacagcucuuuggacacuaggaaaaaaccuuguagagagaguaaaaaauuuaacacccauaguaggccuaaaagcagccaccaauuaagaaagcguucaagcucaacacccacuaccuaaaaaaucccaaacauauaacugaacuccucacacccaauuggaccaaucuaucacccuauagaagaacuaauguuaguauaaguaacaugaaaacauucuccuccgcauaagccugcgucagauuaaaacacugaacugacaauuaacagcccaauaucuacaaucaaccaacaagucauuauuacccucacugucaacccaacacaggcaugcucauaaggaaagguuaaaaaaaguaaaaggaacucggcaaaucuuaccccgccuguuuaccaaaaacaucaccucuagcaucaccaguauuagaggcaccgccugcccagugacacauguuuaacggccgcgguacccuaaccgugcaaagguagcauaaucacuuguuccuuaaauagggaccuguaugaauggcuccacgaggguucagcugucucuuacuuuuaaccagugaaauugaccugcccgugaagaggcgggcauaacacagcaagacgagaagacccuauggagcuuuaauuuauuaaugcaaacaguaccuaacaaacccacagguccuaaacuaccaaaccugcauuaaaaauuucgguuggggcgaccucggagcagaacccaaccuccgagcaguacaugcuaagacuucaccagucaaagcgaacuacuauacucaauugauccaauaacuugaccaacggaacaaguuacccuagggauaacagcgcaauccuauucuagaguccauaucaacaauaggguuuacgaccucgauguuggaucaggacaucccaauggugcagccgcuauuaaagguucguuuguucaacgauuaaaguccuacgugaucugaguucagaccggaguaauccaggucgguuucuaucuacuucaaauuccucccuguacgaaaggacaagagaaauaaggccuacuucacaaagcgccuucccccguaaaugauaucaucucaacuuaguauuauacccacacccacccaagaacaggguuu

SEQ ID NO: 164 - RNA, 1921 nt, Homo sapiens
aaccuccgagcaguacaugcuaagacuucaccagucaaagcgaacuacuauacucaauugauccaauaacuugaccaacggaacaaguuacccuagggauaacagcgcaauccuauucuagaguccauaucaacaauaggguuuacgaccucgauguuggaucaggacaucccaauggugcagccgcuauuaaagguucguuuguucaacgauuaaaguccuacgugaucugaguucagaccggaguaauccaggucgguuucuaucuacuucaaauuccucccuguacgaaaggacaagagaaauaaggccuacuucacaaagcgccuucccccguaaaugauaucaucucaacuuaguauuauacccuguucuuggguggguguggguauaauacuaaguugagaugauaucauuuacgggggaaggcgcuuugugaaguaggccuuauuucucuuguccuuucguacagggaggaauuugaaguagauagaaaccgaccuggauuacuccggucugaacucagaucacguaggacuuuaaucguugaacaaacgaaccuuuaauagcggcugcaccaucgggauguccugauccaacaucgaggucguaaacccuauuguugauauggacucuagaauaggauugcgcuguuaucccuaggguaacuuguuccguuggucaaguuauuggaucaauugaguauaguaguucgcuuugacuggugaagucuuagcauguacugcucggagguuggguucugcuccgaggucgccccaaccgaaauuuuuaaugcagguuugguaguuuaggaccuguggguuuguuagguacuguuugcauuaauaaauuaaagcuccauagggucuucucgucuugcuguguuaugcccgccucuucacgggcaggucaauuucacugguuaaaaguaagagacagcugaacccucguggagccauucauacaggucccuauuuaaggaacaagugauuaugcuaccuuugcacgguuaggguaccgcggccguuaaacaugugucacugggcaggcggugccucuaauacuggugaugcuagaggugauguuuuugguaaacaggcgggguaagauuugccgaguuccuuuuacuuuuuuuaaccuuuccuuaugagcaugccuguguuggguugacagugaggguaauaaugacuuguugguugauuguagauauugggcuguuaauugucaguucaguguuuuaaucugacgcaggcuuaugcggaggagaauguuuucauguuacuuauacuaacauuaguucuucuauagggugauagauugguccaauugggugugaggaguucaguuauauguuugggauuuuuuagguaguggguguugagcuugaacgcuuucuuaauugguggcugcuuuuaggccuacuauggguguuaaauuuuuuacucucucuacaagguuuuuuccuaguguccaaagagcuguuccucuuuggacuaacaguuaaauuuacaaggggauuuagaggguucugugggcaaauuuaaaguugaacuaagauucuaucuuggacaaccagcuaucaccaggcucgguagguuugucgccucuaccuauaaaucuucccacuauuuugcuacauagacgggugugcucuuuuagcuguucuuagguagcucgucugguuucgggggucuuagcuuuggcucuccuugcaaaguuauuucuaguuaauucauuaugcagaagguauagggguuaguccuugcuauauuaugcuugguuauaauuuuucaucuuucccuugcgguacuauaucuauugcgccagguuucaauuucuaucgccuauacuuuauuuggguaaaugguuuggcuaagguugucugguaguaagguggaguggguuuggggcuagguuuagc

SEQ ID NO: 165 - RNA, 1744 nt, Homo sapiens
uagggauaacagcgcaauccuauucuagaguccauaucaacaauaggguuuacgaccucgauguuggaucaggacaucccgauggugcagccgcuauuaaagguucguuuguucaacgauuaaaguccuacgugaucugaguucagaccggaguaauccaggucgguuucuaucuaccuucaaauuccucccuguucuuggguggguguggguauaauacuaaguugagaugauaucauuuacgggggaaggcgcuuugugaaguaggccuuauuucucuuguccuuucguacagggaggaauuugaaguagauagaaaccgaccuggauuacuccggucugaacucagaucacguaggacuuuaaucguugaacaaacgaaccuuuaauagcggcugcaccaucgggauguccugauccaacaucgaggucguaaacccuauuguugauauggacucuagaauaggauugcgcuguuaucccuaggguaacuuguuccguuggucaaguuauuggaucaauugaguauaguaguucgcuuugacuggugaagucuuagcauguacugcucggagguuggguucugcuccgaggucgccccaaccgaaauuuuuaaugcagguuugguaguuuaggaccuguggguuuguuagguacuguuugcauuaauaaauuaaagcuccauagggucuucucgucuugcuguguuaugcccgccucuucacgggcaggucaauuucacugguuaaaaguaagagacagcugaacccucguggagccauucauacaggucccuauuuaaggaacaagugauuaugcuaccuuugcacgguuaggguaccgcggccguuaaacaugugucacugggcaggcggugccucuaauacuggugaugcuagaggugauguuuuugguaaacaggcgggguaagauuugccgaguuccuuuuacuuuuuuuaaccuuuccuuaugagcaugccuguguuggguugacagugaggguaauaaugacuuguugguugauuguagauauugggcuguuaauugucaguucaguguuuuaaucugacgcaggcuuaugcggaggagaauguuuucauguuacuuauacuaacauuaguucuucuauagggugauagauugguccaauugggugugaggaguucaguuauauguuugggauuuuuuagguaguggguguugagcuugaacgcuuucuuaauugguggcugcuuuuaggccuacuauggguguuaaauuuuuuacucucucuacaagguuuuuuccuaguguccaaagagcuguuccucuuuggacuaacaguuaaauuuacaaggggauuuagaggguucugugggcaaauuuaaaguugaacuaagauucuaucuuggacaaccagcuaucaccaggcucgguagguuugucgccucuaccuauaaaucuucccacuauuuugcuacauagacgggugugcucuuuuagcuguucuuagguagcucgucugguuucgggggucuuagcuuuggcucuccuugcaaaguuauuucuaguuaauucauuaugcagaagguauagggguuaguccuugcuauauuaugcuugguuauaauuuuucaucuuucccuugcgguacuauaucuauugcgccagguuucaauuucuaucgccuauacuuuauuuggguaaaugguuuggcuaagguugucugguaguaagguggaguggguuuggggcuagguuuagc

SEQ ID NO: 166 - RNA, 1854 nt, Homo sapiens
gaacucggcaaaucuuaccccgccuguuuaccaaaaacaucaccucuagcaucaccaguauuagaggcaccgccugcccagugacacauguuuaacggccgcgguacccuaaccgugcaaagguagcauaaucacuuguuccuuaaauagggaccuguaugaauggcuccacgaggguucagcugucucuuacuuuuaaccagugaaauugaccugcccgugaagaggcgggcaugacacagcaagacgagaagacccuauggagcuuuaauuuauuaaugcaaacaguaccuaacaaacccuguucuuggguggguguggguauaauacuaaguugagaugauaucauuuacgggggaaggcgcuuugugaaguaggccuuauuucucuuguccuuucguacagggaggaauuugaaguagauagaaaccgaccuggauuacuccggucugaacucagaucacguaggacuuuaaucguugaacaaacgaaccuuuaauagcggcugcaccaucgggauguccugauccaacaucgaggucguaaacccuauuguugauauggacucuagaauaggauugcgcuguuaucccuaggguaacuuguuccguuggucaaguuauuggaucaauugaguauaguaguucgcuuugacuggugaagucuuagcauguacugcucggagguuggguucugcuccgaggucgccccaaccgaaauuuuuaaugcagguuugguaguuuaggaccuguggguuuguuagguacuguuugcauuaauaaauuaaagcuccauagggucuucucgucuugcuguguuaugcccgccucuucacgggcaggucaauuucacugguuaaaaguaagagacagcugaacccucguggagccauucauacaggucccuauuuaaggaacaagugauuaugcuaccuuugcacgguuaggguaccgcggccguuaaacaugugucacugggcaggcggugccucuaauacuggugaugcuagaggugauguuuuugguaaacaggcgggguaagauuugccgaguuccuuuuacuuuuuuuaaccuuuccuuaugagcaugccuguguuggguugacagugaggguaauaaugacuuguugguugauuguagauauugggcuguuaauugucaguucaguguuuuaaucugacgcaggcuuaugcggaggagaauguuuucauguuacuuauacuaacauuaguucuucuauagggugauagauugguccaauugggugugaggaguucaguuauauguuugggauuuuuuagguaguggguguugagcuugaacgcuuucuuaauugguggcugcuuuuaggccuacuauggguguuaaauuuuuuacucucucuacaagguuuuuuccuaguguccaaagagcuguuccucuuuggacuaacaguuaaauuuacaaggggauuuagaggguucugugggcaaauuuaaaguugaacuaagauucuaucuuggacaaccagcuaucaccaggcucgguagguuugucgccucuaccuauaaaucuucccacuauuuugcuacauagacgggugugcucuuuuagcuguucuuagguagcucgucugguuucgggggucuuagcuuuggcucuccuugcaaaguuauuucuaguuaauucauuaugcagaagguauagggguuaguccuugcuauauuaugcuugguuauaauuuuucaucuuucccuugcgguacuauaucuauugcgccagguuucaauuucuaucgccuauacuuuauuuggguaaaugguuuggcuaagguugucugguaguaagguggaguggguuuggggcuagguuuagc

SEQ ID NO: 167 - RNA, 22 nt, Artificial Sequence
  Synthetic Construct
acaggcaugcucauaagguuaa

SEQ ID NO: 168 - RNA, 22 nt, Homo sapiens
uguggacuuggcaggugccugu

SEQ ID NO: 169 - RNA, 22 nt, Artificial Sequence
  Synthetic Construct
uaaacaggcgggguaagguuug

SEQ ID NO: 170 - RNA, 22 nt, Homo sapiens
ucccuggcuccucuacuguuua

SEQ ID NO: 171 - RNA, 22 nt, Artificial Sequence
  Synthetic Construct
auuguagauauugggcuguuaa

SEQ ID NO: 172 - RNA, 22 nt, Homo sapiens
acagaauagcacaaacuacaau

SEQ ID NO: 173 - RNA, 22 nt, Artificial Sequence
  Synthetic Construct
uugggcuguuaauugucaguuc

SEQ ID NO: 174 - RNA, 22 nt, Homo sapiens
ggccgugugucugucagcccaa

SEQ ID NO: 175 - RNA, 22 nt, Artificial Sequence
  Synthetic Construct
acaucaccucuagcaucaccag

SEQ ID NO: 176 - RNA, 22 nt, Homo sapiens
uuugcaugccaggcggugaugu

SEQ ID NO: 177 - RNA, 21 nt, Artificial Sequence
  Synthetic Construct
ucccaaacauauaacugaacu

SEQ ID NO: 178 - RNA, 21 nt, Homo sapiens
uucuaauggaaugguuuggga

SEQ ID NO: 179 - RNA, 20 nt, Artificial Sequence
  Synthetic Construct
guacccuaaccaugcgaaag

SEQ ID NO: 180 - RNA, 20 nt, Homo sapiens
ugcaaggaaaacuaggguac

SEQ ID NO: 181 - RNA, 21 nt, Artificial Sequence
  Synthetic Construct
ccncacacccaauuggaccaa

SEQ ID NO: 182 - RNA, 21 nt, Homo sapiens
uguggaggcugacgugugagg

SEQ ID NO: 183 - RNA, 21 nt, Artificial Sequence
  Synthetic Construct
gagcaguacaugcuaagacuu

SEQ ID NO: 184 - RNA, 21 nt, Homo sapiens
gacuuaaugugauuacugcuc

SEQ ID NO: 185 - RNA, 21 nt, Artificial Sequence
  Synthetic Construct
cgccugcccagugacacaugu

SEQ ID NO: 186 - RNA, 21 nt, Homo sapiens
cgaagaucaacauggcaggcg

SEQ ID NO: 187 - RNA, 21 nt, Artificial Sequence
  Synthetic Construct
ggucuucucgucuugcugugu

SEQ ID NO: 188 - RNA, 21 nt, Homo sapiens
gugccccugcagaagaagacc

SEQ ID NO: 189 - RNA, 23 nt, Artificial Sequence
  Synthetic Construct
uggcucucuccuugcaaaguuau

SEQ ID NO: 190 - RNA, 23 nt, Homo sapiens
agucacuggaaacaugagagcca

SEQ ID NO: 191 - RNA, 21 nt, Artificial Sequence
  Synthetic Construct
accuuugcacgguuaggguac

SEQ ID NO: 192 - RNA, 21 nt, Homo sapiens
agccaaggcuguggcaaaggu